(19) United States
(12) Patent Application Publication
(10) Pub. No.: US 2011/0263557 A1
(43) Pub. Date: Oct. 27, 2011

(54) COMPOUNDS AND COMPOSITIONS AS MODULATORS OF GPR119 ACTIVITY

(75) Inventors: Christopher Cow, Poway, CA (US); Robert Epple, San Diego, CA (US)

(73) Assignee: IRM LLC, Hamilton (BM)

(21) Appl. No.: 12/936,453

(22) PCT Filed: Apr. 3, 2009
(86) PCT No.: PCT/US09/39506

§ 371 Date: Feb. 25, 2011

Related U.S. Application Data
(60) Provisional application No. 61/043,100, filed on Apr. 7, 2008.

(51) Int. Cl. A61K 31/506; C12N 5/07; C07D 401/12; A61K 31/496; C07D 413/14; A61K 31/5377; A61P 3/04; A61P 3/10; A61P 3/06; A61P 9/00; A61P 9/10; A61P 19/02; A61P 19/10; A61P 9/12; A61P 9/04; A61P 13/12; A61P 3/00; A61P 1/04; A61P 15/10; C07D 401/14

(52) U.S. Cl. 514/210.2; 544/332; 514/252.18; 435/375; 544/365; 514/253.12; 544/121; 514/235.8; 514/275

(57) ABSTRACT

The invention provides compounds, pharmaceutical compositions comprising such compounds and methods of using such compounds to treat or prevent diseases or disorders associated with the activity of GPR119; such as, but not limited to, diabetes, obesity and associated metabolic disorders. Formula (I) is a compound, in which A can have up to 2 ring —CH2- group substituted with —C(O)— and can be partially unsaturated with up to 2 double bonds; Wi and W2 are independently selected from CR10 and N; wherein R10 is selected from hydrogen and C1_6alkyl; Yi is selected from NRn, O and S; wherein Rn is selected from hydrogen and C1_6alkyl; Y2 and Y3 are independently selected from CH and N; Y4 is selected from CH2, OCH2 and NR15; wherein R15 is selected from hydrogen and C1_6alkyl; or the pharmaceutically acceptable salts thereof.

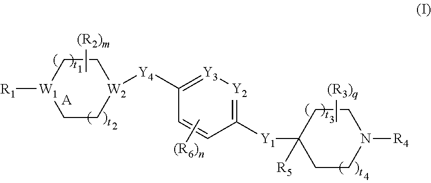

### CROSS-REFERENCE TO RELATED APPLICATION

This application claims the benefit of priority to U.S. Provisional Patent Application No. 61/043,100, filed 7 Apr. 2008. The full disclosure of this application is incorporated herein by reference in its entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activity of GPR119.

2. Background

GPR119 is a G-protein coupled receptor (GPCR) that is mainly expressed in the pancreas, small intestine, colon and adipose tissue. The expression profile of the human GPR119 receptor indicates its potential utility as a target for the treatment of obesity and diabetes. The novel compounds of this invention modulate the activity of GPR119 and are, therefore, expected to be useful in the treatment of GPR119-associated diseases or disorders such as, but not limited to, diabetes, obesity and associated metabolic disorders.

### SUMMARY OF THE INVENTION

In one aspect, the present invention provides a compound of Formula I:

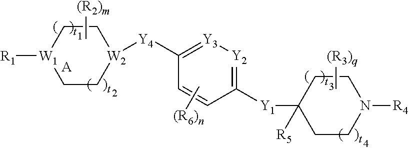

in which:

A can have up to 2 ring —CH₂— group substituted with —C(O)— and can be partially unsaturated with up to 2 double bonds;

m and n are independently selected from 0, 1, 2, 3 and 4;

q is selected from 0, 1, 2, 3 and 4;

t₁, t₂, t₃ and t₄ are each independently selected from 0, 1 and 2;

R₁ is selected from hydrogen, cyano, —X₁S(O)₀₋₂X₂R₆ₐ, —X₁N(S(O)₀₋₂X₂R₆ₐ)R₆ₐ, —X₁S(O)₀₋₂X₂OR₆ₐ, —X₁S(O)₀₋₂X₂C(O)R₆ₐ, —X₁C(O)OR₆ₐ, —X₁R₆ₐ, —X₁S(O)₀₋₂X₂C(O)OR₆ₐ and —X₁S(O)₀₋₂NR₆ₐR_{6b}; wherein X₁ is selected from a bond, O, —NR₇ₐR_{7b} and C₁₋₄alkylene; X₂ is selected from a bond and C₁₋₄alkylene; R₆ₐ is selected from hydrogen, C₁₋₆alkyl, C₆₋₁₀aryl, C₁₋₁₀heteroaryl, C₃₋₈heterocycloalkyl and C₁₋₈cycloalkyl; wherein said aryl, heteroaryl, cycloalkyl and heterocycloalkyl of R₆ₐ is optionally substituted with 1 to 3 radicals independently selected from hydroxy, halo, C₁₋₆alkyl, halo-substituted-C₁₋₆alkyl, hydroxy-substituted-C₁₋₆alkyl, C₁₋₆alkoxy, halo-substituted-C₁₋₆alkoxy and C₆₋₁₀aryl-C₁₋₄alkoxy; R_{6b} is selected from hydrogen and C₁₋₆alkyl; and R₇ₐ and R_{7b} are independently selected from hydrogen and C₁₋₆alkyl;

R₂ and R₃ are independently selected from halo, hydroxy, C₁₋₆alkyl, halo-substituted-C₁₋₆alkyl, hydroxy-substituted-C₁₋₆alkyl, C₁₋₆alkoxy, halo-substituted-C₁₋₆alkoxy, —C(O)R₈, and —C(O)OR₈; wherein R₈ is selected from hydrogen and C₁₋₆alkyl;

R₄ is selected from R₉ and —C(O)OR₉; wherein R₉ is selected from C₁₋₆alkyl, C₆₋₁₀aryl, C₁₋₁₀heteroaryl, C₃₋₈cycloalkyl and C₃₋₈heterocycloalkyl; wherein said aryl, heteroaryl, cycloalkyl or heterocycloalkyl of R₉ is optionally substituted with 1 to 3 radicals independently selected from halo, cyano, C₁₋₆alkyl, C₃₋₁₂cycloalkyl, C₃₋₈heterocycloalkyl, halo-substituted-C₁₋₆alkyl, hydroxy-substituted-C₁₋₆alkyl, C₁₋₆alkoxy, halo-substituted-C₁₋₆alkoxy and —C(O)OR₁₇, —C(O)R₁₉ and —C(O)NR₁₇R₁₈; wherein R₁₇ and R₁₈ are independently selected from hydrogen and C₁₋₆alkyl; or R₁₇ and R₁₈ together with the nitrogen atom to which R₁₇ and R₁₈ are attached form C₃₋₈heterocycloalkyl; R₁₉ is selected from C₁₋₆alkyl and C₃₋₈heterocycloalkyl; wherein said cycloalkyl or heterocycloalkyl substituents of R₉ are optionally further substituted with 1 to 3 C₁₋₆alkyl radicals;

R₅ is selected from hydrogen, C₁₋₆alkyl, halo-substituted-C₁₋₆alkyl, hydroxy-substituted-C₁₋₆alkyl, C₁₋₆alkoxy and halo-substituted-C₁₋₆alkoxy;

R₆ is selected from hydroxy, nitro, cyano, halo, C₁₋₆alkyl, C₂₋₆alkenyl, halo-substituted-C₁₋₆alkyl, halo-substituted-C₂₋₆alkenyl, hydroxy-substituted-C₁₋₆alkyl, C₁₋₆alkoxy, halo-substituted-C₁₋₆alkoxy, C₆₋₁₀aryl, C₁₋₁₀heteroaryl, C₃₋₈heterocycloalkyl, C₃₋₈cycloalkyl and —X₃OR₂₀, —NR₂₀X₃OR₂₁, —C(O)OR₂₀; wherein X₃ is selected from a bond, C₁₋₄alkylene and C₂₋₄alkenylene; R₂₀ and R₂₁ are independently selected from hydrogen and C₁₋₆alkyl;

W₁ and W₂ are independently selected from CR₁₀ and N; wherein R₁₀ is selected from hydrogen and C₁₋₆alkyl;

Y₁ is selected from NR₁₁, O and S; wherein R₁₁ is selected from hydrogen and C₁₋₆alkyl;

Y₂ and Y₃ are independently selected from CH and N; and

Y₄ is selected from CH₂, OCH₂ and NR₁₅; wherein R₁₅ is selected from hydrogen and C₁₋₆alkyl.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of GPR119 activity can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which GPR119 activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be straight-chained, branched, cyclic or spiro. C₁₋₆alkoxy includes methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group. “Heteroaryl” is as defined for aryl where one or more of the ring members are a heteroatom. For example, C₁₋₁₀heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, 1H-pyridin-2-onyl, 6-oxo-1,6-dihydro-pyridin-3-yl, etc. “C₆₋₁₀arylC₀₋₄alkyl” means an aryl as described above connected via a alkylene grouping. For example, C₆₋₁₀arylC₀₋₄alkyl includes phenethyl, benzyl, etc. Heteroaryl also includes the N-oxide derivatives, for example, pyridine N-oxide derivatives with the following structure:

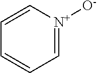

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, 2-oxo-pyrrolidin-1-yl, 2-oxo-piperidin-1-yl, etc.

GPR119 means G protein-coupled receptor 119 (GenBank® Accession No. AAP72125) is also referred to in the literature as RUP3 and GPR116. The term GPR119 as used herein includes the human sequences found in GeneBank accession number AY288416, naturally-occurring allelic variants, mammalian orthologs, and recombinant mutants thereof.

“Halogen” (or halo) preferably represents chloro or fluoro, but can also be bromo or iodo.

“Treat”, “treating” and “treatment” refer to a method of alleviating or abating a disease and/or its attendant symptoms.

### Description of the Preferred Embodiments

The present invention provides compounds, compositions and methods for the treatment of diseases in which modulation of GPR119 activity can prevent, inhibit or ameliorate the pathology and/or symptomology of the diseases, which method comprises administering to the animal a therapeutically effective amount of a compound of Formula I.

In one embodiment, with reference to compounds of Formula I, are compounds of Formula Ia:

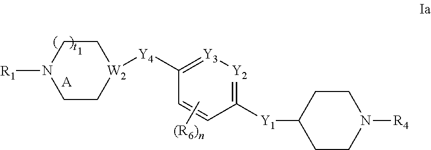

in which:

A can have a ring —CH₂— group substituted with —C(O)—;

t1 is selected from 0 and 1;

R₁ is selected from hydrogen, cyano, —X₁S(O)₀₋₂X₂R₆ₐ, —X₁S(O)₀₋₂X₂R₆ₐ, —X₁C(O)OR₆ₐ, —X₁S(O)₀₋₂X₂C(O)R₆ₐ, —X₁N(S(O)₀₋₂X₂R₆ₐ)R₆ₐ, —X₁R₆ₐ, —X₁S(O)₀₋₂X₂C(O)OR₆ₐ and —X₁S(O)₀₋₂NR₆ₐR_{6b}; wherein X₁ is selected from a bond, O, —NR₇ₐR_{7b} and C₁₋₄alkylene; X₂ is selected from a bond and C₁₋₄alkylene; R₆ₐ is selected from hydrogen, C₁₋₆ alkyl, C₆₋₁₀aryl, C₁₋₁₀heteroaryl, C₃₋₈heterocycloalkyl and C₁₋₈cycloalkyl; wherein said aryl, heteroaryl, cycloalkyl and heterocycloalkyl of R₆ₐ is optionally substituted with 1 to 3 radicals independently selected from hydroxy, halo, C₁₋₆alkyl, halo-substituted-C₁₋₆alkyl, hydroxy-substituted-C₁₋₆alkyl, C₁₋₆alkoxy, halo-substituted-C₁₋₆alkoxy and C₆₋₁₀aryl-C₁₋₄alkoxy; R_{6b} is selected from hydrogen and C₁₋₆alkyl; and R₇ₐ and R_{7b} are independently selected from hydrogen and C₁₋₆alkyl;

R₄ is selected from R₉ and —C(O)OR₉; wherein R₉ is selected from C₁₋₆alkyl, C₆₋₁₀aryl, C₁₋₁₀heteroaryl, C₃₋₈ cycloalkyl and C₃₋₈heterocycloalkyl; wherein said aryl, heteroaryl, cycloalkyl or heterocycloalkyl of R₉ is optionally substituted with 1 to 3 radicals independently selected from halo, cyano, C₁₋₆alkyl, C₃₋₁₂cycloalkyl, C₃₋₈heterocycloalkyl, halo-substituted-C₁₋₆ alkyl, hydroxy-substituted-C₁₋₆alkyl, C₁₋₆alkoxy, halo-substituted-C₁₋₆alkoxy and —C(O)OR₁₇, —C(O)R₁₉ and —C(O)NR₁₇R₁₈; wherein R₁₇ and R₁₈ are independently selected from hydrogen and C₁₋₆alkyl; or R₁₇ and R₁₈ together with the nitrogen atom to which R₁₇ and R₁₈ are attached form C₃₋₈heterocycloalkyl; R₁₉ is selected from C₁₋₆alkyl and C₃₋₈heterocycloalkyl; wherein said cycloalkyl or heterocycloalkyl substituents of R₉ are optionally further substituted with 1 to 3 C₁₋₆alkyl radicals;

R₆ is selected from hydroxy, nitro, cyano, halo, C₁₋₆alkyl, C₂₋₆alkenyl, halo-substituted-C₁₋₆alkyl, halo-substituted-C₂₋₆alkenyl, hydroxy-substituted-C₁₋₆alkyl, C₁₋₆alkoxy, halo-substituted-C₁₋₆alkoxy, C₆₋₁₀aryl, C₁₋₁₀heteroaryl, C₃₋₈heterocycloalkyl, C₃₋₈cycloalkyl and —X₃OR₂₀, —NR₂₀X₃OR₂₁, —C(O)OR₂₀; wherein X₃ is selected from a bond, C₁₋₄alkylene and C₂₋₄alkenylene; R₂₀ and R₂₁ are independently selected from hydrogen and C₁₋₆alkyl;

W₂ is selected from CR₁₀ and N; wherein R₁₀ is selected from hydrogen and C₁₋₆alkyl;

Y₁ is selected from NH, O and S; and

Y₂ and Y₃ are independently selected from CH and N;

Y₄ is selected from CH₂, OCH₂ and NR₁₅; wherein R₁₅ is selected from hydrogen and C₁₋₆alkyl.

In another embodiment, A can have a ring —CH₂— group substituted with —C(O)—; t1 is selected from 0 and 1; and R₁ is selected from hydrogen, cyano, —S(O)₀₋₂X₂R₆ₐ, —X₁N(S(O)₀₋₂X₂R₆ₐ)R₆ₐ, —X₁R₆ₐ, —X₁C(O)OR₆ₐ and —S(O)₀₋₂X₂OR₆ₐ; wherein X₁ is selected from a bond and C₁₋₄alkylene; X₂ is selected from a bond and C₁₋₄alkylene; R₆ₐ is selected from hydrogen, C₁₋₆alkyl and C₁₋₁₀heteroaryl optionally substituted with C₁₋₆alkyl.

In another embodiment, R₄ is selected from R₉ and —C(O)OR₉; wherein R₉ is selected from tert-butyl, pyridinyl, pyrimidinyl, 1,2,4-oxadiazol-5-yl, tetrazolyl and cyclopropyl; wherein said pyridinyl, pyrimidinyl, 1,2,4-oxadiazol-5-yl, tetrazolyl or cyclopropyl of R₉ is optionally substituted with a radical selected from halo, cyano, trifluoromethyl, isopropyl, methyl, ethyl, methoxy-carbonyl, dimethyl-amino-carbonyl, amino-carbonyl and morpholino-carbonyl.

In another embodiment, R₆ is selected from fluoro, chloro, bromo, trifluoromethoxy, methyl, methoxy, methoxy-carbonyl, 3-methoxyprop-1-enyl, methoxy-propyl, vinyl, phenyl, pyrazolyl, 5-chloropent-1-enyl, hydroxy-propyl, methoxy-ethyl-amino and morpholino; W₂ is selected from CH and N; Y₁ is selected from NH, O and S; and Y₂ and Y₃ are independently selected from CH and N; Y₄ is selected from CH₂, OCH₂ and NCH₃.

In another embodiment are compounds selected from: 5-Ethyl-2-(4-(4-((4-(methylsulfonyl)piperazin-1-yl)methyl)phenoxy)piperidin-1-yl)pyrimidine; 5-ethyl-2-(4-(3-methyl-4-((4-(methylsulfonyl)piperazin-1-yl)methyl)phenoxy)piperidin-1-yl)pyrimidine; 5-ethyl-2-(4-(3-methoxy-4-((4-(methylsulfonyl)piperazin-1-yl)methyl)phenoxy)piperidin-1-yl)pyrimidine; 5-ethyl-2-(4-(3-fluoro-4-((4-(methylsulfonyl)piperazin-1-yl)methyl)phenoxy)piperidin-1-yl)pyrimidine; 2-(4-(3-chloro-4-((4-(methylsulfonyl)piperazin-1-yl)methyl)phenoxy)piperidin-1-yl)-5-ethylpyrimidine; 5-ethyl-2-(4-(2-methyl-4-((4-(methylsulfonyl)piperazin-1-yl)methyl)phenoxy)piperidin-1-yl)pyrimidine; 5-ethyl-2-(4-(2-fluoro-4-((4-(methylsulfonyl)piperazin-1-yl)methyl)phenoxy)piperidin-1-yl)pyrimidine; 2-(4-(2-chloro-4-((4-(methylsulfonyl)piperazin-1-yl)methyl)phenoxy)piperidin-1-yl)-5-ethylpyrimidine; 2-(4-(2-bromo-4-((4-(methylsulfonyl)piperazin-1-yl)methyl)phenoxy)piperidin-1-yl)-5-ethylpyrimidine; 5-ethyl-2-(4-(2-methoxy-4-((4-(methylsulfonyl)piperazin-1-yl)methyl)phenoxy)piperidin-1-yl)pyrimidine; 5-ethyl-2-(4-(4-((4-(methylsulfonyl)piperazin-1-yl)methyl)-2-(trifluoromethoxy)phenoxy)piperidin-1-yl)pyrimidine; 2-(4-(2,3-difluoro-4-((4-(methylsulfonyl)piperazin-1-yl)methyl)phenoxy)piperidin-1-yl)-5-ethylpyrimidine; 2-(4-(2-chloro-6-fluoro-4-((4-(methylsulfonyl)piperazin-1-yl)methyl)phenoxy)piperidin-1-yl)-5-ethylpyrimidine; 5-ethyl-2-(4-(6-((4-(methylsulfonyl)piperazin-1-yl)methyl)pyridin-3-yloxy)piperidin-1-yl)pyrimidine; 5-ethyl-2-(4-(5-((4-(methylsulfonyl)piperazin-1-yl)methyl)pyridin-2-yloxy)piperidin-1-yl)pyrimidine; tert-Butyl 4-(4-((4-(methylsulfonyl)piperazin-1-yl)methyl)phenoxy)piperidine-1-carboxylate; 1-methylcyclopropyl 4-(4-((4-(methylsulfonyl)piperazin-1-yl)methyl)phenoxy)piperidine-1-carboxylate; 5-Fluoro-2-(4-(4-((4-(methylsulfonyl)piperazin-1-yl)methyl)phenoxy)piperidin-1-yl)pyrimidine; 1-(4-(1-(5-fluoropyridin-2-yl)piperidin-4-yloxy)benzyl)-4-(methylsulfonyl)piperazine; 1-(methylsulfonyl)-4-(4-(1-(5-(trifluoromethyl)pyridin-2-yl)piperidin-4-yloxy)benzyl)piperazine; 5-(4-(2-chloro-4-((4-(methylsulfonyl)piperazin-1-yl)methyl)phenoxy)piperidin-1-yl)-3-isopropyl-1,2,4-oxadiazole; 3-(4-(3-Chloro-4-(1-(5-ethylpyrimidin-2-yl)piperidin-4-yloxy)benzyl)piperazin-1-ylsulfonyl)propan-1-ol; 5-Ethyl-2-(4-(4-((1-(methylsulfonyl)piperidin-4-yl)methyl)phenoxy)piperidin-1-yl)pyrimidine; tert-Butyl 4-(2-fluoro-4-((4-(methylsulfonyl)piperazin-1-yl)methyl)phenylamino) piperidine-1-carboxylate; 1-(3-Chloro-4-(1-(5-ethylpyrimidin-2-yl)piperidin-4-yloxy)benzyl)-4-(methylsulfonyl)piperazin-2-one, methyl 2-(1-(5-ethylpyrimidin-2-yl)piperidin-4-yloxy)-5-((4-(methylsulfonyl)piperazin-1-yl)methyl)benzoate; 1-methylcyclopropyl 4-(2-chloro-4-((4-(methylsulfonyl)piperazin-1-yl)methyl)phenoxy)piperidine-1-carboxylate; methyl 2-(4-(2-chloro-4-((4-(methylsulfonyl)piperazin-1-yl)methyl)phenoxy)piperidin-1-yl)pyrimidine-5-carboxylate; 2-(4-(2-chloro-4-((4-(methylsulfonyl)piperazin-1-yl)methyl)phenoxy)piperidin-1-yl)pyrimidine; 5-bromo-2-(4-(2-chloro-4-((4-(methylsulfonyl)piperazin-1-yl)methyl)-phenoxy)piperidin-1-yl)pyrimidine; 5-chloro-2-(4-(2-chloro-4-((4-(methylsulfonyl)piperazin-1-yl)methyl)phenoxy)piperidin-1-yl)pyrimidine; 2-(4-(2-Chloro-4-((4-(methylsulfonyl)-piperazin-1-yl)methyl)phenoxy)piperidin-1-yl)pyrimidine-5-carboxamide; 2-(4-(2-chloro-4-((4-(methylsulfonyl)piperazin-1-yl)methyl)phenoxy)piperidin-1-yl)-N,N-dimethylpyrimidine-5-carboxamide; (2-(4-(2-chloro-4-((4-(methylsulfonyl)piperazin-1-yl)methyl)phenoxy)piperidin-1-yl)pyrimidin-5-yl)(morpholino)methanone; 2-(4-(2-Chloro-4-((4-(methylsulfonyl)piperazin-1-yl)methyl)phenoxy)piperidin-1-yl)pyrimidine-5-carbonitrile; 2-(4-(2-Chloro-4-((4-(methylsulfonyl)piperazin-1-yl)methyl)phenoxy)piperidin-1-yl)-5-(2H-tetrazol-5-yl)pyrimidine; 2-(4-(2-Chloro-4-((4-(methylsulfonyl)piperazin-1-yl)methyl)phenoxy)piperidin-1-yl)-5-(2-methyl-2H-tetrazol-5-yl)pyrimidine; (E)-5-Ethyl-2-(4-(2-(3-methoxyprop-1-enyl)-4-((4-(methylsulfonyl)piperazin-1-yl)methyl)phenoxy)piperidin-1-yl)pyrimidine; 3-(2-(1-(5-Ethylpyrimidin-2-yl)piperidin-4-yloxy)-5-((4-(methylsulfonyl)piperazin-1-yl)methyl)phenyl)propan-1-ol; 5-ethyl-2-(4-(4-((4-(methylsulfonyl)piperazin-1-yl)methyl)-2-vinylphenoxy)piperidin-1-yl)pyrimidine; 5-ethyl-2-(4-(5-((4-(methylsulfonyl)piperazin-1-yl)methyl)biphenyl-2-yloxy)piperidin-1-yl)pyrimidine; 5-ethyl-2-(4-(4-((4-(methylsulfonyl)piperazin-1-yl)methyl)-2-(1H-pyrazol-5-yl)phenoxy)piperidin-1-yl)pyrimidine; (E)-2-(4-(2-(5-chloropent-1-enyl)-4-((4-(methylsulfonyl)piperazin-1-yl)methyl)phenoxy)piperidin-1-yl)-5-ethylpyrimidine; (E)-4-(2-(1-(5-Ethylpyrimidin-2-yl)piperidin-4-yloxy)-5-((4-(methylsulfonyl)piperazin-1-yl)methyl)phenyl)but-3-en-1-ol; 3-(2-(1-(5-Ethylpyrimidin-2-yl)piperidin-4-yloxy)-5-((4-(methylsulfonyl)piperazin-1-yl)methyl)phenyl)propan-1-ol; 2-(1-(5-Ethylpyrimidin-2-yl)piperidin-4-yloxy)-N-(2-methoxyethyl)-5-((4-(methylsulfonyl)piperazin-1-yl)methyl)aniline; 4-(2-(1-(5-Ethylpyrimidin-2-yl)piperidin-4-yloxy)-5-((4-(methylsulfonyl)piperazin-1-yl)methyl)phenyl)morpholino; 2-(4-(2-Chloro-4-((1-(methylsulfonyl)piperidin-4-yl)methyl)phenoxy)piperidin-1-yl)-5-ethylpyrimidine; 3-(4-(3-Chloro-4-(1-(5-ethylpyrimidin-2-yl)piperidin-4-yloxy)benzyl)piperidin-1-ylsulfonyl)propan-1-ol; 2-(4-(2-chloro-4-((4-(methylsulfonyl)piperidin-1-yl)methyl)phenoxy)piperidin-1-yl)-5-ethylpyrimidine; 2-(4-(2-Chloro-4-((2-methyl-4-(methylsulfonyl)piperazin-1-yl)methyl)phenoxy)piperidin-1-yl)-5-ethylpyrimidine; 2-(4-(2-Chloro-4-((2-methyl-4-(methylsulfonyl)piperazin-1-yl)methyl)phenoxy)piperidin-1-yl)-5-ethylpyrimidine; N-(3-Chloro-4-(1-(5-ethylpyrimidin-2-yl)piperidin-4-yloxy)phenyl)-N-methyl-1-(methylsulfonyl)piperidin-4-amine; 4-(3-Chloro-4-(1-(5-ethylpyrimidin-2-yl)piperidin-4-yloxy)benzyl)piperazine-1-carbonitrile; 2-(4-(2-Chloro-4-((4-(2-methyl-2H-tetrazol-5-yl)piperazin-1-yl)methyl)phenoxy)piperidin-1-yl)-5-ethylpyrimidine; 1-(3-Chloro-4-(1-(5-ethylpyrimidin-2-yl)piperidin-4-yloxy)benzyl)piperidine-4-carbonitrile; 2-(4-(2-chloro-4-((4-(2-methyl-2H-tetrazol-5-yl)piperidin-1-yl)methyl)phenoxy)piperidin-1-yl)-5-ethylpyrimidine; 2-(4-(2-Chloro-4-((1-(methylsulfonyl)azetidin-3-yloxy)methyl)phenoxy)piperidin-1-yl)-5-ethylpyrimidine; 2-(4-(2-Chloro-4-((1-(methylsulfonyl)azetidin-3-yloxy)methyl)phenoxy)piperidin-1-yl)-5-ethylpyrimidine; and N-(1-(3-Chloro-4-(1-(5-ethylpyrimidin-2-yl)piperidin-4-yloxy)benzyl)azetidin-3-yl)-N-methylmethanesulfonamide.

Further compounds of the invention are detailed in the Examples and Table I, infra.

### Pharmacology and Utility

Compounds of the invention modulate the activity of GPR119 and, as such, are useful for treating diseases or disorders in which the activity of GPR119 contributes to the pathology and/or symptomology of the disease. This invention further provides compounds of this invention for use in the preparation of medicaments for the treatment of diseases or disorders in which GPR119 activity contributes to the pathology and/or symptomology of the disease.

The resultant pathologies of Type II diabetes are impaired insulin signaling at its target tissues and failure of the insulin-producing cells of the pancreas to secrete an appropriate degree of insulin in response to a hyperglycemic signal. Current therapies to treat the latter include inhibitors of the β-cell ATP-sensitive potassium channel to trigger the release of endogenous insulin stores, or administration of exogenous insulin. Neither of these achieves accurate normalization of blood glucose levels and both carry the risk of inducing hypoglycemia. For these reasons, there has been intense interest in the development of pharmaceuticals that function in a glucose-dependent action, i.e. potentiators of glucose signaling. Physiological signaling systems which function in this manner are well-characterized and include the gut peptides GLP-1, GIP and PACAP. These hormones act via their cognate G-protein coupled receptor to stimulate the production of cAMP in pancreatic β-cells. The increased cAMP does not appear to result in stimulation of insulin release during the fasting or pre-prandial state. However, a series of biochemical targets of cAMP signaling, including the ATP-sensitive potassium channel, voltage-sensitive potassium channels and the exocytotic machinery, are modified in such a way that the insulin secretory response to a postprandial glucose stimulus is markedly enhanced. Accordingly, agonists of novel, similarly functioning, β-cell GPCRs, including GPR119, would also stimulate the release of endogenous insulin and consequently promote normoglycemia in Type II diabetes. It is also established that increased cAMP, for example as a result of GLP-1 stimulation, promotes β-cell proliferation, inhibits β-cell death and thus improves islet mass. This positive effect on β-cell mass is expected to be beneficial in both Type II diabetes, where insufficient insulin is produced, and Type I diabetes, where β-cells are destroyed by an inappropriate autoimmune response.

Some β-cell GPCRs, including GPR119, are also present in the hypothalamus where they modulate hunger, satiety, decrease food intake, controlling or decreasing weight and energy expenditure. Hence, given their function within the hypothalamic circuitry, agonists or inverse agonists of these receptors mitigate hunger, promote satiety and therefore modulate weight.

It is also well-established that metabolic diseases exert a negative influence on other physiological systems. Thus, there is often the codevelopment of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which clearly occur secondary to diabetes (e.g. kidney disease, peripheral neuropathy). Thus, it is expected that effective treatment of the diabetic condition will in turn be of benefit to such interconnected disease states.

In an embodiment of the invention is a method for treatment of a metabolic disease and/or a metabolic-related disorder in an individual comprising administering to the individual in need of such treatment a therapeutically effective amount of a compound of the invention or a pharmaceutical composition thereof. The metabolic diseases and metabolic-related disorders are selected from, but not limited to, hyperlipidemia, type 1 diabetes, type 2 diabetes mellitus, idiopathic type 1 diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.

In an embodiment of the invention are therapeutic benefits of GPR119 activity modulators derived from increasing levels of GIP and PPY. For example, neuroprotection, learning and memory, seizures and peripheral neuropathy.

GLP-1 and GLP-1 receptor agonists have been shown to be effective for treatment of neurodegenerative diseases and other neurological disorders. GLP-1 and exendin-4 have been shown to stimulate neurite outgrowth and enhance cell survival after growth factor withdrawal in PC12 cells. In a rodent model of neurodegeneration, GLP-1 and exendin-4 restore cholinergic marker activity in the basal forebrain. Central infusion of GLP-1 and exendin-4 also reduce the levels of amyloid-β peptide in mice and decrease amyloid precursor protein amount in cultured PC12 cells. GLP-1 receptor agonists have been shown to enhance learning in rats and the GLP-1 receptor knockout mice show deficiencies in learning behavior. The knockout mice also exhibit increased susceptibility to kainate-induced seizures which can be prevented by administration of GLP-1 receptor agonists. GLP-1 and exendin-4 has also been shown to be effective in treating pyridoxine-induced peripheral nerve degeneration, an experimental model of peripheral sensory neuropathy.

Glucose-dependent insulinotropic polypeptide (GIP) has also been shown to have effects on proliferation of hippocampal progenitor cells and in enhancing sensorimotor coordination and memory recognition.

In an embodiment of the invention are therapeutic benefits of GPR119 activity modulators. For example, GLP-2 and short bowel syndrome (SBS). Several studies in animals and from clinical trials have shown that GLP-2 is a trophic hormone that plays an important role in intestinal adaptation. Its role in regulation of cell proliferation, apoptosis, and nutrient absorption has been well documented. Short bowel syndrome is characterized by malabsorption of nutrients, water and vitamins as a result of disease or surgical removal of parts of the small intestine (eg. Crohn's disease). Therapies that improve intestinal adaptation are thought to be beneficial in treatment of this disease. In fact, phase II studies in SBS patients have shown that teduglutide, a GLP-2 analog, modestly increased fluid and nutrient absorption.

In an embodiment of the invention are therapeutic benefits of GPR119 activity modulators derived from increasing levels of GIP and PPY. For example, GLP-1, GIP and osteoporosis. GLP-1 has been shown to increase calcitonin and calcitonin related gene peptide (CGRP) secretion and expression in a murine C-cell line (CA-77). Calcitonin inhibits bone resorption by osteoclasts and promotes mineralization of skeletal bone. Osteoporosis is a disease that is characterized by reduced bone mineral density and thus GLP-1 induced increase in calcitonin might be therapeutically beneficial.

GIP has been reported to be involved in upregulation of markers of new bone formation in osetoblasts including collagen type I mRNA and in increasing bone mineral density. Like GLP-1, GIP has also been shown to inhibit bone resorption.

In an embodiment of the invention are therapeutic benefits of GPR119 activity modulators derived from increasing levels of GIP and PPY. For example, PPY and gastric emptying. GPR119 located on the pancreatic polypeptide (PP) cells of the islets has been implicated in the secretion of PPY. PPY has been reported to have profound effects on various physiological processes including modulation of gastric emptying and gastrointestinal motility. These effects slow down the digestive process and nutrient uptake and thereby prevent the postprandial elevation of blood glucose. PPY can suppress food intake by changing the expression of hypothalamic feeding-regulatory peptides. PP-overexpressing mice exhibited the thin phenotype with decreased food intake and gastric emptying rate.

In accordance with the foregoing, the present invention further provides a method for preventing or ameliorating the symptamology of any of the diseases or disorders described above in a subject in need thereof, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

Compounds of the invention can be administered in therapeutically effective amounts in combination with one or more therapeutic agents (pharmaceutical combinations).

For example, synergistic effects can occur with other anti-obesity agents, anorectic agents, appetite suppressant and related agents. Diet and/or exercise can also have synergistic effects. Anti-obesity agents include, but are not limited to, apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agents, 33 adrenergic receptor agonists, dopamine agonists (for example, bromocriptine), melanocyte-stimulating hormone receptor analogs, cannabinoid 1 receptor antagonists [for example, compounds described in WO2006/047516), melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Where compounds of the invention are administered in conjunction with other therapies, dosages of the co-administered compounds will of course vary depending on the type of co-drug employed, on the specific drug employed, on the condition being treated and so forth.

A combined preparation or pharmaceutical composition can comprise a compound of the invention as defined above or a pharmaceutical acceptable salt thereof and at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPAR gamma agonist e.g. GI-262570; Diacylglycerol acetyltransferase (DGAT) inhibitors such as those disclosed in WO 2005044250, WO 2005013907, WO 2004094618 and WO 2004047755;

b) hypolipidemic agents such as 3-hydroxy-3-methyl-glutaryl coenzyme A (HMG-CoA) reductase inhibitors, e.g., lovastatin and related compounds such as those disclosed in U.S. Pat. No. 4,231,938, pitavastatin, simvastatin and related compounds such as those disclosed in U.S. Pat. Nos. 4,448,784 and 4,450,171, pravastatin and related compounds such as those disclosed in U.S. Pat. No. 4,346,227, cerivastatin, mevastatin and related compounds such as those disclosed in U.S. Pat. No. 3,983,140, velostatin, fluvastatin, dalvastatin, atorvastatin, rosuvastatin and related statin compounds disclosed in U.S. Pat. No. 5,753,675, rivastatin, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, and quinoline and pyridine derivatives disclosed in U.S. Pat. Nos. 5,506,219 and 5,691,322. In addition, phosphinic acid compounds useful in inhibiting HMG CoA reductase suitable for use herein are disclosed in GB 2205837; squalene synthase inhibitors; FXR (farnesoid X receptor) and LXR (liver X receptor) ligands; cholestyramine; fibrates; nicotinic acid and aspirin;

c) an anti-obesity agent or appetite regulating agent such as a CB 1 activity modulator, melanocortin receptor (MC4R) agonists, melanin-concentrating hormone receptor (MCHR) antagonists, growth hormone secretagogue receptor (GHSR) antagonists, galanin receptor modulators, orexin antagonists, CCK agonists, GLP-1 agonists, and other Pre-proglucagon-derived peptides; NPY1 or NPY5 antagonsist, NPY2 and NPY4 modulators, corticotropin releasing factor agonists, histamine receptor-3 (H3) modulators, aP2 inhibitors, PPAR gamma modulators, PPAR delta modulators, acetyl-CoA carboxylase (ACC) inihibitors, 11-β-HSD-1 inhibitors, adinopectin receptor modulators; beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5, 491,134, 5,776,983 and 5,488,064, a thyroid receptor beta modulator, such as a thyroid receptor ligand as disclosed in WO 97/21993 (U. Cal SF), WO 99/00353 (KaroBio) and GB98/284425 (KaroBio), a SCD-1 inhibitor as disclosed in WO2005011655, a lipase inhibitor, such as orlistat or ATL-962 (Alizyme), serotonin receptor agonists, (e.g., BVT-933 (Biovitrum)), monoamine reuptake inhibitors or releasing agents, such as fenfluramine, dexfenfluramine, fluvoxamine, fluoxetine, paroxetine, sertraline, chlorphentermine, cloforex, clortermine, picilorex, sibutramine, dexamphetamine, phentermine, phenylpropanolamine or mazindol, anorectic agents such as topiramate (Johnson & Johnson), CNTF (ciliary neurotrophic factor)/Axokine® (Regeneron), BDNF (brain-derived neurotrophic factor), leptin and leptin receptor modulators, phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine;

d) anti-hypertensive agents such as loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; beta-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; aldosterone synthase inhibitors; and dual ET/AII antagonist such as those disclosed in WO 00/01389.

e) a HDL increasing compound;

f) Cholesterol absorption modulator such as Zetia® and KT6-971;

g) Apo-A1 analogues and mimetics;

h) thrombin inhibitors such as Ximelagatran;

i) aldosterone inhibitors such as anastrazole, fadrazole, eplerenone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron;

n) an agent for treating tobacco abuse, e.g., nicotine receptor partial agonists, bupropion hypochloride (also known under the tradename Zyban®) and nicotine replacement therapies;

o) an agent for treating erectile dysfunction, e.g., dopaminergic agents, such as apomorphine), ADD/ADHD agents (e.g., Ritalin®, Strattera®, Concerta® and Adderall®);

p) an agent for treating alcoholism, such as opioid antagonists (e.g., naltrexone (also known under the tradename ReVia®) and nalmefene), disulfiram (also known under the tradename Antabuse®), and acamprosate (also known under the tradename Campral®)). In addition, agents for reducing alcohol withdrawal symptoms may also be co-administered, such as benzodiazepines, beta-blockers, clonidine, carbamazepine, pregabalin, and gabapentin (Neurontin®);

q) other agents that are useful including anti-inflammatory agents (e.g., COX-2 inhibitors) ; antidepressants (e.g., fluoxetine hydrochloride (Prozac®)); cognitive improvement agents (e.g., donepezil hydrochloride (Aircept®) and other acetylcholinesterase inhibitors); neuroprotective agents (e.g., memantine); antipsychotic medications (e.g., ziprasidone (Geodon®), risperidone (Risperdal®), and olanzapine (Zyprexa®));

or, in each case a pharmaceutically acceptable salt thereof; and optionally a pharmaceutically acceptable carrier.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

In the following schemes, several methods of preparing the compounds of the present invention are illustrative. One of skill in the art will appreciate that these methods are representative, and in no way inclusive of all methods for preparing the compounds of the present invention. The radicals in the schemes are as described in Formula I.

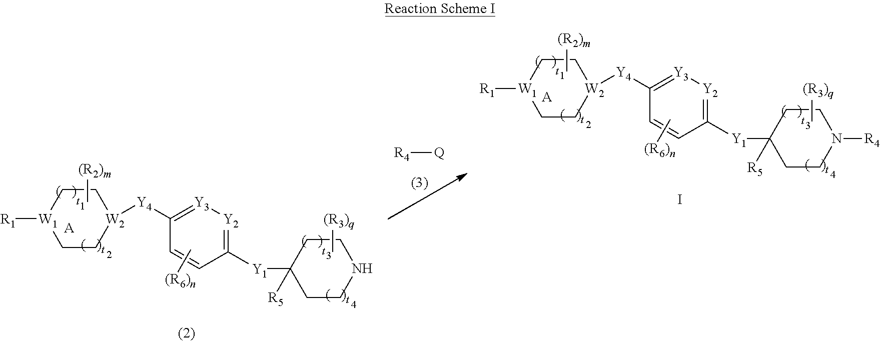

A compound of Formula I can be prepared by reacting a compound of formula 2 with a compound of formula 3, where Q is a leaving group (for example Cl, Br, OCF₃ and the like) in the presence of a suitable solvent (for example, dimethylacetamide, dimethylformamide, and the like) and a suitable base (for example, potassium carbonate, sodium t-butoxide, and the like), optionally in the presence of a palladium catalyst (for example, Pd₂(dba)₃, and the like) and a suitable ligand (for example, xantphos, and the like). The reaction proceeds at a temperature of about 0° C. to about 200° C. and can take up to 24 hours to complete.

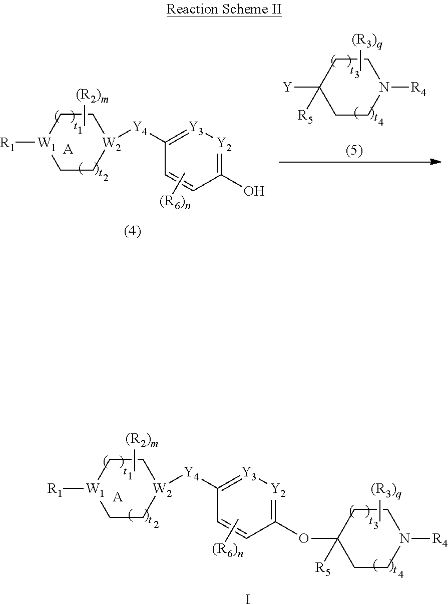

A compound of Formula I, where Y₁ is oxygen, can be prepared by reacting a compound of formula 4 with a compound of formula 5, where Y is a leaving group (for example OMs, Cl, Br, I and the like), in the presence of a suitable solvent (for example, dimethylformamide, acetonitrile, and the like) and a suitable base (for example, K₂CO₃, Cs₂CO₃, triethylamine and the like). The reaction proceeds at a temperature of about 0° C. to about 120° C. and can take up to 24 hours to complete.

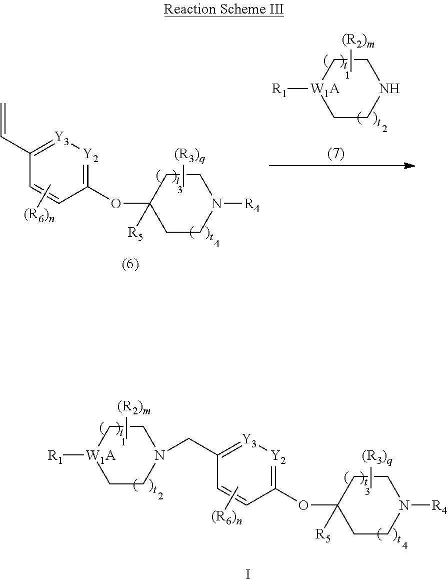

A compound of Formula I, where Y₄ is CH₂ and W₂ is N, can be prepared by reacting a compound of formula 6 with a compound of formula 7 in the presence of a suitable solvent (for example, dichloroethane, ethanol, and the like) followed by addition of a suitable reducing agent (for example, sodium cyanoborohydride, sodium triacetoxyborohydride, and the like). The reaction proceeds at a temperature of about 0° C. to about 120° C. and can take up to 24 hours to complete.

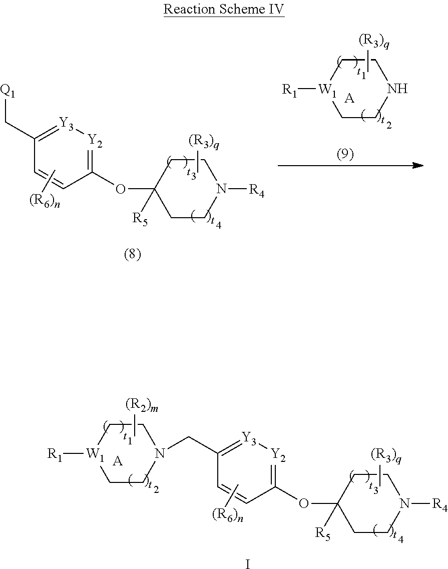

A compound of Formula I, where Y₄ is CH₂ and W₂ is N, can be prepared by reacting a compound of formula 8 with a compound of formula 9, where Q₁ is a leaving group (for example OMs, Cl, Br, I and the like), in the presence of a suitable solvent (for example, dimethylformamide, acetonitrile, and the like) and a suitable base (for example, K₂CO₃, Cs₂CO₃, triethylamine and the like). The reaction proceeds at a temperature of about 0° C. to about 120° C. and can take up to 24 hours to complete.

Detailed descriptions of the synthesis of compounds of the Invention are given in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction schemes I, II, III, IV; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following Examples that illustrate the preparation of compounds of the invention and their intermediates.

### Example A1

### 5-Ethyl-2-(4-(4-((4-(methylsulfonyppiperazin-1-yl)methypphenoxy)-piperidin-1-yl)pyrimidine

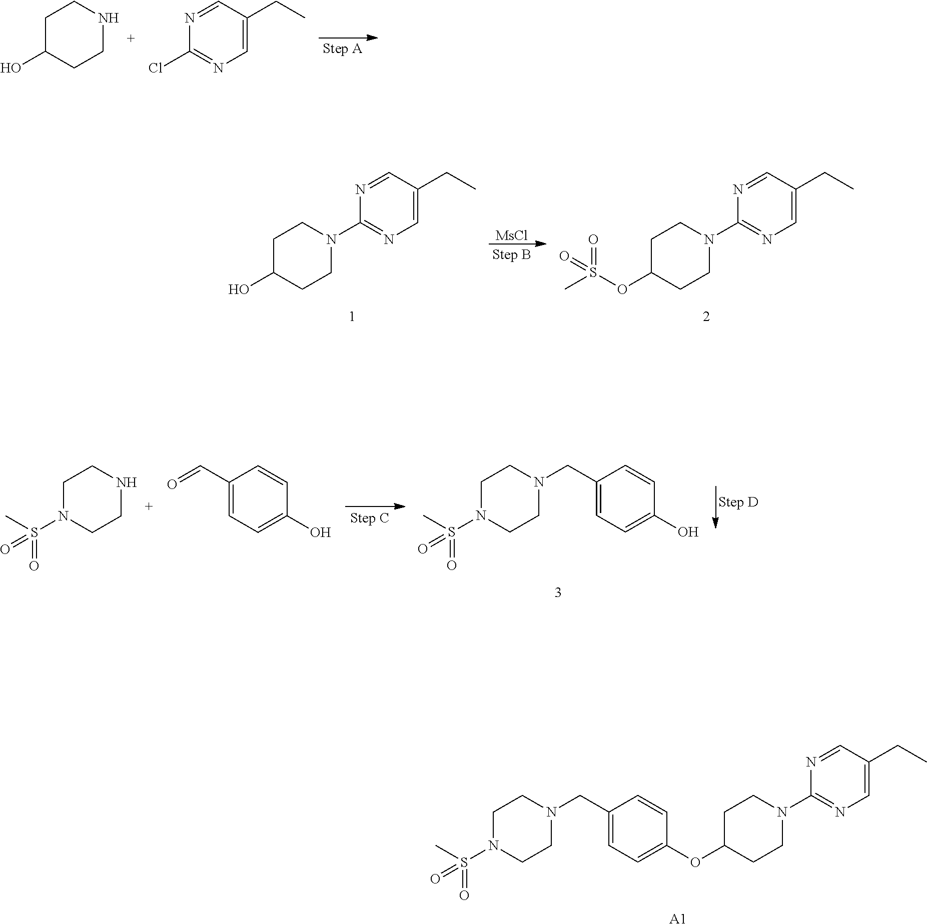

Step A: 4-Hydroxypiperidine (7 g, 70 mmol), 2-chloro-5-ethylpyrimidine (10 g, 70 mmol) and K₂CO₃ (14.5 g, 105 mmol) were dissolved in DMA (50 mL) and stirred at 150° C. for 12 h. The reaction mixture was cooled, diluted with H₂O (100 mL) and extracted with EtOAc (3×100 mL). The organic layers were combined, washed with H₂O (100 mL) and brine (100 mL), dried (MgSO₄), filtered and concentrated to provide 1-(5-ethylpyrimidin-2-yl)piperidin-4-ol 1 which was used without purification in Step B: MS m/z for (M+H)⁺ C₁₁H₁₈N₃O calc. 208.1, found 208.2.

Step B: 1-(5-Ethylpyrimidin-2-yl)piperidin-4-ol 1 was dissolved in DCM (200 mL). Diisopropylethylamine (25 mL, 140 mmol) was added, then the mixture was cooled to 0° C. Methanesulfonyl chloride (6.5 mL, 84 mmol) was added dropwise and the mixture was stirred for 1 h at rt. The mixture was poured into H₂O (100 mL) and the organic layer was separated. The organic layer was washed with sat. aq. NaHCO₃, dried (MgSO₄), filtered and concentrated. The product was recrystallized from EtOAc/hexanes to provide 1-(5-ethylpyrimidin-2-yl)piperidin-4-yl methanesulfonate 2 as an off-white powder: ¹H NMR (400 MHz, CDCl₃) δ=8.20 (s, 2H), 4.99 (m, 1H), 4.21 (m, 2H), 3.61 (m, 2H), 3.07 (s, 3H), 2.49 (q, J=7.6 Hz, 2H), 2.07 (m, 2H), 1.89 (m, 4H), 1.55 (m, 2H), 1.45 (d, J=6.8 Hz, 1H), 1.21 (t, J=7.6 Hz, 3H). MS m/z for (M+H)⁺ C₁₂H₂₀N₃O₃S calc. 286.1, found 286.2.

Step C: 4-Hydroxybenzaldehyde (500 mg, 4.09 mmol) and 1-methanesulfonylpiperazine (670 mg, 4.09 mmol) were dissolved in dichloroethane (10 mL). AcOH (50 μL) was added, and the mixture was heated at 80° C. for 1 h. Then sodium triacetoxyborohydride (1.7 g, 8.2 mmol) was added and the mixture was heated at 80° C. for another 2 h. The reaction was cooled, diluted with sat. aq. NaHCO₃ (50 mL), and extracted with DCM (30 mL). The organic layer was washed with brine, dried (MgSO₄), filtered, concentrated and purified by flash column chromatography (SiO₂, MeOH/DCM gradient) to give 4-((4-(methylsulfonyl)piperazin-1-yl)methyl)phenol 3 as a white powder: ¹H NMR (400 MHz, MeOD) δ=7.15 (d, J=8.4 Hz, 2H), 6.75 (d, J=8.4 Hz, 2H), 3.48 (s, 2H), 3.22 (t, J=4.8 Hz, 4H), 2.83 (s, 3H), 2.54 (t, J=4.8 Hz, 4H). MS m/z for (M+H)⁺ C₁₂H₁₉N₂O₃S calc. 271.1, found 271.1.

Step D: 1-(5-Ethylpyrimidin-2-yl)piperidin-4-yl methanesulfonate 2 (253 mg, 0.89 mmol), 4-((4-(methylsulfonyl)piperazin-1-yl)methyl)phenol 3 (200 mg, 0.74 mmol) and Cs₂CO₃ (482 mg, 1.48 mmol) were heated in AcN (10 mL) at 80° C. for 2 h. The reaction was cooled, filtered, concentrated and purified by flash column chromatography (SiO₂, MeOH/DCM gradient) to give A1: ¹H NMR (400 MHz, CDCl₃) δ=8.11 (s, 2H), 7.14 (d, J=8.8 Hz, 2H), 6.82 (d, J=8.4 Hz, 2H), 4.46 (m, 1H), 4.12 (m, 2H), 3.56 (m, 2H), 3.41 (s, 2H), 3.17 (t, J=4.8 Hz, 4H), 2.70 (s, 3H), 2.48 (t, J=4.8 Hz, 4H), 2.39 (q, J=7.6 Hz, 2H), 1.95 (m, 2H), 1.74 (m, 2H), 1.22 (t, J=7.6 Hz, 3H). MS m/z for (M+H)⁺ C₂₃H₃₄N₅O₃S calc. 460.2, found 460.2.

By repeating the procedure described above, using appropriate starting materials, the following compounds of Formula I, as identified in Table 1, were obtained.

TABLE 1

| Compound | | |
|---|---|---|
| # | Structure | NMR and/or ESMS |
| A2 | 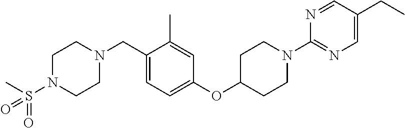 | ¹H NMR (400 MHz, CDCl₃) δ = 8.37 (s, 2H), 7.37 (d, J = 8.8 Hz, 1H), 6.84 (m, 2H), 4.66 (m, 1H), 4.22 (s, 2H), 4.06 (m, 2H), 3.95 (m, 2H), 2.89 (s, 3H), 2.56 (q, J = 7.6 Hz, 2H), 2.40 (s, 3H), 2.04 (m, 12H), 1.25 (t, J = 7.6 Hz, 3H). MS m/z for (M + H)⁺ C₂₄H₃₆N₅O₃S calc. 474.2, found 474.2. |
| A3 | 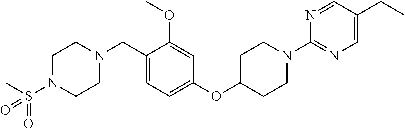 | ¹H NMR (400 MHz, CDCl₃) δ = 8.42 (s, 2H), 7.31 (d, J = 8.4 Hz, 1H), 6.57 (dd, J = 2.4, 8.4 Hz, 1H), 6.53 (d, J = 2.4 Hz, 1H), 4.68 (m, 1H), 4.24 (s, 2H), 4.03 (m, 4H), 3.87 (s, 3H), 3.58 (m, 2H), 3.44 (m, 2H), 2.98 (m, 2H), 2.88 (s, 3H), 2.60 (q, J = 7.6 Hz, 2H), 2.04 (m, 4H), 1.27 (t, J = 7.6 Hz, 3H). MS m/z for (M + H)⁺ C₂₄H₃₆N₅O₄S calc. 490.2, found 490.2. |
| A4 | 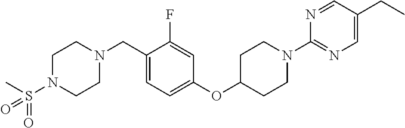 | ¹H NMR (400 MHz, CDCl₃) δ = 8.43 (s, 2H), 7.42 (t, J = 8.8 Hz, 1H), 6.81 (dd, J = 2.4, 8.8 Hz, 1H), 6.73 (dd, J = 2.4, 12.0 Hz, 1H), 4.66 (m, 1H), 4.25 (s, 2H), 4.06 (m, 4H), 2.88 (s, 3H), 2.59 (q, J = 7.6 Hz, 2H), 2.04 (m, 4H), 1.27 (t, J = 7.6 Hz, 3H). MS m/z for (M + H)⁺ C₂₃H₃₃FN₅O₃S calc. 478.2, found 478.2. |
| A5 | 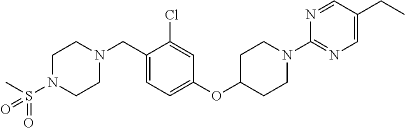 | ¹H NMR (400 MHz, CDCl₃) δ = 8.34 (s, 2H), 7.49 (d, J = 8.4 Hz, 1H), 6.96 (d, J = 2.8 Hz, 1H), 6.85 (dd, J = 2.8, 8.4 Hz, 1H), 4.60 (m, 1H), 4.30 (s, 2H), 3.95 (m, 6H), 2.80 (s, 3H), 2.51 (q, J = 7.6 Hz, 2H), 1.95 (m, 4H), 1.18 (t, J = 7.6 Hz, 3H). MS m/z for (M + H)⁺ C₂₃H₃₃ClN₅O₃S calc. 494.2, found 494.2. |
| A5 | 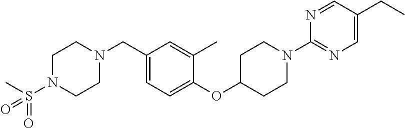 | ¹H NMR (400 MHz, CDCl₃) δ = = 8.44 (s, 2H), 7.21 (s, 1H), 7.20 (d, J = 8.8 Hz, 1H), 6.88 (d, J = 9.2 Hz, 1H), 4.74 (m, 1H), 4.17 (m, 2H), 4.14 (s, 2H), 3.94 (m, 2H), 3.59 (br. s, 2H), 3.47 (br. s, 2H), 2.89 (s, 3H), 2.60 (q, J = 7.6 Hz, 2H), 2.25 (s, 3H), 2.06 (m, 4H), 1.27 (t, J = 7.6 Hz, 3H). MS m/z for (M + H)⁺ C₂₄H₃₆N₅O₃S calc. 474.2, found 474.2. |
| A6 | 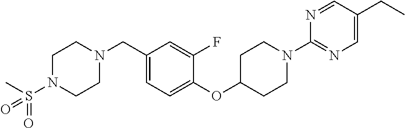 | ¹H NMR (400 MHz, MeOD) δ = = 8.39 (s, 2H), 7.35 (m, 3H), 4.82 (m, 1H), 4.36 (s, 2H), 4.10 (m, 2H), 3.85 (m, 2H), 2.95 (s, 3H), 2.59 (q, J = 7.6 Hz, 2H), 2.11 (m, 2H), 1.91 (m, 2H), 1.24 (t, J = 7.6 Hz, 3H). MS m/z for (M + H)⁺ C₂₃H₃₃FN₅O₃S calc. 478.2, found 478.2. |
| A7 | 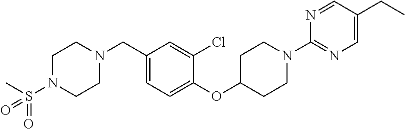 | ¹H NMR (400 MHz, CDCl₃) δ = 8.44 (s, 2H), 7.45 (d, J = 2.0 Hz, 1H), 7.35 (dd, J = 2.4, 8.4 Hz, 1H), 7.03 (d, J = 8.8 Hz), 4.80 (m, 1H), 4.24 (m, 2H), 4.15 (s, 2H), 3.94 (m, 2H), 2.90 (s, 3H), 2.59 (q, J = 7.6 Hz, 2H), 2.07 (m, 4H), 1.27 (t, J = 7.6 Hz, 3H). MS m/z for (M + H)⁺ C₂₃H₃₃ClN₅O₃S calc. 494.2, found 494.2. |
| A8 | 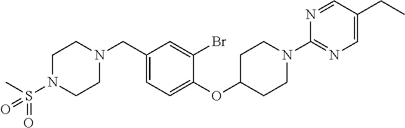 | ¹H NMR (400 MHz, MeOD) δ = 8.36 (s, 2H), 7.80 (d, J = 2.4 Hz, 1H), 7.50 (dd, J = 2.4, 8.8 Hz, 1H), 7.26 (d, J = 8.8 Hz), 4.90 (m, 1H), 4.35 (s, 2H), 3.99 (m, 4H), 2.96 (s, 3H), 2.58 (q, J = 7.6 Hz, 2H), 2.07 (m, 2H), 1.94 (m, 2H), 1.24 (t, J = 7.6 Hz, 3H). MS m/z for (M + H)⁺ C₂₃H₃₂BrN₅O₃S calc. 538.1, found 538.2. |
| A9 | 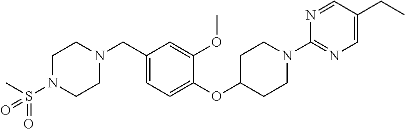 | ¹H NMR (400 MHz, MeOD) δ = 8.40 (s, 2H), 7.19 (d, J = 2.0 Hz, 1H), 7.15 (d, J = 8.4 Hz, 1H), 7.06 (dd, J = 2.0, 8.4 Hz, 1H), 4.72 (m, 1H), 4.35 (s, 2H), 4.10 (m, 2H), 3.88 (s, 3H), 3.84 (m, 2H), 2.95 (s, 3H), 2.59 (q, J = 7.6 Hz, 2H), 2.06 (m, 2H), 1.91 (m, 2H), 1.25 (t, J = 7.6 Hz, 3H). MS m/z for (M + H)⁺ C₂₄H₃₆N₅O₄S calc. 490.2, found 490.2. |
| A10 | 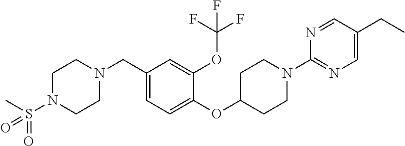 | ¹H NMR (400 MHz, MeOD) δ = 8.38 (s, 2H), 7.55 (d, J = 2.0 Hz, 1H), 7.51 (dd, J = 2.0, 8.4 Hz, 1H), 7.39 (d, J = 8.4 Hz, 1H), 4.39 (s, 2H), 4.04 (m, 2H), 3.90 (s, 2H), 2.96 (s, 3H), 2.59 (q, J = 7.6 Hz, 2H), 2.11 (m, 2H), 1.91 (m, 2H), 1.25 (t, J = 7.6 Hz, 3H). MS m/z for (M + H)⁺ C₂₄H₃₃F₃N₅O₄S calc. 544.2, found 544.2. |
| A11 | 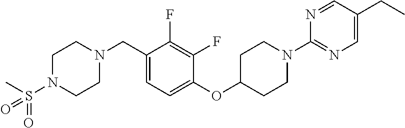 | ¹H NMR (400 MHz, MeOD) δ = 8.43 (s, 2H), 7.25 (t, J = 9.2 Hz, 1H), 6.90 (t, J = 8.8 Hz, 1H), 4.75 (m, 1H), 4.27 (s, 2H), 4.08 (m, 4H), 2.88 (s, 3H), 2.59 (q, J = 7.6 Hz, 2H), 2.07 (m, 4H), 1.27 (t, J = 7.6 Hz, 3H). MS m/z for (M + H)⁺ C₂₃H₃₂F₂N₅O₃S calc. 496.2, found 496.2. |
| A12 | 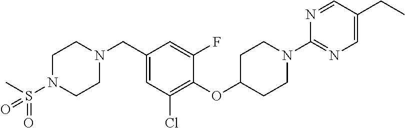 | ¹H NMR (400 MHz, MeOD) δ = 8.33 (s, 2H), 7.50 (t, J = 2.0 Hz, 1H), 7.39 (dd, J = 2.0, 11.2 Hz, 1H), 4.66 (m, 1H), 4.34 (s, 2H), 4.19 (m, 2H), 3.73 (m, 2H), 3.39 (m, 4H), 2.96 (s, 3H), 2.56 (q, J = 7.6 Hz, 2H), 2.05 (m, 2H), 1.92 (m, 2H), 1.23 (t, J = 7.6 Hz, 3H). MS m/z for (M + H)⁺ C₂₃H₃₂ClFN₅O₃S calc. 512.2, found 512.2. |
| A13 | 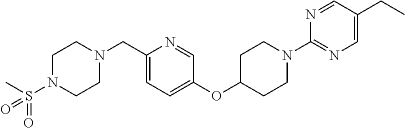 | ¹H NMR (400 MHz, CDCl₃) δ = 8.42 (s, 2H), 7.36 (d, J = 2.8 Hz, 1H), 7.52 (d, J = 8.4 Hz, 1H), 7.34 (dd, J = 2.8, 8.4 Hz, 1H), 4.74 (m, 1H), 4.30 (s, 2H), 4.04 (m, 4H), 3.64 (m, 4H), 3.39 (m, 4H), 2.88 (s, 3H), 2.59 (q, J = 7.6 Hz, 2H), 2.10 (m, 2H), 2.01 (m, 2H), 1.27 (t, J =7.6 Hz, 3H). MS m/z for (M + H)⁺ C₂₂H₃₃N₆O₃S calc. 461.2, found 461.2. |
| A14 | 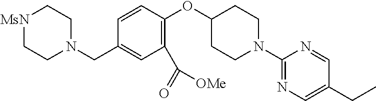 | ¹H NMR (400 MHz, CDCl₃) δ = 8.25 (s, 2H), 7.58 (d, J = 2.0 Hz, 1H), 7.43 (dd, J = 2.0, 8.8 Hz, 1H), 7.20 (d, J = 8.8 Hz, 1H), 4.77 (m, 1H), 3.89 (m, 2H), 3.77 (m, 4H), 3.47 (s, 2H), 3.33 (s, 3H), 3.09 (m, 4H), 2.86 (s, 3H), 2.44 (m, 6H), 1.87 (m, 2H), 1.66 (m, 2H), 1.13 (t, J = 7.6 Hz, 3H). MS m/z for (M + H)⁺ C₂₅H₃₆N₅O₅S calc. 518.2, found 518.2 |

### Example B1

### N-tert-Butyl 4-(4-((4-(methylsulfonyl)piperazin-1-yl)methyl)phenoxy)piperidine-1-carboxylate

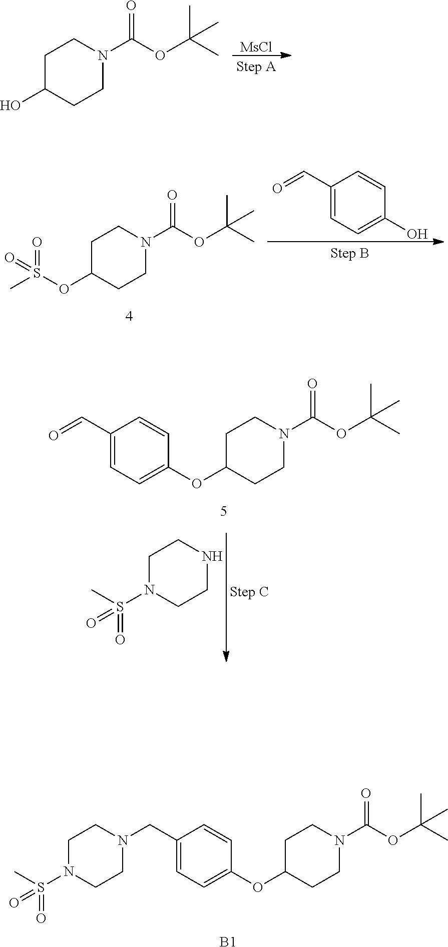

Step A: N-tert-Butyl 4-hydroxypiperidine-1-carboxylate (2 g, 9.94 mmol) was dissolved in DCM (20 mL). Diisopropylethylamine (3.4 mL, 19.8 mmol) was added and the mixture was cooled to 0° C., then methanesulfonyl chloride (0.92 mL, 11.93 mmol) was added dropwise and the mixture was stirred for 1 h at rt. The mixture was diluted with H₂O (20 mL) and separated. The organic layer was washed with sat. aq. NaHCO₃, dried (MgSO₄), filtered, concentrated and purified by flash column chromatography (SiO₂, MeOH/DCM gradient) to give N-tert-butyl 4-(methylsulfonyloxy)piperidine-1-carboxylate 4: ¹H NMR (400 MHz, CDCl₃) δ=5.00 (m, 1H), 3.82 (m, 2H), 3.42 (m, 2H), 3.16 (s, 3H), 2.08 (m, 2H), 1.94 (m, 2H), 1.58 (s, 9H). MS m/z for (M−C₄H₁₁+H⁺ fragment) C₇H₁₄NO₅S calc. 224.0, found 224.1.

Step B: N-tert-Butyl 4-(methylsulfonyloxy)piperidine-1-carboxylate 4 (500 mg, 1.79 mmol), 4-hydroxybenzaldehyde (218 mg, 1.79 mmol) and Cs₂CO₃ (1.1 g, 3.58 mmol) were heated in DMF (10 mL) at 80° C. for 2 h. The reaction was cooled, diluted with H₂O (20 mL) and extracted with EtOAc (2×20 mL). The organic layers were combined, washed with H₂O (20 mL) then brine (10 mL), dried (MgSO₄), filtered, concentrated and purified by flash column chromatography (SiO₂, EtOAc/Hexanes gradient) to give N-tert-butyl 4-(4-formylphenoxy)piperidine-1-carboxylate 5: ¹H NMR (400 MHz, CDCl₃) δ=7.77 (d, J=8.8 Hz, 2H), 6.94 (d, J=8.8 Hz, 2H), 4.54 (m, 1H), 3.63 (m, 2H), 3.32 (m, 2H), 1.89 (m, 2H), 1.73 (m, 2H), 1.39 (s, 9H). MS m/z for (M−C₄H₁₁+H⁺ fragment) C₁₃H₁₆NO₄ calc. 250.1, found 250.1.

Step C: N-tert-Butyl 4-(4-formylphenoxy)piperidine-1-carboxylate 5 (273 mg, 0.89 mmol) and 1-methanesulfonylpiperazine (176 mg, 1.07 mmol) were dissolved in dichloroethane (10 mL). AcOH (50 μL) was added, and the mixture was heated at 90° C. for 1 h, then sodium triacetoxyborohydride (725 mg, 3.42 mmol) was added and the mixture was heated at 90° C. for 2 h. The reaction was cooled, diluted with sat. aq. NaHCO₃ (50 mL), and extracted with DCM (30 mL). The organic layer was washed with brine, dried (MgSO₄), filtered, concentrated and purified by flash column chromatography (SiO₂, MeOH/DCM gradient) to give the title compound B1 as a white powder: ¹H NMR (400 MHz, MeOD) δ=7.22 (d, J=8.8 Hz, 2H), 6.88 (d, J=8.4 Hz, 2H), 4.47 (m, 1H), 3.72 (m, 2H), 3.50 (s, 2H), 3.25 (m, 4H), 2.79 (s, 3H), 2.56 (m, 4H), 1.92 (m, 2H), 1.76 (m, 2H), 1.49 (s, 9H). MS m/z for (M+H)⁺ C₂₂H₃₆N₃O₅S calc. 454.2, found 454.2.

### Example B2

### 1-Methylcyclopropyl 4-(4-((4-(methylsulfonyl)piperazin-1-yl)methyl)phenoxy)piperidine-1-carboxylate

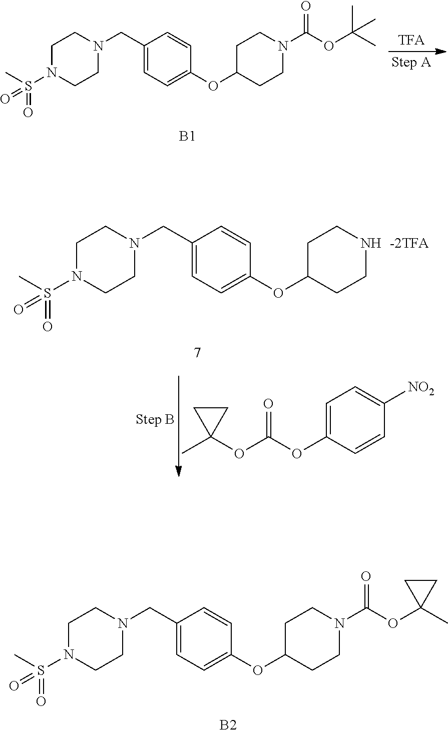

Step A: N-tert-Butyl 4-(4-((4-(methylsulfonyl)piperazin-1-yl)methyl)phenoxy)piperidine-1-carboxylate B1 (340 mg, 0.75 mmol) was dissolved in TFA (20 mL) and stirred at rt for 1 h. The mixture was concentrated in vacuo and the residue was dissolved in MeOH and concentrated (2×) to provide the TFA salt of 1-(methylsulfonyl)-4-(4-(piperidin-4-yloxy)benzyl)piperazine 7 (563 mg), which was used directly in Step B without further purification. MS m/z for (M−C₄H₁₁+⁺ fragment) C₁₇H₂₈N₃O₃S calc. 354.2, found 354.2.

Step B: 1-(Methylsulfonyl)-4-(4-(piperidin-4-yloxy)benzyl)piperazine 7 (36 mg, 0.06 mmol) and 1-methylcyclopropyl 4-nitrophenyl carbonate (22 mg, 0.09 mmol) were dissolved in DCM (5 mL). Triethylamine (60 μL, 0.43 mmol) was added and the mixture was stirred at rt for 2 h. The mixture was concentrated in vacuo and purified by flash column chromatography (SiO₂, EtOAc/Hexane gradient) to give the title compound B2: ¹H NMR (400 MHz, CDCl₃) δ=7.13 (d, J=8.4 Hz, 2H), 6.78 (d, J=8.4 Hz, 2H), 4.38 (m, 1H), 3.60 (m, 2H), 3.40 (s, 2H), 3.29 (m, 2H), 3.16 (m, 4H), 2.70 (s, 3H), 2.47 (m, 4H), 1.82 (m, 2H), 1.69 (m, 2H), 1.48 (s, 3H), 1.19 (m, 1H), 0.80 (m, 2H), 0.56 (m, 2H). MS m/z for (M+H)⁺ C₂₂H₃₄N₃O₅S calc. 452.2, found 452.2.

### Example B3

### 1-Methylcyclopropyl 4-(2-chloro-4-((4-(methylsulfonyl)piperazin-1-yl)methyl)phenoxy)piperidine-1-carboxylate

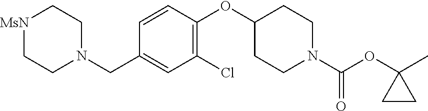

By following the same procedures as B1 except using 3-chloro-4-hydroxybenzaldehyde as the phenol, the title compound B3 was obtained; ¹H NMR (400 MHz, CDCl₃) δ7.42 (d, J=2.4 Hz, 1H), 7.32 (dd, J=2.4, 8.4 Hz, 1H), 6.98 (d, J=8.4 Hz, 1H), 4.63 (m, 1H), 4.15 (s, 2H), 3.59 (m, 4H), 2.88 (s, 3H), 1.87 (m, 4H), 1.56 (s, 3H), 0.89 (t, J=2.4 Hz, 2H), 0.65 (t, J=2.4 Hz, 2H); MS m/z for (M+H)⁺ C₂₂H₃₃ClN₃O₅S calc. 486.2, found 486.1.

### Example B4

### 5-Fluoro-2-(4-(4-((4-(methylsulfonyl)piperazin-1-yl)methyl)phenoxy)piperidin-1-yl)pyrimidine

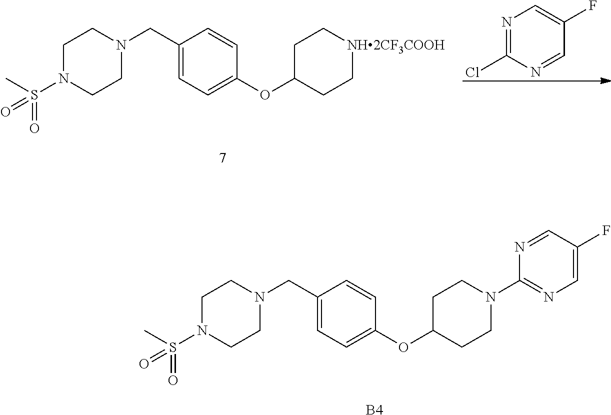

1-(Methylsulfonyl)-4-(4-(piperidin-4-yloxy)benzyl)piperazine 7 (30 mg, 0.05 mmol), 2-chloro-5-fluoro-pyrimidine (7 μL, 0.06 mmol) and K₂CO₃ (24 mg, 0.17 mmol) were dissolved in DMA (1 mL) and subjected to microwave irradiation (180° C., 5 min). The reaction was cooled, diluted with H₂O (10 mL) and extracted with EtOAc (10 mL). The organic layer was washed with H₂O (10 mL) and brine (10 mL), then dried (MgSO₄), filtered, concentrated and purified by reverse-phase HPLC (H₂O/AcN gradient) to afford the title compound B4 as a white solid: ¹H NMR (400 MHz, CDCl₃) δ=8.52 (s, 2H), 7.31 (d, J=8.8 Hz, 2H), 6.91 (d, J=8.4 Hz, 2H), 4.52 (m, 1H), 4.08 (s, 2H), 4.03 (m, 2H), 3.79 (m, 2H) 3.63 (m, 2H), 3.48 (m, 4H), 2.80 (s, 3H), 1.95 (m, 2H), 1.76 (m, 2H). MS m/z for (M+H)⁺ C₂₁H₂₉FN₅O₃S calc. 450.2, found 450.2.

By repeating the procedures described above, using appropriate starting materials, the following compounds of Formula I, as identified in Table 2, were obtained.

TABLE 2

| Compound | | |
|---|---|---|
| # | Structure | NMR and/or ESMS |
| B5 | 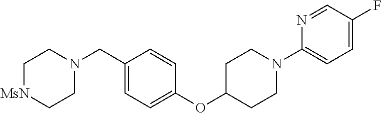 | ¹H NMR (400 MHz, CDCl₃) δ = 8.08 (t, J = 2.4 Hz, 1H), 7.60 (m, 1H), 7.33 (d, J = 8.4 Hz, 2H), 6.95 (dd, J = 3.6, 10 Hz, 1H), 6.92 (d, J = 8.8 Hz, 2H), 4.62 (s, 1H), 4.10 (s, 2H), 3.73 (m, 8H), 3.48 (m, 4H), 2.85 (m, 2H), 2.81 (s, 3H), 2.05 (m, 4H). MS m/z for (M + H)⁺ C₂₂H₃₀FN₄O₃S calc. 449.2, found 449.2. |
| B6 | 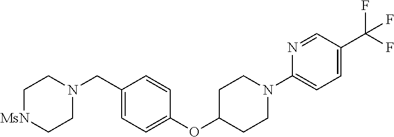 | ¹H NMR (400 MHz, CDCl₃) δ = 8.46 (s, 1H), 7.79 (dd, J = 2.0, 9.2 Hz, 1H), 7.39 (d, J = 8.4 Hz, 2H), 7.00 (d, J = 8.4 Hz, 2H), 6.89 (d, J = 9.6 Hz, 1H), 4.70 (m, 1H), 4.19 (s, 2H), 3.92 (m, 6H), 3.48 (m, 4H), 2.95 (m, 2H), 2.90 (s, 3H), 2.05 (m, 4H). MS m/z for (M + H)⁺ C₂₃H₃₀F₃N₄O₃S calc. 499.2, found 499.2. |
| B7 | 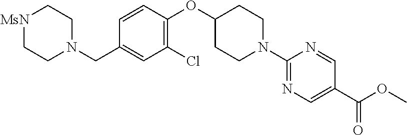 | ¹H NMR (400 MHz, CDCl₃) δ = 8.77 (s, 2H), 7.30 (d, J = 2.0 Hz, 1H), 7.06 (dd, J = 2.0, 8.4 Hz, 1H), 6.86 (d, J = 8.4 Hz, 1H), 4.58 (m, 1H), 4.02 (m, 4H), 3.80 (s, 3H), 3.40 (s, 2H), 3.18 (m, 4H), 2.72 (s, 3H), 2.48 (m, 4H), 1.90 (m, 4H). MS m/z for (M + H)⁺ C₂₃H₃₁ClN₅O₅S calc. 524.2, found 524.1. |
| B8 | 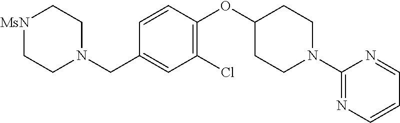 | ¹H NMR (400 MHz, CDCl₃) δ = 8.25 (s, 1H), 8.24 (s, 1H), 7.29 (d, J = 2.4 Hz, 1H), 7.05 (dd, J = 2.0, 8.4 Hz, 1H), 6.87 (d, J = 8.4 Hz, 1H), 6.41 (t, J = 7.2 Hz, 1H), 4.54 (m, 1H), 4.03 (m, 2H), 3.78 (m, 2H), 3.40 (s, 2H), 3.18 (m, 4H), 2.71 (s, 3H), 2.48 (m, 4H), 1.88 (m, 4H). MS m/z for (M + H)⁺ C₂₁H₂₉ClN₅O₃S calc. 466.2, found 466.1. |
| B9 | 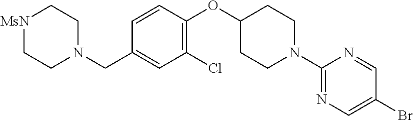 | ¹H NMR (400 MHz, CDCl₃) δ = 8.31 (s, 2H), 7.38 (d, J = 2.0 Hz, 1H), 7.14 (dd, J = 2.4, 8.4 Hz, 1H), 6.95 (d, J = 8.4 Hz, 1H), 4.63 (m, 1H), 4.03 (m, 2H), 3.88 (m, 2H), 3.48 (s, 2H), 3.27 (m, 4H), 2.80 (s, 3H), 2.57 (m, 4H), 1.96 (m, 4H). MS m/z for (M + H)⁺ C₂₁H₂₈BrClN₅O₃S calc. 544.1, found 544.1. |
| B10 | 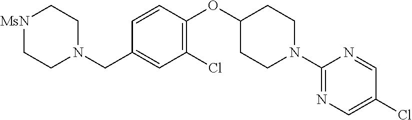 | ¹H NMR (400 MHz, CDCl₃) δ = 8.16 (s, 2H), 7.29 (d, J = 2.0 Hz, 1H), 7.05 (dd, J = 2.0, 8.4 Hz, 1H), 6.86 (d, J = 8.4 Hz, 1H), 4.55 (m, 1H), 3.93 (m, 2H), 3.80 (m, 2H), 3.40 (s, 2H), 3.18 (m, 4H), 2.72 (s, 3H), 2.49 (m, 4H), 1.87 (m, 4H). MS m/z for (M + H)⁺ C₂₁H₂₈Cl₂N₅O₃S calc. 500.1, found 500.2. |
| B11 | 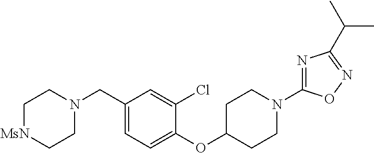 | ¹H NMR (400 MHz, CDCl₃) δ = 7.34 (d, J = 2.4 Hz, 1H), 7.23 (dd, J = 2.4, 8.0 Hz, 1H), 6.93 (d, J = 8.4 Hz, 1H), 4.65 (m, 1H), 4.02 (s, 3H), 3.70 (m, 5H), 3.60 (m, 3H), 2.82 (q, J = 7.2 Hz, 2H), 2.81 (s, 3H), 1.94 (m, 4H), 1.23 (s, 3H), 1.21 (s, 3H). MS m/z for (M + H)⁺ C₂₂H₃₃ClN₅O₄S calc. 498.2, found 498.1. |

### Example B12

### 2-(4-(2-Chloro-4-((4-(methylsulfonyl)piperazin-1-yl)methyl)phenoxy)piperidin-1-yl)pyrimidine-5-carboxamide

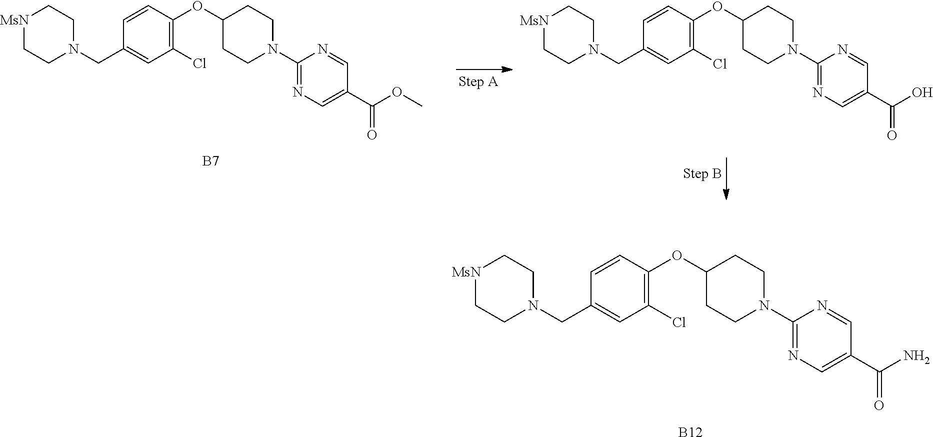

Step A: Methyl 2-(4-(2-chloro-4-((4-(methylsulfonyl)piperazin-1-yl)methyl)phenoxy)piperidin-1-yl)pyrimidine-5-carboxylate B7 (216 mg, 0.41 mmol) was dissolved in THF (5 mL) and LiOH (165 μL of 5M, 0.824 mmol) was added. The mixture was heated at 80° C. for 3 h, then cooled and concentrated to give crude 2-(4-(2-chloro-4-((4-(methylsulfonyl)piperazin-1-yl)methyl)phenoxy)piperidin-1-yl)pyrimidine-5-carboxylic acid, which was used in the next step without further purification. MS m/z for (M⁺+H⁺) C₂₂H₂₉ClN₅O₅S calc. 510.2, found 510.1.

Step B: 2-(4-(2-Chloro-4-((4-(methylsulfonyl)piperazin-1-yl)methyl)phenoxy)piperidin-1-yl)pyrimidine-5-carboxylic acid (21 mg, 0.41 mmol) was dissolved in a mixture of DCM (5 mL) and THF (5 mL). Oxalyl chloride (40 μL, 0.45 mmol) was added and the mixture was stirred at rt for 2 h, then ammonium hydroxide (100 μL of a 30% solution in H₂O) was added and the mixture was stirred at rt for 12 h. The mixture was concentrated and purified by reverse-phase HPLC (H₂O/AcN gradient) to afford the title compound B12 as a white solid: ¹H NMR (400 MHz, CDCl₃) δ=8.65 (s, 2H), 7.30 (d, J=2.0 Hz, 1H), 7.07 (dd, J=2.0, 8.4 Hz, 1H), 6.87 (d, J=8.4 Hz, 1H), 4.59 (m, 1H), 4.05 (m, 4H), 3.41 (m, 2H), 3.19 (m, 4H), 2.72 (s, 3H), 2.50 (m, 4H), 1.90 (m, 4H). MS m/z for (M+H)⁺ C₂₂H₃₀ClN₆O₄S calc. 509.2, found 509.2.

By repeating the procedures described above, using appropriate starting materials, the following compounds of Formula I, as identified in Table 3, were obtained.

TABLE 3

| | | |
|---|---|---|
| B13 | 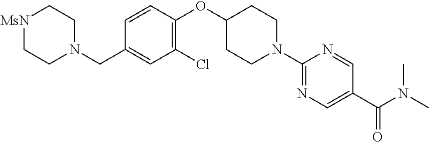 | ¹H NMR (400 MHz, CDCl₃) δ = 8.40 (s, 2H), 7.30 (d, J = 2.0 Hz, 1H), 7.06 (dd, J = 2.0, 8.4 Hz, 1H), 6.86 (d, J = 8.4 Hz, 1H), 4.57 (m, 1H), 3.97 (m, 4H), 3.40 (s, 2H), 3.18 (m, 4H), 3.03 (s, 6H), 2.71 (s, 3H), 2.48 (m, 4H), 1.90 (m, 4H). MS m/z for (M + H)⁺ C₂₄H₃₄ClN₆O₄S calc. 537.2, found 537.1. |
| B14 | 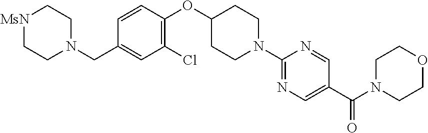 | ¹H NMR (400 MHz, CDCl₃) δ = 8.37 (s, 2H), 7.30 (d, J = 2.0 Hz, 1H), 7.06 (dd, J = 2.0, 8.4 Hz, 1H), 6.86 (d, J = 8.4 Hz, 1H), 4.57 (m, 1H), 3.97 (m, 4H), 3.65 (m, 4H), 3.59 (m, 4H), 3.40 (s, 2H), 3.18 (m, 4H), 2.72 (s, 3H), 2.48 (m, 4H), 1.89 (m, 4H). MS m/z for (M + H)⁺ C₂₆H₃₆ClN₆O₅S calc. 579.2, found 579.1. |

### Example B15

### 2-(4-(2-Chloro-4-((4-(methylsulfonyl)piperazin-1-yl)methyl)phenoxy)piperidin-1-yl)pyrimidine-5-carbonitrile

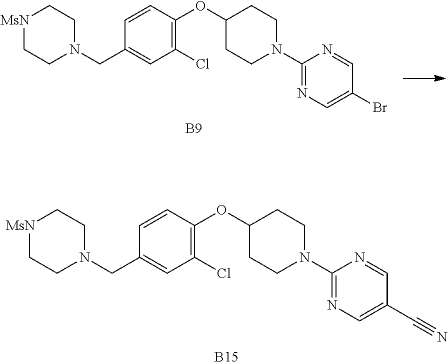

5-Bromo-2-(4-(2-chloro-4-((4-(methylsulfonyl)piperazin-1-yl)methyl)phenoxy)piperidin-1-yl)pyrimidine B9 (30 mg, 0.055 mmol) was dissolved in DMF (1 mL). Zn(CN)₂ (6.5 mg, 0.055 mmol) and Pd(PPh₃)₄ (13 mg, 0.011 mmol) were added, and the mixture was degassed with argon for 20 min, then heated at 65° C. for 12 h. The mixture was cooled, filtered and purified by reverse-phase HPLC (H₂O/AcN gradient) to afford the title compound B15 as a white solid: ¹H NMR (400 MHz, CDCl₃) δ=8.28 (s, 2H), 7.16 (d, J=2.0 Hz, 1H), 6.93 (dd, J=1.6, 8.4 Hz, 1H), 6.72 (d, J=8.4 Hz, 1H), 4.46 (m, 1H), 3.95 (m, 2H), 3.77 (m, 2H), 3.30 (s, 2H), 3.06 (m, 4H), 2.58 (s, 3H), 2.37 (m, 4H), 1.76 (m, 4H). MS m/z for (M+H)⁺ C₂₂H₂₈ClN₆O₃S calc. 491.2, found 491.2.

### Example B16

### 2-(4-(2-Chloro-4-((4-(methylsulfonyl)piperazin-1-yl)methyl)phenoxy)piperidin-1-yl)-5-(2H-tetrazol-5-yl)pyrimidine

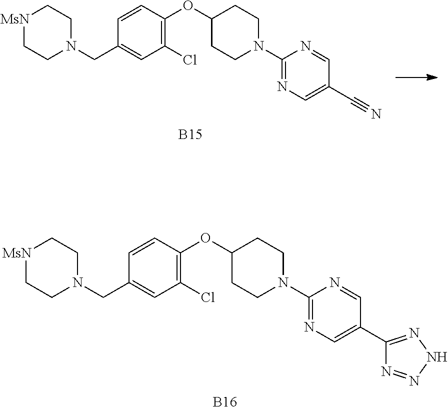

2-(4-(2-Chloro-4-((4-(methylsulfonyl)piperazin-1-yl)methyl)phenoxy)piperidin-1-yl)pyrimidine-5-carbonitrile B15 (100 mg, 0.20 mmol) was dissolved in DMF (3 mL). NaN₃ (53 mg, 0.81 mmol) and NH₄Cl (55 mg, 1.02 mmol) were added, and the mixture was heated at 90° C. for 12 h. The mixture was diluted with H₂O (10 mL), extracted with EtOAc (10 mL), washed with H₂O (10 mL) and brine (10 mL), dried (MgSO₄), filtered and purified by reverse-phase HPLC (H₂O/AcN gradient) to afford the title compound B16 as a white solid: ¹H NMR (400 MHz, DMSO-d₆) δ=8.93 (s, 2H), 7.53 (d, J=2.0 Hz, 1H), 7.35 (dd, J=2.0, 8.4 Hz, 1H), 7.19 (d, J=8.4 Hz, 1H), 4.81 (m, 1H), 4.18 (m, 2H), 3.92 (m, 4H), 3.42 (m, 4H), 3.03 (m, 4H), 2.85 (s, 3H), 2.06 (m, 2H), 1.85 (m, 2H). MS m/z for (M+H)⁺ C₂₂H₂₉ClN₉O₃S calc. 534.2, found 534.2.

### Example B17

### 2-(4-(2-Chloro-4-((4-(methylsulfonyl)piperazin-1-yl)methyl)phenoxy)piperidin-1-yl)-5-(2-methyl-2H-tetrazol-5-yl)pyrimidine

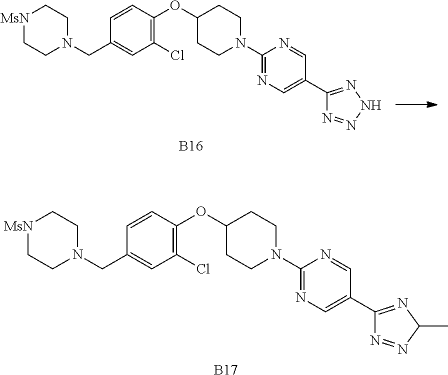

2-(4-(2-Chloro-4-((4-(methylsulfonyl)piperazin-1-yl)methyl)phenoxy)piperidin-1-yl)-5-(2H-tetrazol-5-yl)pyrimidine B16 (28 mg, 0.052 mmol) was dissolved in DMF (2 mL). K₂CO₃ (40 mg, 0.29 mmol) and MeI (4 μL, 0.06 mmol) were added and the mixture was stirred at rt for 2 h. The mixture was diluted with H₂O (10 mL), extracted with EtOAc (10 mL), washed with H₂O (10 mL) and brine (10 mL), dried (MgSO₄), filtered and purified by reverse-phase HPLC (H₂O/AcN gradient) to afford the title compound B17 as a white solid: ¹H NMR (400 MHz, DMSO-d₆) δ=8.92 (s, 2H), 7.30 (d, J=2.0 Hz, 1H), 7.06 (dd, J=2.0, 8.4 Hz, 1H), 6.88 (d, J=8.8 Hz, 1H), 4.58 (m, 1H), 4.32 (s, 3H), 4.04 (m, 2H), 3.96 (m, 2H), 3.40 (s, 2H), 3.18 (m, 4H), 2.72 (s, 3H), 2.48 (m, 4H), 1.91 (m, 4H). MS m/z for (M+H)⁺ C₂₃H₃₁ClN₉O₃S calc. 548.2, found 548.2.

### Example C1

### (E)-5-Ethyl-2-(4-(2-(3-methoxyprop-1-enyl)-4-((4-(methylsulfonyl)piperazin-1-yl)methyl)phenoxy)piperidin-1-yl)pyrimidine

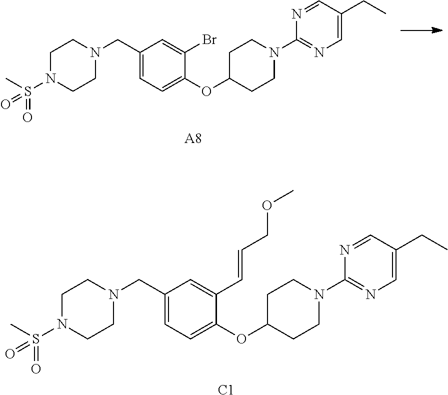

2-(4-(2-bromo-4-((4-(methylsulfonyl)piperazin-1-yl)methyl)phenoxy)piperidin-1-yl)-5-ethylpyrimidine A8 (60 mg, 0.11 mmol), (E)-2-(3-methoxy-1-propenyl)-4,4,5,5-tetramethyl-1,3,2-dioxoborolane (35 μL, 0.16 mmol), Na₂CO₃ (35 mg, 0.33 mmol) and Pd(PPh₃)₄ (13 mg, 0.011 mmol) were dissolved in a mixture of dimethoxyethane (360 μL), H₂O (240 μL) and EtOH (180 μL) and subjected to microwave irradiation (170° C., 5 min). The mixture was cooled, diluted with H₂O (10 mL) and extracted with EtOAc (20 mL). The organic layer was dried (MgSO₄), filtered, concentrated and purified by reverse-phase HPLC (H₂O/AcN gradient) to afford the title compound C1 as a white solid: ¹H NMR (400 MHz, CDCl₃) δ=8.43 (s, 2H), 7.54 (d, J=2.0 Hz, 1H), 7.26 (dd, J=2.4, 8.8 Hz, 1H), 6.94 (d, J=8.4 Hz, 1H), 6.88 (d, J=16 Hz, 1H), 6.32 (dt, J=7.6, 16 Hz, 1H), 4.73 (m, 1H), 4.16 (m, 2H), 4.10 (m, 2H), 4.02 (m, 3H), 3.40 (s, 3H), 2.89 (s, 3H), 2.68 (m, 7H), 2.59 (q, J=7.6 Hz, 2H), 2.06 (m, 4H), 1.26 (t, J=7.6 Hz, 3H); MS m/z for (M+H)⁺ C₂₇H₄₀N₅O₄S calc. 530.3, found 530.2.

### Example C2

### 3-(2-(1-(5-Ethylpyrimidin-2-yl)piperidin-4-yloxy)-5-((4-(methylsulfonyl)piperazin-1-yl)methyl)phenyl)propan-1-ol

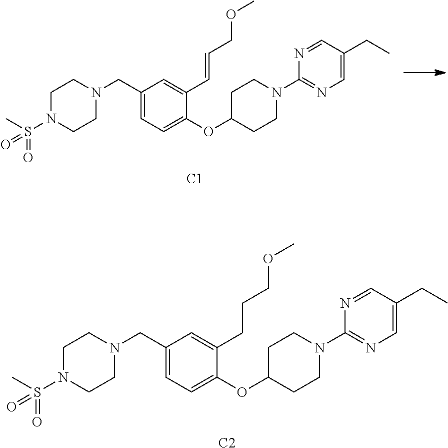

(E)-5-Ethyl-2-(4-(2-(3-methoxyprop-1-enyl)-4-((4-(methylsulfonyl)piperazin-1-yl)methyl)phenoxy)piperidin-1-yl)pyrimidine C2 (55 mg, 0.104 mmol) and Pd/C (˜20 mg of 10%, wet) was suspended in a flask with EtOH (10 mL) and the mixture was shaken under H₂ on a Parr shaker (55 psi) for 1 h. The mixture was filtered through celite, concentrated, and purified by reverse-phase HPLC (H₂O/AcN gradient) to afford the title compound C2 as a white solid: ¹H NMR (400 MHz, CDCl₃) δ=8.12 (s, 2H), 7.03 (d, J=2.0 Hz, 1H), 7.00 (dd, J=2.0, 8.4 Hz, 1H), 6.75 (d, J=8.4 Hz, 1H), 4.51 (m, 1H), 3.97 (m, 2H), 3.71 (m, 2H), 3.40 (s, 2H), 3.31 (t, J=6.8 Hz, 2H), 3.25 (s, 3H), 3.17 (s, 3H), 2.71 (s, 3H), 2.60 (m, 1H), 2.48 (s, 3H), 2.40 (q, J=7.6 Hz, 2H), 1.93 (m, 2H), 1.79 (m, 4H), 1.13 (t, J=7.6 Hz, 3H); MS m/z for (M+H)⁺ C₂₇H₄₂N₅O₄S calc. 532.3, found 532.0.

By repeating the procedures described above, using appropriate starting materials, the following compounds of Formula I, as identified in Table 4, were obtained.

TABLE 4

| | | |
|---|---|---|
| C3 | 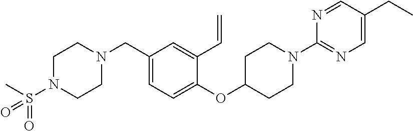 | ¹H NMR (400 MHz, CDCl₃) δ = 8.45 (s, 2H), 7.55 (d, J = 2.4 Hz, 1H), 7.28 (m, 1H), 7.01 (dd, J = 11.2, 17.6 Hz, 1H), 6.95 (d, J = 8.4 Hz, 1H), 5.77 (dd, J = 0.8, 17.6 Hz, 1H), 5.37 (dd, J = 1.2, 11.2 Hz, 1H), 4.77 (m, 1H), 4.17 (m, 4H), 3.95 (m, 2H), 3.62, (m, 2H), 3.46 (m, 2H), 2.89 (s, 3H), 2.61 (q, J = 7.6 Hz, 2H), 2.08 (m, 4H), 1.28 (t, J = 7.6 Hz, 3H); MS m/z for (M + H)⁺ C₂₅H₃₆N₅O₃S calc. 486.2, found 486.2. |
| C4 | 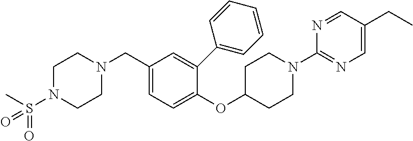 | ¹H NMR (400 MHz, CDCl₃) δ = 8.38 (s, 2H), 7.51 (m, 2H), 7.37 (m, 5H), 7.06 (d, J = 8.0 Hz, 1H), 4.73 (m, 1H), 4.22 (s, 2H), 4.07 (m, 2H), 3.60 (m, 3H), 2.88 (s, 3H), 2.56 (q, J = 7.6 Hz, 2H), 1.95 (m, 4H), 1.24 (t, J = 7.6 Hz, 3H); MS m/z for (M + H)⁺ C₂₉H₃₈N₅O₃S calc. 536.3, found 536.2. |
| C5 | 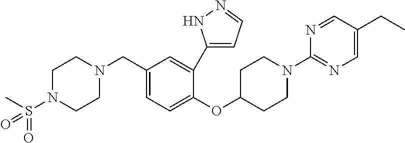 | ¹H NMR (400 MHz, CDCl₃) δ = 8.34 (s, 1H), 8.32 (s, 1H), 7.83 (d, J = 2.0 Hz, 0.5H), 7.68 (d, J = 2.0 Hz, 0.5H), 7.37 (dd, J = 2.0, 8.8 Hz, 0.5H), 7.24 (d, J = 8.8 Hz, 1H), 7.07 (d, J = 8.8 Hz, 0.5H), 6.90 (dd, J = 2.0, 6.8 Hz, 1H), 6.79 (d, J = 2.4 Hz, 0.5H), 4.79 (m, 0.5H), 4.61 (m, 0.5H), 4.15 (m, 2H), 4.08 (m, 2H), 3.95 (m, 3H), 3.69 (m, 2H), 2.81 (s, 1.5H), 2.80 (s, 1.5H), 2.50 (q, J = 7.6 Hz, 2H), 2.00 (m, 4H), 1.18 (t, J = 7.6 Hz, 3H); MS m/z for (M + H)⁺ C₂₆H₃₆N₇O₃S calc. 526.3, found 526.2. |
| C6 | 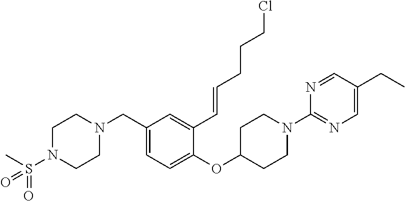 | ¹H NMR (400 MHz, CDCl₃) δ = 8.32 (s, 2H), 7.54 (d, J = 2.4 Hz, 1H), 7.19 (dd, J = 2.0, 8.4 Hz, 1H), 6.93 (d, J = 8.4 Hz, 1H), 6.74 (d, J = 16 Hz, 1H), 6.20 (dt, J = 8.8, 16 Hz, 1H), 4.67 (m, 1H), 4.14 (s, 2H), 4.04 (m, 2H), 3.94 (m, 3H), 3.59 (t, J = 6.4 Hz, 2H), 2.89 (s, 3H), 2.54 (m, 2H), 2.40 (m, 2H), 2.04 (m, 2H), 1.96 (m, 4H), 1.24 (t, J = 7.6 Hz, 3H); MS m/z for (M + H)⁺ C₂₈H₄₁ClN₅O₃S calc. 562.3, found 562.2. |

### Example C7

### (E)-4-(2-(1-(5-Ethylpyrimidin-2-yl)piperidin-4-yloxy)-5-((4-(methylsulfonyl)piperazin-1-yl)methyl)phenyl)but-3-en-1-ol

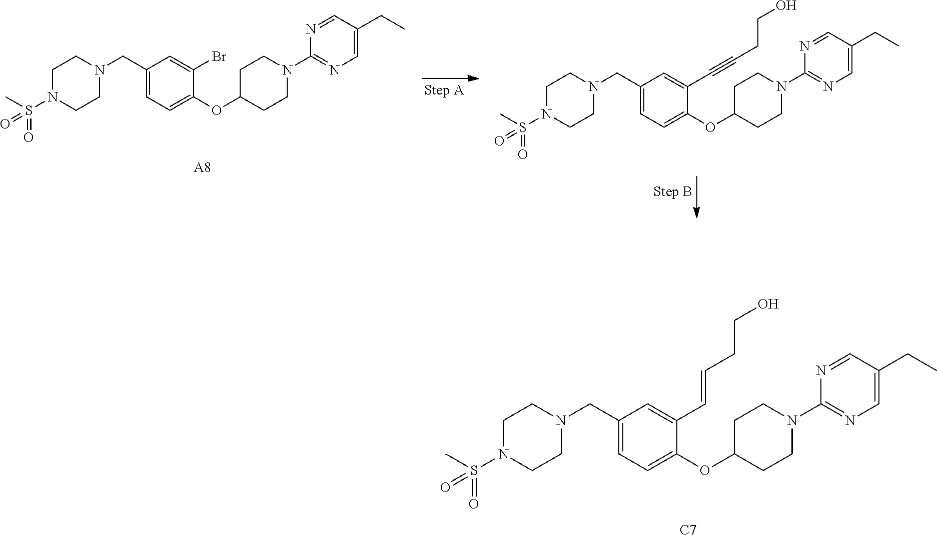

Step A: 2-(4-(2-Bromo-4-((4-(methylsulfonyl)piperazin-1-yl)methyl)phenoxy)piperidin-1-yl)-5-ethylpyrimidine A8 (60 mg, 0.11 mmol), but-3-yn-1-ol (182 μL, 3.12 mmol), CuBr (33 mg, 0.23 mmol) and Pd(PPh₃)₄ (25 mg, 0.022 mmol) were dissolved in triethylamine (750 μL), degassed for 20 min with argon, then heated at 90° C. for 3 h. The mixture was cooled, diluted with H₂O (10 mL) and extracted with EtOAc (20 mL). The organic layer was dried (MgSO₄), filtered, concentrated and purified by flash column chromatography (SiO₂, EtOAc/Hexane gradient) to give 4-(2-(1-(5-ethylpyrimidin-2-yl)piperidin-4-yloxy)-5-((4-(methylsulfonyl)piperazin-1-yl)methyl)phenyl)but-3-yn-1-ol as a white powder: ¹H NMR (400 MHz, CDCl₃) δ=8.11 (s, 2H), 7.27 (d, J=2.4 Hz, 1H), 7.10 (dd, J=2.4, 8.4 Hz, 1H), 6.81 (d, J=8.4 Hz, 1H), 4.54 (m, 1H), 4.07 (m, 2H), 3.66 (m, 4H), 3.38 (s, 2H), 3.17 (m, 4H), 2.71 (s, 3H), 2.63 (t, J=6.0 Hz, 2H), 2.47 (m, 4H), 2.40 (q, J=7.6 Hz, 2H), 1.94 (m, 4H), 1.82 (m, 2H), 1.53 (m, 2H), 1.12 (t, J=7.6 Hz, 3H); MS m/z for (M+H)⁺ C₂₇H₃₈N₅O₄S calc. 528.3, found 528.2.

Step B: 4-(2-(1-(5-Ethylpyrimidin-2-yl)piperidin-4-yloxy)-5-((4-(methylsulfonyl)piperazin-1-yl)methyl)phenyl)but-3-yn-1-ol (50 mg, 0.095 mmol) and cyclohexadiene (100 μL) were dissolved in EtOH (2 mL). Pd/C (20 mg of 10%, wet) was added and the mixture was heated at 80° C. overnight, then it was cooled and filtered through an 0.2 μm syringe filter. The mixture was concentrated and purified by reverse-phase HPLC (H₂O/AcN gradient) to afford the title compound C7 as a white solid: ¹H NMR (400 MHz, CDCl₃) δ=8.12 (s, 2H), 7.31 (d, J=2.4 Hz, 1H), 7.04 (dd, J=2.4, 8.4 Hz, 1H), 6.80 (d, J=8.4 Hz, 1H), 6.75 (d, J=16 Hz, 1H), 6.15 (dt, J=7.2, 16 Hz, 1H), 4.49 (m, 1H), 4.06 (m, 2H), 3.64 (m, 4H), 3.40 (s, 2H), 3.17 (m, 4H), 2.71 (s, 3H), 2.48 (m, 4H), 2.40 (m, 4H), 1.95 (m, 4H), 1.79 (m, 2H), 1.53 (m, 2H), 1.12 (t, J=7.6 Hz, 3H); MS m/z for (M+H)⁺ C₂₇H₄₀N₅O₄S calc. 530.3, found 530.2.

### Example C8

### 3-(2-(1-(5-Ethylpyrimidin-2-yl)piperidin-4-yloxy)-5-((4-(methylsulfonyl)piperazin-1-yl)methyl)phenyl)propan-1-ol

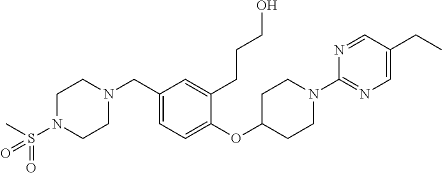

By following the same procedure as in C7 except using prop-2-yn-1-ol as the alkyne, the title compound C8 was obtained; ¹H NMR (400 MHz, CDCl₃) δ=8.11 (s, 2H), 7.04 (d, J=2.0 Hz, 1H), 7.01 (dd, J=2.0, 8.4 Hz, 1H), 6.77 (d, J=8.0 Hz, 1H), 4.51 (m, 1H), 4.07 (m, 2H), 3.61 (m, 2H), 3.53 (m, 2H), 3.40 (s, 2H), 3.17 (m, 4H), 2.71 (s, 3H), 2.65 (m, 2H), 2.48 (m, 4H), 2.40 (q, J=7.6 Hz, 2H), 1.97 (m, 3H), 1.77 (m, 4H), 1.52 (m, 2H), 1.13 (t, J=7.6 Hz, 3H); MS m/z for (M+H)⁺ C₂₆H₄₀N₅O₄S calc. 518.3, found 518.2.

### Example C9

### 2-(1-(5-Ethylpyrimidin-2-yl)piperidin-4-yloxy)-N-(2-methoxyethyl)-5-((4-(methylsulfonyl)piperazin-1-yl)methyl)aniline

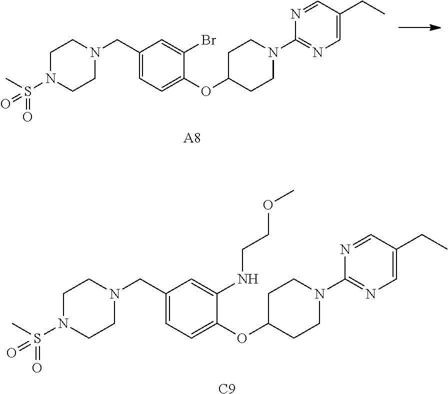

2-(4-(2-Bromo-4-((4-(methylsulfonyl)piperazin-1-yl)methyl)phenoxy)piperidin-1-yl)-5-ethylpyrimidine A8 (50 mg, 0.093 mmol), 2-methoxyethylamine (9.6 μL, 0.11 mmol), Xantphos (5.4 mg, 0.0093 mmol), sodium tert-butoxide (26 mg, 0.28 mmol) and Pd₂(dba)₃ (4 mg, 0.0046 mmol) were dissolved in dioxane (1 mL) and degassed for 20 min with argon. Then the reaction vessel was sealed and heated at 120° C. for 12 h. The mixture was cooled, filtered, concentrated and purified by reverse-phase HPLC (H₂O/AcN gradient) to afford the title compound C9 as a white solid: ¹H NMR (400 MHz, CDCl₃) δ=8.11 (s, 2H), 6.69 (d, J=8.0 Hz, 1H), 6.53 (d, J=1.6 Hz, 1H), 6.49 (dd, J=2.0, 8.0 Hz, 1H), 4.45 (m, 1H), 4.10 (m, 2H), 3.55 (m, 4H), 3.41 (s, 2H), 3.29 (s, 3H), 3.22 (m, 6H), 2.71 (s, 3H), 2.51 (m, 4H), 2.39 (q, J=7.6 Hz, 2H), 1.97 (m, 4H), 1.76 (m, 2H), 1.12 (t, J=7.6 Hz, 3H); MS m/z for (M+H)⁺ C₂₆H₄₁N₆O₄S calc. 533.3, found 533.2.

### Example C10

### 4-(2-(1-(5-Ethylpyrimidin-2-yl)piperidin-4-yloxy)-5-((4-(methylsulfonyl)piperazin-1-yl)methyl)phenyl)morpholine

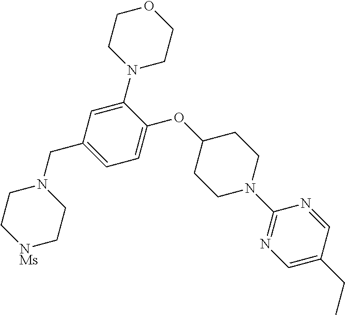

By following the same procedure as in C9 except using morpholine as the amine, the title compound C10 was obtained; ¹H NMR (400 MHz, CDCl₃) δ=8.12 (s, 2H), 6.82 (m, 3H), 4.55 (m, 1H), 4.03 (m, 2H), 3.77 (m, 4H), 3.65 (m, 2H), 3.22 (m, 4H), 3.03 (m, 4H), 2.73 (m, 4H), 2.40 (q, J=7.6 Hz, 2H), 1.95 (m, 3H), 1.78 (m, 3H), 1.12 (t, J=7.6 Hz, 3H); MS m/z for (M+H)⁺ C₂₇H₄₁N₆O₄S calc. 545.3, found 545.2.

### Example D1

### 3-(4-(3-Chloro-4-(1-(5-ethylpyrimidin-2-yl)piperidin-4-yloxy)benzyl)piperazin-1-ylsulfonyl)propan-1-ol

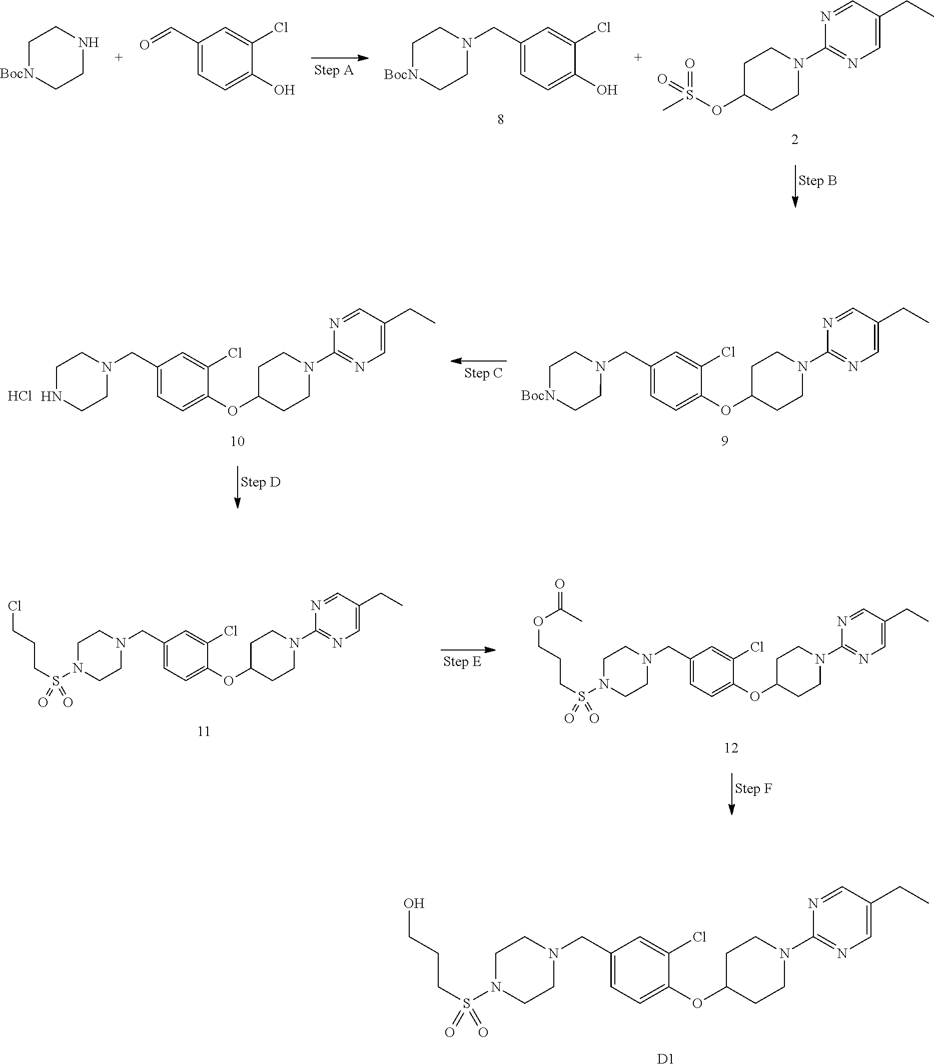

Step A: 3-Chloro-4-hydroxybenzaldehyde (500 mg, 3.19 mmol) and N-tert-butyl piperazine-1-carboxylate (595 mg, 3.19 mmol) were dissolved in dichloroethane (20 mL). AcOH (50 μL) was added, and the mixture was heated at 80° C. for 1 h. Sodium triacetoxyborohydride (1.35 g, 6.38 mmol) was added and the mixture was heated at 80° C. for 2 h. The reaction was cooled, diluted with sat. aq. NaHCO₃ (50 mL), and extracted with DCM (30 mL). The organic layer was washed with brine, dried (MgSO₄), filtered, concentrated and purified by flash column chromatography (SiO₂, MeOH/DCM gradient) to give N-tert-butyl 4-(3-chloro-4-hydroxybenzyl)piperazine-1-carboxylate 8 as a white powder: ¹H NMR (400 MHz, CDCl₃) δ=7.29 (d, J=2.0, 1H), 7.11 (dd, J=2.0, 8.0 Hz, 1H), 6.95 (d, J=8.0 Hz, 1H), 3.44 (m, 6H), 2.39 (m, 4H), 1.47 (s, 9H). MS m/z for (M+H)⁺ C₁₆H₂₄ClN₂O₃ calc. 327.1, found 327.2.

Step B: 4-(3-Chloro-4-hydroxybenzyl)piperazine-1-carboxylate 8 (940 mg, 2.87 mmol), 1-(5-ethylpyrimidin-2-yl)piperidin-4-yl methanesulfonate 2 (1.23 g, 4.31 mmol) and Cs₂CO₃ (1.8 g, 5.74 mmol) were heated in AcN (5 mL) at 60° C. for 12 h. The reaction was cooled, filtered, concentrated and purified by flash column chromatography (SiO₂, EtOAc/Hexane gradient) to give N-tert-butyl 4-(3-chloro-4-(1-(5-ethylpyrimidin-2-yl)piperidin-4-yloxy)benzyl)piperazine-1-carboxylate 9: MS m/z for (M+H)⁺ C₂₇H₃₉ClN₅O₃ calc. 516.3, found 516.3.

Step C: N-tert-Butyl 4-(3-chloro-4-(1-(5-ethylpyrimidin-2-yl)piperidin-4-yloxy)benzyl)piperazine-1-carboxylate 9 (345 mg, 0.67 mmol) was dissolved in TFA (4 mL) and stirred at rt for 1 h. The mixture was basified with sat. aq. NaHCO₃ (20 mL) and extracted with DCM (20 mL). The organic layer was dried (MgSO₄), filtered, and concentrated to give 2-(4-(2-chloro-4-(piperazin-1-ylmethyl)phenoxy)piperidin-1-yl)-5-ethylpyrimidine 10, which was used directly in Step D without further purification: MS m/z for (M+H)⁺ C₁₇H₂₈N₃O₃S calc. 416.2, found 416.2.

Step D: 2-(4-(2-Chloro-4-(piperazin-1-ylmethyl)phenoxy)piperidin-1-yl)-5-ethylpyrimidine 10 (163 mg, 0.39 mmol) was dissolved in DCM (5 mL). Triethylamine (60 μL, 0.43 mmol) was added and the mixture was cooled to 0° C. 3-Chloropropanesulfonyl chloride (52 μL, 0.43 mmol) was added and the mixture was stirred at rt for 1 h, then was diluted with H₂O (10 mL) extracted with DCM. The organic layer was dried (MgSO₄), filtered, and concentrated to give crude 2-(4-(2-chloro-4-((4-(3-chloropropylsulfonyl)piperazin-1-yl)methyl)phenoxy)-piperidin-1-yl)-5-ethylpyrimidine 11, which was used directly in Step E without further purification: MS m/z for (M+H)⁺ C₂₅H₃₆Cl₂N₅O₃S calc. 556.2, found 556.1.

Step E: 2-(4-(2-Chloro-4-((4-(3-chloropropylsulfonyl)piperazin-1-yl)methyl)phenoxy)piperidin-1-yl)-5-ethylpyrimidine 11 (0.39 mmol), NaI (60 mg, 0.395 mmol) and NaOAc (96 mg, 1.17 mmol) were dissolved in DMF (2 mL) and heated at 120° C. for 2 h. The mixture was cooled, diluted with H₂O (10 mL) and extracted with EtOAc (20 mL). The organic layer was washed with H₂O(10 mL) and brine (10 mL), then dried (MgSO₄), filtered and concentrated to provide 3-(4-(3-chloro-4-(1-(5-ethylpyrimidin-2-yl)piperidin-4-yloxy)benzyl)piperazin-1-ylsulfonyl)propyl acetate 12 which was used directly in Step F without further purification: MS m/z for (M+H)⁺ C₂₇H₃₉ClN₅O₅S calc. 580.2, found 580.1.

Step F: 3-(4-(3-Chloro-4-(1-(5-ethylpyrimidin-2-yl)piperidin-4-yloxy)benzyl)piperazin-1-ylsulfonyl)propyl acetate 12 (0.39 mmol) and LiOH—H₂O (50 mg, 1.19 mmol) were dissolved in THF (3 mL) and H₂O (25 μL) and heated at 60° C. for 2 h. The reaction was concentrated and purified by flash column chromatography (SiO2, EtOAc/Hexane gradient) to provide the title compound D1 as a white powder: ¹H NMR (400 MHz, CDCl₃) δ=8.11 (s, 2H), 7.28 (d, J=2.0, 1H), 7.05 (dd, J=2.0, 8.0 Hz, 1H), 6.87 (d, J=8.0 Hz, 1H), 4.51 (m, 1H), 4.02 (m, 2H), 3.69 (m, 2H), 3.62 (t, J=6.0 Hz, 2H), 3.39 (s, 2H), 3.25 (m, 4H), 3.01 (m, 2H), 2.46 (m, 4H), 2.39 (q, J=7.2 Hz, 2H), 2.23 (m, 2H), 1.92 (m, 2H), 1.82 (m, 2H), 1.12 (t, J=7.2 Hz, 3H). MS m/z for (M+H)⁺ C₂₅H₃₇ClN₅O₄S calc. 538.2, found 538.2.

### Example E1

### 5-Ethyl-2-(4-(4-((1-(methylsulfonyl)piperidin-4-yl)methyl)phenoxy)piperidin-1-yl)pyrimidine

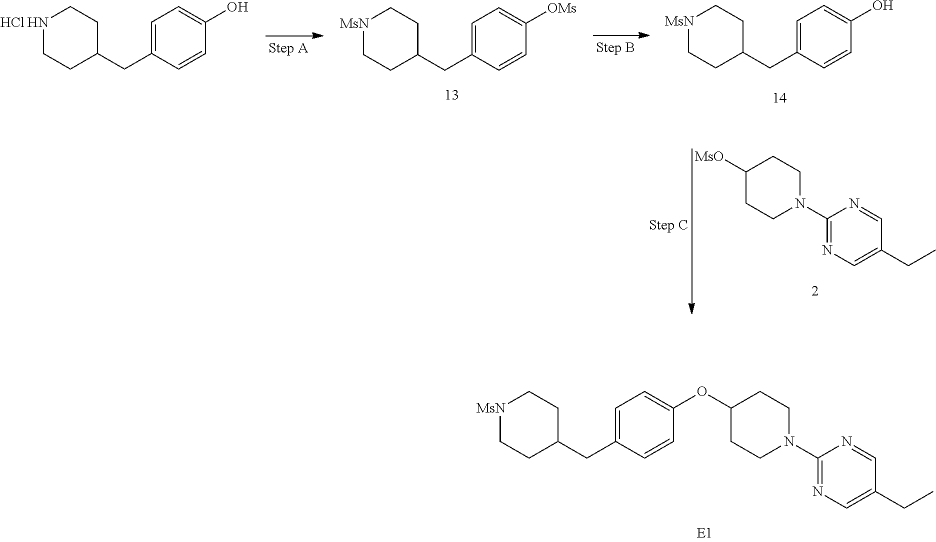

Step A: 4-(Piperidin-4-ylmethyl)phenol hydrochloride (500 mg, 2.19 mmol) was dissolved in DCM (20 mL), then diisopropylethylamine (1.15 mL, 6.58 mmol) and methanesulfonyl chloride (0.34 mL, 4.38 mmol) were added and stirred at rt for 2 h. The mixture was basified with sat. aq. NaHCO₃ (20 mL) and separated. The organic layer was washed with 1N HCl (10 mL) and brine (10 mL), dried (MgSO₄), filtered, and concentrated to provide 4-((1-(methylsulfonyl)piperidin-4-yl)methyl)phenyl methanesulfonate 13 which was used in Step B without further purification: MS m/z for (M+H)⁺ C₁₄H₂₂NO₅S₂ calc. 348.1, found 348.2.

Step B: 4-((1-(Methylsulfonyl)piperidin-4-yl)methyl)phenyl methanesulfonate 13 (2.19 mmol) was dissolved in MeOH (3 mL), then 10% NaOH (2 mL) was added and the mixture was heated at 80° C. for 1 h. The reaction was cooled, quenched with 1N HCl (10 mL) and extracted with EtOAc (20 mL). The organic layer was dried (MgSO₄), filtered, concentrated, and purified by flash column chromatography (SiO2, EtOAc/Hexane gradient) to provide 4-((1-(methylsulfonyl)piperidin-4-yl)methyl)phenol 14 as a white powder: ¹H NMR (400 MHz, CDCl₃) δ=6.92 (d, J=8.4 Hz, 2H), 6.69 (d, J=8.4 Hz, 2H), 3.70 (m, 2H), 2.68 (s, 3H), 2.51 (dt, J=2.4, 8.0 Hz, 2H), 2.43 (d, J=7.2 Hz, 2H), 1.67 (m, 2H), 1.49 (m, 1H), 1.26 (m, 2H). MS m/z for (M+H)⁺ C₁₃H₂₀NO₃S calc. 270.1, found 270.1.

Step C: 4-((1-(Methylsulfonyl)piperidin-4-yl)methyl)phenol 14 (50 mg, 0.19 mmol), 1-(5-ethylpyrimidin-2-yl)piperidin-4-yl methanesulfonate 2 (79 mg, 0.28 mmol) and Cs₂CO₃ (121 mg, 0.37 mmol) were heated in AcN (5 mL) at 60° C. for 12 h. The reaction was cooled, filtered, concentrated and purified by flash column chromatography (SiO2, EtOAc/Hexane gradient) to give the title compound E1 as a white powder: ¹H NMR (400 MHz, CDCl₃) δ=8.31 (s, 2H), 7.07 (d, J=8.8 Hz, 2H), 6.88 (d, J=8.8 Hz, 2H), 4.56 (m, 1H), 4.13 (m, 2H), 3.81 (m, 4H), 2.77 (s, 3H), 2.61 (m, 2H), 2.53 (m, 4H), 2.02 (m, 2H), 1.90 (m, 2H), 1.78 (m, 2H), 1.61 (m, 1H), 1.36 (m, 2H), 1.23 (t, J=7.6 Hz, 3H). MS m/z for (M+H)⁺ C₂₄H₃₅N₄O₃S calc. 459.2, found 459.2.

### Example E2

### 2-(4-(2-Chloro-4-((1-(methylsulfonyl)piperidin-4-yl)methyl)phenoxy)piperidin-1-yl)-5-ethylpyrimidine

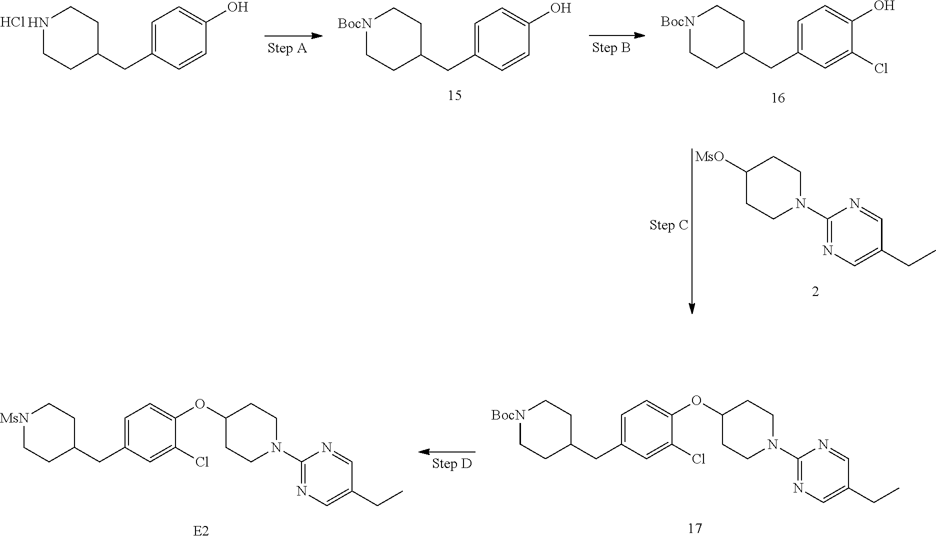

Step A: 4-(Piperidin-4-ylmethyl)phenol hydrochloride (380 mg, 1.67 mmol), Boc₂O (400 mg, 1.83 mmol), and sodium bicarbonate (1.4 g, 16.7 mmol) were dissolved in H₂O (10 mL), and dioxane (10 mL) and stirred at rt for 2 h. Then the mixture was extracted with EtOAc (20 mL), washed with brine (10 mL), dried (MgSO₄), filtered, and concentrated to provide 4-((1-(methylsulfonyl)piperidin-4-yl)methyl)phenyl methanesulfonate 15 which was used in Step B without further purification: MS m/z for (M−C₄H₉+H)⁺ C₁₃H₁₈NO₃ calc. 236.1, found 236.1.

Step B: N-tert-Butyl 4-(4-hydroxybenzyl)piperidine-1-carboxylate 15 (236 mg, 0.811 mmol) was dissolved in DCM (3 mL), then sulfuryl chloride (33 μL, 0.40 mmol) was added and the mixture was stirred at rt for 1 h. The reaction was diluted with water (10 mL) and extracted with DCM (20 mL). The organic layer was dried (MgSO₄), filtered, concentrated, and purified by flash column chromatography (SiO2, EtOAc/Hexane gradient) to provide N-tert-butyl 4-(3-chloro-4-hydroxybenzyl)piperidine-1-carboxylate 16 as a white powder. MS m/z for (M−C₄H₉+H)⁺ C₁₃H₁₇ClNO₃ calc. 270.1, found 270.1.

Step C: Following the same procedure as in D1 Step C except using N-tert-butyl 4-(3-chloro-4-hydroxybenzyl)piperidine-1-carboxylate 16 as the phenol, N-tert-butyl 4-(3-chloro-4-(1-(5-ethylpyrimidin-2-yl)piperidin-4-yloxy)benzyl)piperidine-1-carboxylate 17 was obtained. MS m/z for (M+H)⁺ C₂₈H₄₀ClN₄O₃ calc. 515.3, found 514.9.

Step D: N-tert-Butyl 4-(3-chloro-4-(1-(5-ethylpyrimidin-2-yl)piperidin-4-yloxy)benzyl)piperidine-1-carboxylate 17 (345 mg, 0.67 mmol) was dissolved in TFA (4 mL) and stirred at rt for 1 h. The mixture was basified with sat. aq. NaHCO₃ (20 mL) and extracted with DCM (20 mL). The organic layer was dried (MgSO₄), filtered, and concentrated. The remainder was dissolved in DCM (3 mL) and treated with diisopropylethylamine (34 μL, 0.19 mmol) and methanesulfonyl chloride (8 μL, 0.10 mmol). The mixture was stirred at rt for 1 h, then it was concentrated and purified by reverse-phase HPLC (H₂O/AcN gradient) to afford the title compound E2 as a white solid: ¹H NMR (400 MHz, CDCl₃) δ=8.20 (s, 2H), 7.18 (d, J=2.0 Hz, 1H), 6.98 (dd, J=2.0, 8.0 Hz, 1H), 6.93 (d, J=8.4 Hz, 1H), 4.57 (m, 1H), 4.13 (m, 2H), 3.77 (m, 4H), 2.78 (s, 3H), 2.62 (td, J=2.4, 12.0 Hz, 2H), 2.50 (m, 4H), 1.99 (m, 2H), 1.90 (m, 2H), 1.77 (m, 2H), 1.62 (m, 2H), 1.34 (m, 2H), 1.12 (t, J=7.6 Hz, 3H); MS m/z for (M+H)⁺ C₂₄H₃₄ClN₄O₃S calc. 493.2, found 493.2.

### Example E3

### 3-(4-(3-Chloro-4-(1-(5-ethylpyrimidin-2-yl)piperidin-4-yloxy)benzyl)piperidin-1-ylsulfonyl)propan-1-ol

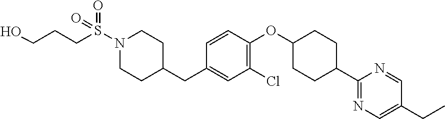

By following the same procedures as Dl except using N-tert-Butyl 4-(3-chloro-4-(1-(5-ethylpyrimidin-2-yl)piperidin-4-yloxy)benzyl)piperidine-1-carboxylate 17, the title compound E3 was obtained; ¹H NMR (400 MHz, CDCl₃) δ=8.02 (s, 2H), 6.99 (d, J=2.0 Hz, 1H), 6.79 (dd, J=2.0, 8.4 Hz, 1H), 6.75 (d, J=8.4 Hz, 1H), 4.39 (m, 1H), 3.94 (m, 2H), 3.59 (m, 6H), 2.87 (m, 2H), 2.55 (td, J=2.4, 12.0 Hz, 2H), 2.32 (m, 4H), 1.86 (m, 4H), 1.74 (m, 2H), 1.56 (m, 3H), 1.46 (m, 2H), 1.14 (m, 3H), 1.03 (t, J=7.6 Hz, 3H); MS m/z for (M+H)⁺ C₂₆H₃₈ClN₄O₄S calc. 537.2, found 537.1.

### Example E4

### 1-(3-Chloro-4-(1-(5-ethylpyrimidin-2-yl)piperidin-4-yloxy)benzyl)-4-(methylsulfonyl)piperazin-2-one

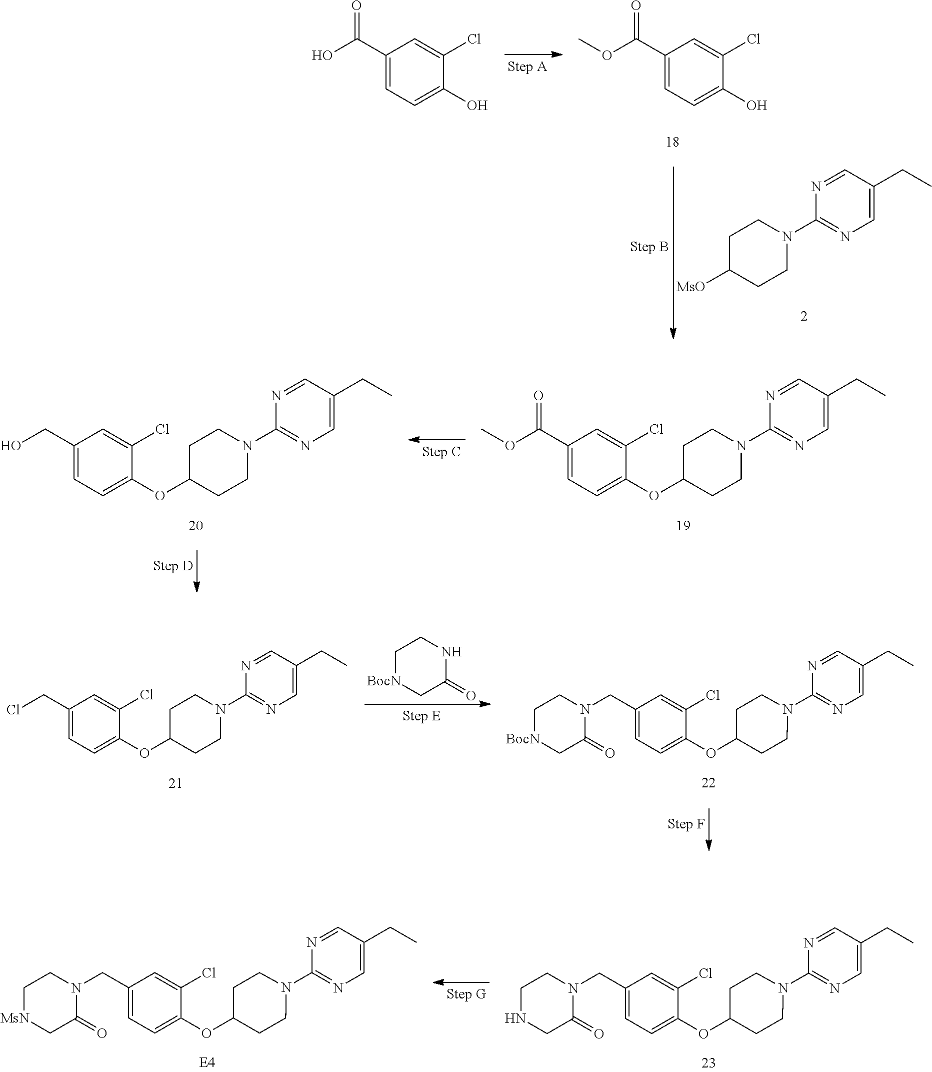

Step A: 3-Chloro-4-hydroxybenzoic acid (1 g, 5.79 mmol) was dissolved in MeOH (5 mL), then conc. H₂SO₄ (250 μL) was added and the mixture was heated at reflux for 3 h. The reaction was cooled, basified with sat. aq. NaHCO₃ (20 mL) and extracted with EtOAc (2×20 mL). The organic layers were combined, dried (MgSO₄), filtered and concentrated to give methyl 3-chloro-4-hydroxybenzoate 18: MS m/z for (M+H)⁺ C₈H₈ClO₃ calc. 187.0, found 187.1.

Step B: 1-(5-Ethylpyrimidin-2-yl)piperidin-4-yl methanesulfonate 2 (458 mg, 1.61 mmol), 3-chloro-4-hydroxybenzoate 18 (200 mg, 1.07 mmol) and Cs₂CO₃ (697 mg, 2.14 mmol) were dissolved in AcN (3 mL) and subjected to microwave irradiation (180° C., 3 min). The reaction was cooled, filtered, concentrated and purified by flash column chromatography (SiO2, EtOAc/Hexane gradient) to give methyl 3-chloro-4-(1-(5-ethylpyrimidin-2-yl)piperidin-4-yloxy)benzoate 19: ¹H NMR (400 MHz, CDCl₃) δ=8.21 (s, 2H), 8.09 (d, J=2.0 Hz, 1H), 7.93 (dd, J=2.0, 8.8 Hz, 1H), 7.01 (d, J=8.8 Hz, 1H), 4.76 (m, 1H), 4.06 (m, 2H), 3.92 (s, 3H), 3.87 (m, 2H), 2.48 (q, J=7.6 Hz, 2H), 2.04 (m, 2H), 1.94 (m, 2H), 1.22 (t, J=7.6 Hz, 3H). MS m/z for (M+H)⁺ C₁₉H₂₃ClN₃O₃ calc. 376.1, found 376.1.

Step C: Methyl 3-chloro-4-(1-(5-ethylpyrimidin-2-yl)piperidin-4-yloxy)benzoate 19 (536 mg, 1.53 mmol) was dissolved in dry THF (10 mL) and cooled to 0° C., then a 1 M solution of LiAlH₄ was added and the mixture was stirred at 0° C. for 10 min. The reaction was quenched by slow dropwise addition of H₂O, then was extracted with EtOAc (30 mL). The organic layer was washed with sat. aq. NaHCO₃ (10 mL), H₂O (10 mL), and brine (10 mL), then was dried (MgSO₄), filtered and concentrated to give crude (3-chloro-4-(1-(5-ethylpyrimidin-2-yl)piperidin-4-yloxy)phenyl)methanol 20, which was used in Step D without further purification. MS m/z for (M+H)⁺ C₁₈H₂₃ClN₃O₂ calc. 348.1, found 348.1.

Step D: (3-Chloro-4-(1-(5-ethylpyrimidin-2-yl)piperidin-4-yloxy)phenyl)methanol 20 (1.53 mmol) was dissolved in DCM (20 mL). Diisopropylethylamine (535 μL, 3.07 mmol) was added, followed by methanesulfonyl chloride (130 μL, 1.68 mmol) and the mixture was stirred at rt for 2 h. The mixture was concentrated and purified by flash column chromatography (SiO2, EtOAc/Hexane gradient) to give 2-(4-(2-chloro-4-(chloromethyl)phenoxy)piperidin-1-yl)-5-ethylpyrimidine 21: ¹H NMR (400 MHz, CDCl₃) δ=8.11 (s, 2H), 7.36 (d, J=2.4 Hz, 1H), 7.16 (dd, J=2.4, 8.4 Hz, 1H), 6.89 (d, J=8.4 Hz, 1H), 4.55 (m, 1H), 4.45 (m, 2H), 4.00 (m, 2H), 3.72 (m, 2H), 2.40 (q, J=7.6 Hz, 2H), 1.91 (m, 2H), 1.83 (m, 2H), 1.12 (t, J=7.6 Hz, 3H). MS m/z for (M+H)⁺ C₁₈H₂₂Cl₂N₃O calc. 366.1, found 366.1.

Step E: N-Boc-oxopiperazine (40 mg, 0.20 mmol) was dissolved in DMF (5 mL). Sodium hydride (12 mg, 0.3 mmol) was added and the mixture was heated at 60° C. for 1 h, then 2-(4-(2-Chloro-4-(chloromethyl)phenoxy)piperidin-1-yl)-5-ethylpyrimidine 21 (76 mg, 0.20 mmol) was added and the mixture was stirred at rt for 12 h. The mixture was quenched with H₂O (20 mL) and extracted with EtOAc (20 mL). The organic layer was washed with H₂O (20 mL) and brine (10 mL), then dried (MgSO₄), filtered, concentrated and purified by flash column chromatography (SiO2, EtOAc/Hexane gradient) to give N-tert-butyl 4-(3-chloro-4-(1-(5-ethylpyrimidin-2-yl)piperidin-4-yloxy)benzyl)-3-oxopiperazine-1-carboxylate 22: MS m/z for (M+H)⁺ C₂₇H₃₇ClN₅O₄ calc. 530.2, found 366.1.

Step F: N-tert-Butyl 4-(3-chloro-4-(1-(5-ethylpyrimidin-2-yl)piperidin-4-yloxy)benzyl)-3-oxopiperazine-1-carboxylate 22 (79 mg, 0.15 mmol) was dissolved in a mixture of DCM (2 mL) and a 4 N solution of HCl in dioxane (3 mL) and stirred at rt for 1 h. The mixture was basified with sat. aq. NaHCO₃ (40 mL) and extracted with DCM (20 mL). The organic layer was dried (MgSO₄), filtered, and concentrated to give 1-(3-chloro-4-(1-(5-ethylpyrimidin-2-yl)piperidin-4-yloxy)benzyl)piperazin-2-one 23, which was used directly in Step G without further purification: MS m/z for (M+H)⁺ C₂₂H₂₉ClN₅O₂ calc. 430.2, found 430.1.

Step G: 1-(3-Chloro-4-(1-(5-ethylpyrimidin-2-yl)piperidin-4-yloxy)benzyl)piperazin-2-one 23 (20 mg, 0.04 mmol) was dissolved in DCM (3 mL), then diisopropylethylamine (22 μL, 0.13 mmol) and methanesulfonyl chloride (4 μL, 0.05 mmol) were added and stirred at rt for 2 h. The mixture was basified with sat. aq. NaHCO₃ solution (10 mL), and extracted with DCM (10 mL). The organic layer was dried (MgSO₄), filtered, concentrated and purified by flash column chromatography (SiO2, EtOAc/Hexane gradient) to give the title compound E4: ¹H NMR (400 MHz, CDCl₃) δ=8.36 (s, 2H), 7.25 (d, J=2.4 Hz, 1H), 7.08 (dd, J=2.4, 8.4 Hz, 1H), 6.87 (d, J=8.4 Hz, 1H), 4.66 (m, 1H), 4.48 (s, 2H), 4.17 (m, 2H), 3.94 (s, 2H) 3.86 (m, 2H), 3.42 (m, 2H), 3.33 (m, 2H), 2.80 (s, 3H), 2.52 (q, J=7.6 Hz, 2H), 1.95 (m, 4H), 1.19 (t, J=7.6 Hz, 3H). MS m/z for (M+H)⁺ C₂₃H₃₁ClN₅O₄S calc. 508.2, found 508.1.

### Example E5

### 2-(4-(2-chloro-4-((4-(methylsulfonyl)piperidin-1-yl)methyl)phenoxy)piperidin-1-yl)-5-ethylpyrimidine

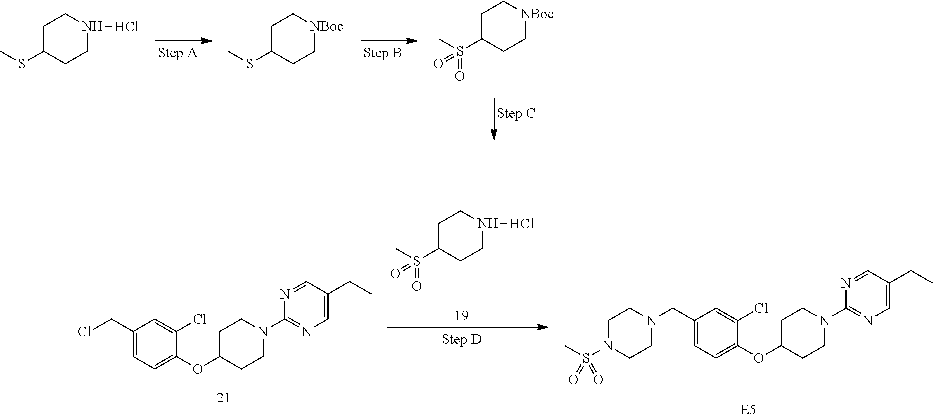

Step A: By following the same procedure as in in E2 Step A except using 4-methylthiopiperidine hydrochloride as the starting material, N-tert-butyl 4-(methylthio)piperidine-1-carboxylate was obtained.

Step B: N-tert-Butyl 4-(methylthio)piperidine-1-carboxylate (1.11 g, 4.8 mmol) and sodium periodate (5 g, 24 mmol) were dissolved in AcN (20 mL) and H₂O (8 mL) and heated at 100° C. for 2 h. The mixture was cooled, diluted with water (20 mL) and extracted with EtOAc (20 mL). The organic layer was washed with brine (20 mL), dried (MgSO₄), filtered, and concentrated to provide N-tert-butyl 4-(methylsulfonyl)piperidine-1-carboxylate: ¹H NMR (400 MHz, CDCl₃) δ=4.32 (s, 2H), 2.99 (tt, J=3.6, 12.4 Hz, 1H), 2.86 (s, 3H), 2.77 (m, 2H), 2.14 (d, J=14.0 Hz, 2H), 1.73 (m, 2H), 1.48 (s, 9H). MS m/z for (M−C₃H₉+H)⁺ C₇H₁₄NO₄S calc. 208.1, found 208.1.

Step C: N-tert-Butyl 4-(methylsulfonyl)piperidine-1-carboxylate (0.77 g, 2.91 mmol) was dissolved in HCl (10 mL of 4N in dioxane) and stirred at rt for 2 h. The reaction was concentrated to provide 4-(methylsulfonyl)piperidine hydrochloride 19, which was used directly in Step D without further purification. MS m/z for (M+H)⁺ C₆H₁₄NO₂S calc. 164.1, found 164.1.

Step D: 4-Methylthiopiperidine hydrochloride (19 mg, 0.09 mmol), 2-(4-(2-chloro-4-(chloromethyl)phenoxy)piperidin-1-yl)-5-ethylpyrimidine 21 (23 mg, 0.06 mmol) and Cs₂CO₃ were heated at 60° C. in AcN for 12 h. The mixture was cooled, filtered, concentrated and purified by reverse-phase HPLC (H₂O/AcN gradient) to afford the title compound E5 as a white solid: ¹H NMR (400 MHz, CDCl₃) δ=8.11 (s, 2H), 7.27 (d, J=2.0 Hz, 1H), 7.10 (dd, J=2.0, 8.4 Hz, 1H), 6.88 (d, J=8.4 Hz, 1H), 4.53 (m, 1H), 4.02 (m, 2H), 3.70 (m, 2H), 3.46 (m, 2H), 3.07 (m, 2H), 2.78 (m, 4H), 2.39 (q, J=7.6 Hz, 2H), 1.94 (s, 3H), 1.93 (m, 2H), 1.84 (m, 2H), 1.12 (t, J=7.6 Hz, 3H); MS m/z for (M+H)⁺ C₂₄H₃₄ClN₄O₃S calc. 493.2, found 493.2.

### Example E6

### 2-(4-(2-Chloro-4-((2-methyl-4-(methylsulfonyl)piperazin-1-yl)methyl)phenoxy)piperidin-1-yl)-5-ethylpyrimidine

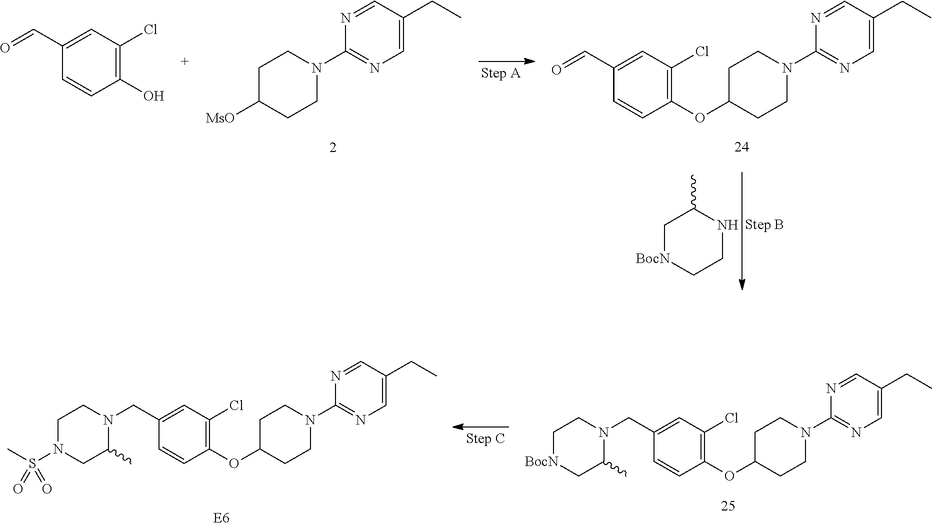

Step A: By following the same procedure as in A1 Step D except using 3-chloro-4-hydroxybenzaldehyde as the phenol, 3-chloro-4-(1-(5-ethylpyrimidin-2-yl)piperidin-4-yloxy)benzaldehyde 24 was obtained. MS m/z for (M+H)⁺ C₁₈H₂₁ClN₃O₂ calc. 346.1, found 346.1.

Step B: By following the same procedure as in A1 Step C except using (±)N-4-boc-2-methylpiperazine as the amine and 3-chloro-4-(1-(5-ethylpyrimidin-2-yl)piperidin-4-yloxy)benzaldehyde 24 as the aldehyde, N-tert-butyl 4-(3-chloro-4-(1-(5-ethylpyrimidin-2-yl)piperidin-4-yloxy)benzyl)-3-methylpiperazine-1-carboxylate 25 was obtained. MS m/z for (M+H)⁺ C₂₈H₄₁ClN₅O₃ calc. 530.3, found 530.0.

Step C: By following the same procedure as in E2 Step D the title compound E6 was obtained: ¹H NMR (400 MHz, CDCl₃) δ=8.20 (s, 2H), 7.38 (d, J=2.0 Hz, 1H), 7.15 (dd, J=2.0, 8.0 Hz, 1H), 6.96 (d, J=8.4 Hz, 1H), 4.61 (m, 1H), 4.11 (m, 2H), 3.79 (m, 2H), 3.46 (m, 2H), 2.79 (s, 3H), 2.48 (q, J=7.6 Hz, 2H), 1.99 (m, 2H), 1.91 (m, 2H), 1.24 (m, 6H), 0.09 (m, 1H); MS m/z for (M+H)⁺ C₂₄H₃₅ClN₅O₃S calc. 508.2, found 508.1.

### Example E7

### 2-(4-(2-Chloro-4-((2-methyl-4-(methylsulfonyl)piperazin-1-yl)methyl)phenoxy)piperidin-1-yl)-5-ethylpyrimidine

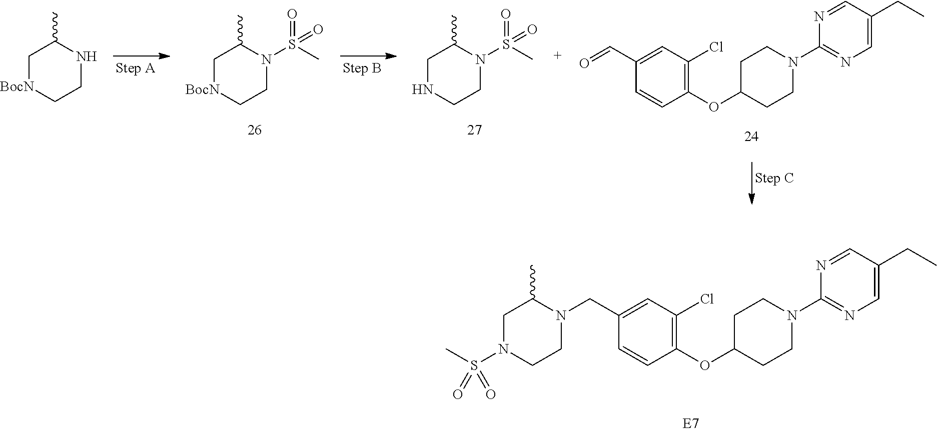

Step A: By following the same procedure as in E4 Step G except using (±)N-4-boc-2-methylpiperazine as the starting material, compound 26 was obtained. MS m/z for (M−C₃H₉+H)⁺ C₇H₁₅N₂O₄S calc. 223.1, found 223.1.

Step B: By following the same procedure as in E4 Step F except using 26 as the starting material, compound 27 was obtained. MS m/z for (M+H)^{±}C₆H₁₅N₂O₂S calc. 179.1, found 179.1.

Step C: By following the same procedure as in E6 Step C except using 27 as the starting material, the title compound E7 was obtained: ¹H NMR (400 MHz, CDCl₃) δ=8.11 (s, 2H), 7.28 (d, J=2.0 Hz, 1H), 7.07 (dd, J=2.0, 8.4 Hz, 1H), 6.87 (d, J=8.4 Hz, 1H), 4.52 (m, 1H), 4.02 (m, 3H), 3.70 (m, 2H), 3.37 (m, 4H), 2.79 (s, 3H), 2.39 (q, J=7.6 Hz, 2H), 1.92 (m, 2H), 1.93 (m, 2H), 1.31 (s, 1.5H), 1.30 (s. 1.5H), 1.12 (t, J=7.6 Hz, 3H), 0.80 (m, 1H); MS m/z for (M+H)⁺ C₂₄H₃₅ClN₅O₃S calc. 508.2, found 507.9.

### Example E8

### N-(3-Chloro-4-(1-(5-ethylpyrimidin-2-yl)piperidin-4-yloxy)phenyl)-N-methyl-1-(methylsulfonyl)piperidin-4-amine

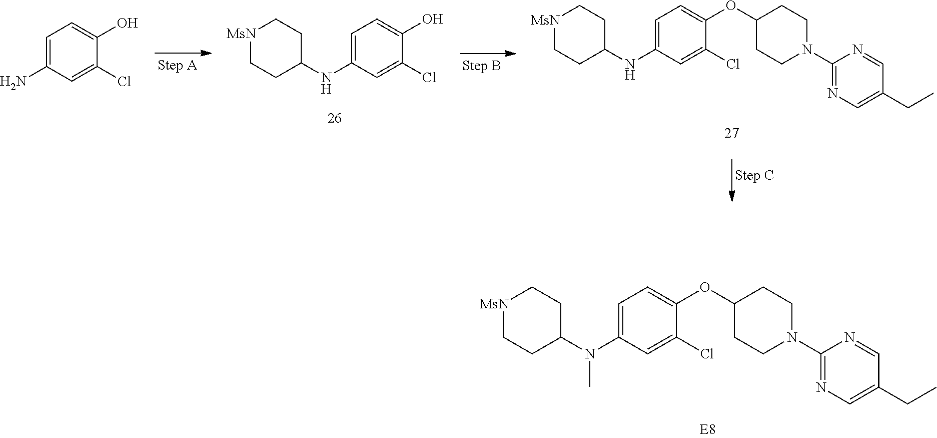

Step A: 4-Amino-2-chlorophenol (200 mg, 1.39 mmol) and 1-methanesulfonyl-4-piperidinone (247 mg, 1.39 mmol) were dissolved in 5% AcOH/EtOH (5 mL) and heated at 60° C. for 1 h. The mixture was cooled, then NaBH₃CN (175 mg, 2.78 mmol) was added and the mixture was stirred for 2 h at rt. The reaction mixture was diluted with saturated NaHCO₃ (10 mL) and extracted with EtOAc (20 mL). The organic layer was dried (MgSO₄), filtered and concentrated to provide 2-chloro-4-(1-(methylsulfonyl)piperidin-4-ylamino)phenol 26 which was used in the next step without further purification. MS m/z for (M+H)⁺ C₁₂H₁₈ClN₂O₃S calc. 305.1, found 305.1.

Step B: By following the same procedure as in A1 Step D except using 2-chloro-4-(1-(methylsulfonyl)piperidin-4-ylamino)phenol 26 as the phenol, N-(3-chloro-4-(1-(5-ethylpyrimidin-2-yl)piperidin-4-yloxy)phenyl)-1-(methylsulfonyl)piperidin-4-amine 27 was obtained: ¹H NMR (400 MHz, CDCl₃) δ=8.43 (s, 2H), 7.25 (d, J=2.8 Hz, 1H), 7.11 (dd, J=2.8, 8.8 Hz, 1H), 6.98 (d, J=8.8 Hz, 1H), 4.72 (m, 1H), 4.22 (m, 2H), 3.95 (m, 2H), 3.82 (m, 2H), 3.40 (m, 1H), 2.83 (m, 5H), 2.63 (q, J=7.6 Hz, 2H), 2.05 (m, 6H), 1.77 (m, 2H), 1.29 (t, J=7.6 Hz, 3H); MS m/z for (M+H)⁺ C₂₃H₃₃ClN₅O₃S calc.

Step C: N-(3-Chloro-4-(1-(5-ethylpyrimidin-2-yl)piperidin-4-yloxy)phenyl)-1-(methylsulfonyl)piperidin-4-amine 27 (20 mg, 0.0405 mmol) and paraformaldehyde (50 mg) were dissolved in 5% AcOH/EtOH (5 mL) and heated to 80° C. for 1 h. The mixture was cooled, then NaBH₃CN (60 mg, 0.95 mmol) was added and the mixture was stirred for 2 h at rt. The mixture was diluted with saturated NaHCO₃ (10 mL) and extracted with EtOAc (20 mL). The organic layer was washed with brine (10 mL), dried (MgSO₄), filtered, concentrated and purified by reverse-phase HPLC (H₂O/AcN gradient) to afford the title compound E8 as a white solid: ¹H NMR (400 MHz, CDCl₃) δ=8.44 (s, 2H), 7.19 (d, J=2.8 Hz, 1H), 7.10 (dd, J=2.8, 8.8 Hz, 1H), 7.00 (d, J=8.8 Hz, 1H), 4.66 (m, 1H), 4.18 (m, 2H), 3.98 (m, 4H), 3.61 (m, 1H), 2.98 (s, 3H), 2.84 (s, 3H), 2.80 (td, J=2.0, 12.0 Hz, 2H), 2.60 (q, J=7.6 Hz, 2H), 2.05 (m, 6H), 1.83 (m, 2H), 1.27 (t, J=7.6 Hz, 3H); MS m/z for M+H)⁺ C₂₄H₃₅ClN₅O₃S calc. 508.2, found 508.1.

### Example E9

### 4-(3-Chloro-4-(1-(5-ethylpyrimidin-2-yl)piperidin-4-yloxy)benzyl)piperazine-1-carbonitrile

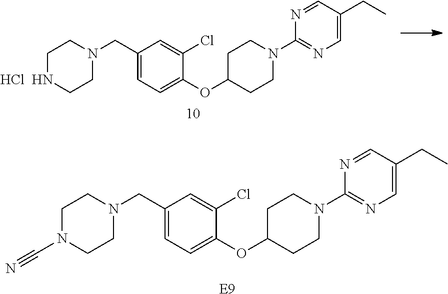

2-(4-(2-Chloro-4-(piperazin-1-ylmethyl)phenoxy)piperidin-1-yl)-5-ethylpyrimidine hydrochloride 10 (100 mg, 0.22 mmol), cyanogen bromide (26 mg, 0.24 mmol) and K₂CO₃ (66 mg, 0.48 mmol) were dissolved in a 1:1 mixture of H₂O and DCM (8 mL) and stirred at rt for 12 h. The mixture was extracted with DCM (5 mL) and the organic layer was dried (MgSO₄), filtered, concentrated and purified by reverse-phase HPLC (H₂O/AcN gradient) to provide the title compound E9 as a white solid. MS m/z for (M+H)⁺ C₂₃H₃₀ClN₆O calc. 441.2, found 441.1.

### Example E10

### 2-(4-(2-Chloro-4-((4-(2-methyl-2H-tetrazol-5-yl)piperazin-1-yl)methyl)phenoxy)piperidin-1-yl)-5-ethylpyrimidine

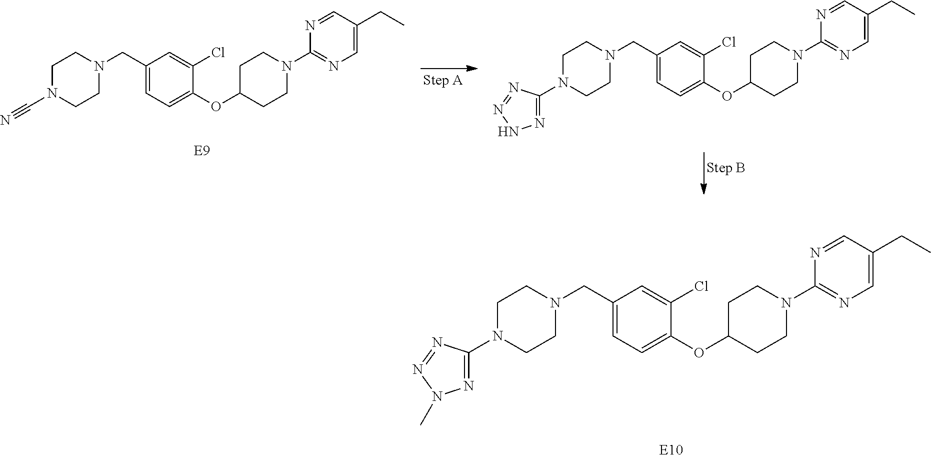

Step A: 4-(3-Chloro-4-(1-(5-ethylpyrimidin-2-yl)piperidin-4-yloxy)benzyl)piperazine-1-carbonitrile E9 (97 mg, 0.22 mmol), sodium azide (38 mg, 0.59 mmol) and NH₄Cl (39 mg, 0.73 mmol) were dissolved in DMF (2 mL) and heated at 90° C. for 12 h. The mixture was cooled, diluted with H₂O (10 mL) and extracted with EtOAc (20 mL). The organic layer was washed with H₂O (10 mL) and brine (10 mL), dried (MgSO₄), filtered, concentrated and purified by reverse-phase HPLC (H₂O/AcN gradient) to provide 2-(4-(4-((4-(2H-tetrazol-5-yl)piperazin-1-yl)methyl)-2-chlorophenoxy)piperidin-1-yl)-5-ethylpyrimidine as a white solid: MS m/z for (M+H)⁺ C₂₃H₃₁ClN₉O calc. 484.2, found 484.1.

Step B: 2-(4-(4-((4-(2H-Tetrazol-5-yl)piperazin-1-yl)methyl)-2-chlorophenoxy)piperidin-1-yl)-5-ethylpyrimidine (23 mg, 0.047 mmol), K₂CO₃ (50 mg), and iodomethane (3.5 μL, 0.056 mmol) were dissolved in acetone (2 mL) and stirred at rt for 3 h. The mixture was filtered, concentrated, and purified by reverse-phase HPLC (H₂O/AcN gradient) to provide the title compound E10 as a white solid: ¹H NMR (400 MHz, CDCl₃) δ=8.45 (s, 2H), 7.43 (d, J=2.0 Hz, 1H), 7.37 (dd, J=2.4, 8.4 Hz, 1H), 7.03 (d, J=8.4 Hz, 1H), 4.81 (m, 1H), 4.26 (m, 2H), 4.20 (m, 4H), 3.93 (m, 3H), 2.61 (q, J=7.6 Hz, 2H), 2.07 (m, 4H), 1.28 (t, J=7.6 Hz, 3H); MS m/z for (M+H)⁺ C₂₄H₃₃ClN₉O calc. 498.2, found 498.1.

### Example E11

### 1-(3-Chloro-4-(1-(5-ethylpyrimidin-2-yl)piperidin-4-yloxy)benzyl)piperidine-4-carbonitrile

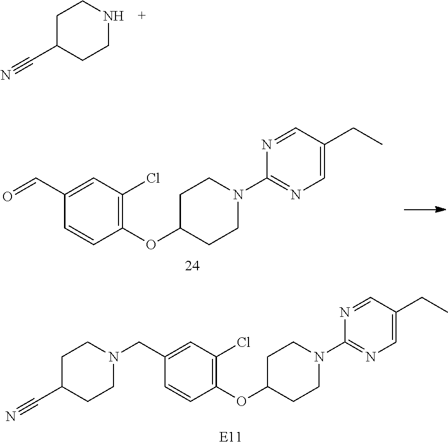

4-Cyanopiperidine (100 mg, 0.90 mmol), and 3-chloro-4-(1-(5-ethylpyrimidin-2-yl)piperidin-4-yloxy)benzaldehyde 24 (210 mg, 0.61 mmol) were dissolved in 5% AcOH/EtOH (5 mL) and heated to 60° C. for 1 h. The mixture was cooled, then NaBH₃CN (60 mg, 0.95 mmol) was added and the mixture was stirred for 2 h at rt. The mixture was diluted with saturated NaHCO₃ (10 mL) and extracted with EtOAc (20 mL). The organic layer was washed with brine (10 mL), dried (MgSO₄), filtered, concentrated and purified by flash column chromatography (SiO₂, MeOH/DCM gradient) to afford the title compound E11 as a white solid: ¹H NMR (400 MHz, CDCl₃) δ=8.11 (s, 2H), 7.26 (d, J=2.0 Hz, 1H), 7.04 (dd, J=2.0, 8.4 Hz, 1H), 6.86 (d, J=8.4 Hz, 1H), 4.50 (m, 1H), 4.03 (m, 2H), 3.69 (m, 2H), 3.34 (s, 2H), 2.57 (m, 3H), 2.39 (q, J=7.6 Hz, 2H), 2.24 (m, 2H), 1.87 (m, 8H), 1.12 (t, J=7.6 Hz, 3H); MS m/z for (M+H)⁺ C₂₄H₃₁ClN₅O calc. 440.2, found 440.1.

### Example E12

### 2-(4-(2-chloro-4-((4-(2-methyl-2H-tetrazol-5-yl)piperidin-1-yl)methyl)phenoxy)piperidin-1-yl)-5-ethylpyrimidine

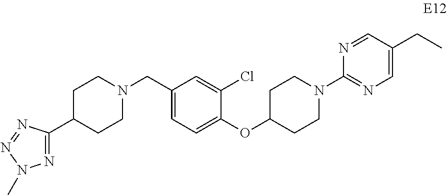

By following the same procedures as E10 except using 1-(3-Chloro-4-(1-(5-ethylpyrimidin-2-yl)piperidin-4-yloxy)benzyl)piperidine-4-carbonitrile E11 as the nitrile, the title compound E12 was obtained: ¹H NMR (400 MHz, CDCl₃) δ=8.11 (s, 2H), 7.30 (d, J=2.4 Hz, 1H), 7.09 (dd, J=2.0, 8.4 Hz, 1H), 6.87 (d, J=8.4 Hz, 1H), 4.51 (m, 1H), 4.23 (s, 3H), 4.02 (m, 2H), 3.68 (m, 2H), 3.38 (s, 2H), 2.86 (m, 3H), 2.39 (q, J=7.6 Hz, 2H), 1.85 (m, 10H), 1.12 (t, J=7.6 Hz, 3H); MS m/z for (M+H)⁺ C₂₅H₃₄ClN₈O calc. 497.2, found 497.2.

### Example E13

### 2-(4-(2-Chloro-4-((1-(methylsulfonyl)azetidin-3-yloxy)methyl)phenoxy)piperidin-1-yl)-5-ethylpyrimidine

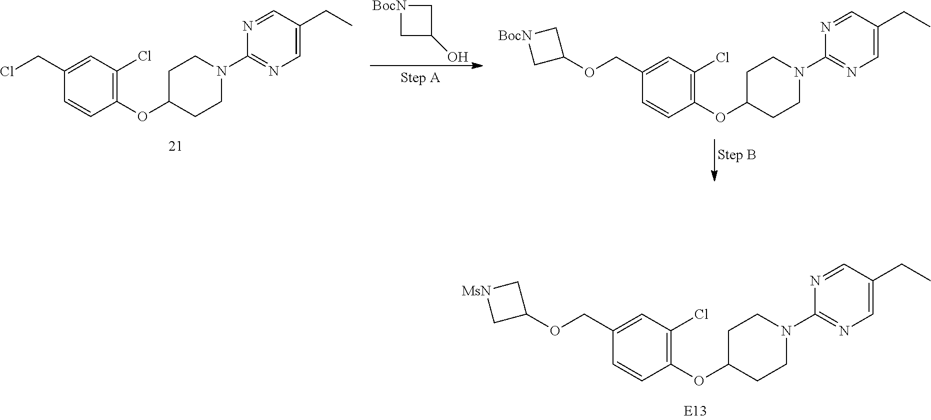

Step A: 2-(4-(2-Chloro-4-(chloromethyl)phenoxy)piperidin-1-yl)-5-ethylpyrimidine 21 (66 mg, 0.18 mmol), 1-boc-3-hydroxyazetidine (38 mg, 0.22 mmol) and Cs₂CO₃ (118 mg, 0.36 mmol) were dissolved in AcN (5 mL) and heated at 60° C. for 2 h. The mixture was cooled, filtered and concentrated to provide N-tert-butyl 3-(3-chloro-4-(1-(5-ethylpyrimidin-2-yl)piperidin-4-yloxy)benzyloxy)azetidine-1-carboxylate, which was used without further purification in the next step. MS m/z for (M+H)⁺ C₂₆H₃₆ClN₄O₄ calc. 503.2, found 503.1.

Step B: By following the same procedure as in E2 Step D the title compound E13 was obtained: ¹H NMR (400 MHz, CDCl₃) δ=8.21 (s, 2H), 7.28 (d, J=2.0 Hz, 1H), 7.09 (dd, J=2.0, 8.4 Hz, 1H), 6.90 (d, J=8.8 Hz, 1H), 4.59 (m, 1H), 4.31 (s, 2H), 4.26 (m, 1H), 3.97 (m, 4H), 3.90 (m, 2H), 3.78 (m, 2H), 2.81 (s, 3H), 2.44 (q, J=7.6 Hz, 2H), 1.92 (m, 5H), 1.24 (t, J=7.6 Hz, 3H); MS m/z for (M+H)⁺ C₂₂H₃₀ClN₄O₄S calc. 481.2, found 481.1.

### Example E14

### 2-(4-(2-Chloro-4-((1-(methylsulfonyl)azetidin-3-yloxy)methyl)phenoxy)piperidin-1-yl)-5-ethylpyrimidine

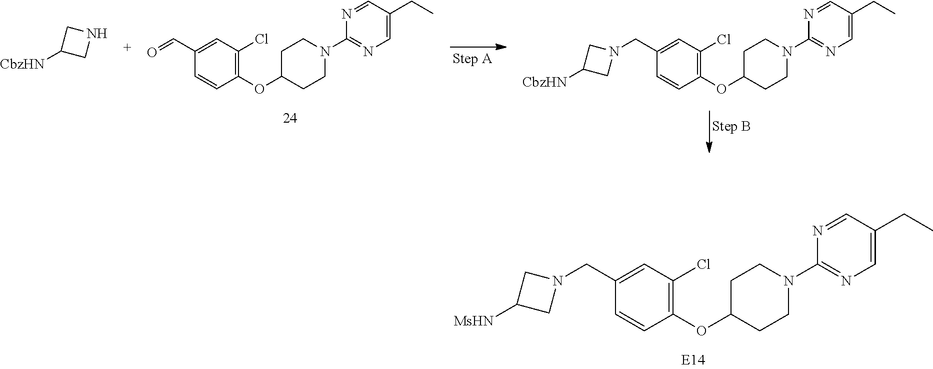

Step A: By following the same procedure as in A1 Step C except using benzyl azetidin-3-ylcarbamate as the amine and 3-chloro-4-(1-(5-ethylpyrimidin-2-yl)piperidin-4-yloxy)benzaldehyde 24 as the aldehyde, benzyl 1-(3-chloro-4-(1-(5-ethylpyrimidin-2-yl)piperidin-4-yloxy)benzyl)azetidin-3-ylcarbamate was obtained. MS m/z for (M+H)⁺ C₂₉H₃₅ClN₅O₃ calc. 536.3, found 536.3.

Step B: Benzyl 1-(3-chloro-4-(1-(5-ethylpyrimidin-2-yl)piperidin-4-yloxy)benzyl)azetidin-3-ylcarbamate (82 mg, 0.15 mmol) was dissolved in a 1:1 mixture of EtOH and EtOAc (5 mL). Pd/C (10 mg of 10%, wet) was added and the mixture was stirred under H₂ (1 atm) for 12 h. The mixture was filtered through a 0.2 μm syringe filter and concentrated. The residue was dissolved in THF (2 mL), NEt₃ (42 μL, 0.30 mmol) was added and this mixture was added dropwise to a solution of methanesulfonyl chloride (13 μL, 0.16 mmol) in THF (2 mL) and stirred at rt for 2 h. The mixture was concentrated and purified by reverse-phase HPLC (H₂O/AcN gradient) to provide the title compound E14 as a white powder: ¹H NMR (400 MHz, CDCl₃) δ=8.20 (s, 2H), 7.30 (d, J=2.4 Hz, 1H), 7.11 (dd, J=2.4, 8.4 Hz, 1H), 6.96 (d, J=8.4 Hz, 1H), 4.60 (m, 1H), 4.11 (s, 3H), 3.79 (m, 2H), 3.71 (m, 2H), 3.56 (s, 2H), 3.05 (m, 2H), 2.96 (s, 3H), 2.48 (q, J=7.6 Hz, 2H), 2.01 (m, 3H), 1.91 (m, 2H), 1.21 (t, J=7.6 Hz, 3H); MS m/z for (M+H)^{±}C₂₂H₃₁ClN₅O₃S calc. 480.2, found 480.1.

### Example E15

### N-(1-(3-Chloro-4-(1-(5-ethylpyrimidin-2-yl)piperidin-4-yloxy)benzyl)azetidin-3-yl)-N-methylmethanesulfonamide

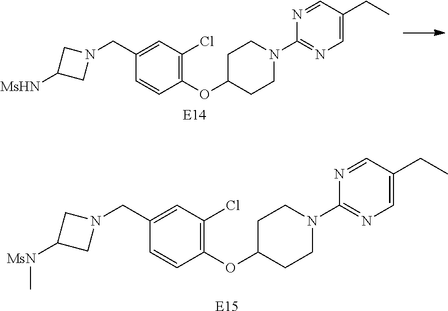

2-(4-(2-Chloro-4-((1-(methylsulfonyl)azetidin-3-yloxy)methyl)phenoxy)piperidin-1-yl)-5-ethylpyrimidine (18 mg, 0.038 mmol) was dissolved in THF (2 mL). NaH (2 mg, 0.046 mmol) was added and the mixture was heated at 60° C. for 1 h. The mixture was cooled, then iodomethane (4.7 μL, 0.076 mmol) was added and the mixture was stirred at 60° C. for 12 h. The reaction was quenched with H₂O (5 mL), and the mixture was extracted with EtOAc (10 mL). The organic layer was dried (MgSO₄), filtered, concentrated and purified by reverse-phase HPLC (H₂O/AcN gradient) to provide the title compound E15 as a white solid: ¹H NMR (400 MHz, CDCl₃) δ=8.11 (s, 2H), 7.23 (d, J=2.0 Hz, 1H), 7.02 (dd, J=2.4, 8.4 Hz, 1H), 6.86 (d, J=8.4 Hz, 1H), 4.51 (m, 1H), 4.14 (m, 1H), 4.03 (m, 2H), 3.69 (m, 2H), 3.48 (m, 3H), 3.09 (m, 2H), 2.80 (s, 3H), 2.68 (s, 3H), 2.39 (q, J=7.6 Hz, 2H), 1.90 (m, 3H), 1.83 (m, 2H), 1.12 (t, J=7.6 Hz, 3H); MS m/z for (M+H)⁺ C₂₃H₃₃ClN₅O₃S calc. 494.2, found 494.1.

### Example F1

### N-tert-Butyl 4-(2-fluoro-4-((4-(methylsulfonyl)piperazin-1-yl)methyl)phenylamino)-piperidine-1-carboxylate

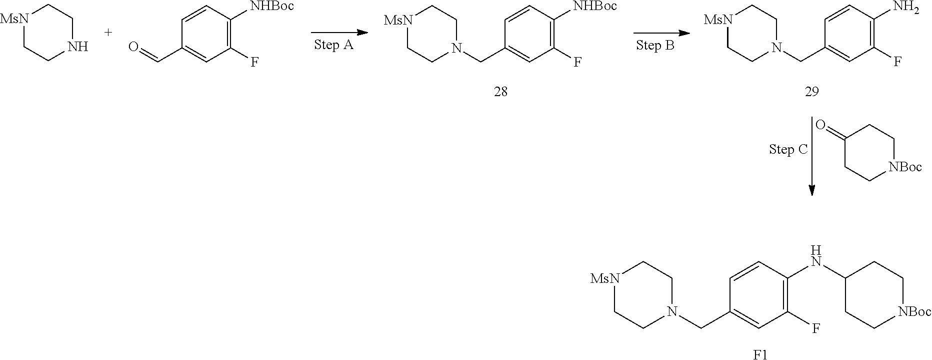

Step A: N-tert-Butyl 2-fluoro-4-formylphenylcarbamate (767 mg, 3.2 mmol) and 1-methanesulfonylpiperazine (580 mg, 3.53 mmol) were dissolved in 5% AcOH/95% EtOH (20 mL) and the mixture was heated at 60° C. for 1 h. The reaction was cooled, then NaBH₃CN (603 mg, 9.60 mmol) was added and the mixture was stirred at rt for 2 h. The reaction was cooled, diluted with sat. aq. NaHCO₃ (50 mL), and extracted with EtOAc (50 mL). The organic layer was washed with brine, dried (MgSO₄), filtered, concentrated and purified by flash column chromatography (SiO₂, EtOAc/Hexane gradient) to give N-tert-butyl 2-fluoro-4-((4-(methylsulfonyl)piperazin-1-yl)methyl)phenylcarbamate 28 as a white powder: ¹H NMR (400 MHz, CDCl₃) δ=8.03 (m, 1H), 7.06 (m, 2H), 6.70 (s, 1H), 3.49 (s, 2H), 3.26 (m, 4H), 2.80 (s, 3H), 2.55 (m, 4H), 1.55 (s, 9H). MS m/z for (M+H)⁺ C₁₇H₂₇FN₃O₄S calc. 388.2, found 388.2.

Step B: N-tert-Butyl 2-fluoro-4-((4-(methylsulfonyl)piperazin-1-yl)methyl)phenylcarbamate 28 (742 mg, 1.91 mmol) was dissolved in a mixture of DCM (5 mL) and a 4 N solution of HCl in dioxane (5 mL) and stirred at rt for 1 h. The mixture was basified with sat. aq. NaHCO₃ (40 mL) and extracted with DCM (20 mL). The organic layer was dried (MgSO₄), filtered, and concentrated to give 2-fluoro-4-((4-(methylsulfonyl)-piperazin-1-yl)methyl)aniline 29 (481 mg, 88%), which was used directly in Step B without further purification: MS m/z for (M+H)⁺ C₁₂H₁₉FN₃O₂S calc. 288.1, found 288.1.

Step C: 2-Fluoro-4-((4-(methylsulfonyl)piperazin-1-yl)methyl)aniline 29 (481 mg, 1.67 mmol) and 4-oxo-1-Boc-piperidine (367 mg, 1.84 mmol) were dissolved in 5% AcOH/EtOH (10 mL) and the mixture was heated at 60° C. for 1 h. The reaction was cooled, then NaBH₃CN (314 mg, 5.01 mmol) was added and the mixture was stirred at rt for 2 h. The reaction was cooled, diluted with sat. aq. NaHCO₃ (50 mL), and extracted with EtOAc (50 mL). The organic layer was washed with brine, dried (MgSO₄), filtered, concentrated and purified by flash column chromatography (SiO₂, MeOH/DCM gradient) to give the title compound E1 as a white powder: ¹H NMR (400 MHz, CDCl₃) δ=7.03 (m, 2H), 6.71 (t, J=8.4 Hz, 1H), 4.07 (m, 4H), 3.48 (m, 2H), 2.97 (m, 2H), 2.88 (s, 3H), 2.05 (m, 2H), 1.49 (s, 9H), 1.42 (m, 2H). MS m/z for (M+Na)⁺ C₂₂H₃₆FN₄O₄SNa calc. 493.2, found 493.2, found 493.1.

### Biological Assays

Generation of Stable Cell Line

Flp-In-CHO cells (Invitrogen, Cat.#R758-07) are maintained in Ham's F12 medium supplemented with 10% fetal bovine serum, 1% antibiotic mixture and 2 mM L-glutamine. The cells are transfected with a DNA mixture containing human GPR119 in pcDNA5/FRT vector and the pOG44 vector (1:9) using Fugene6 (Roche), according to the manufacturer's instruction. After 48 hours, the medium is changed to medium supplemented with 400 μg/ml hygromycin B to initiate the selection of stably transfected cells.

Cyclic AMP Assay in Stable Cell Line

To test the activity of compounds of the invention, Flp-In-CHO-hGPR119 cells are harvested and resuspended in DMEM plus 3% lipid-depleted fetal bovine serum. Forth μl of cells are plated in 384 well plates at a density of 15,000 cells/well. IBMX (3-isobutyl-1-methyl-xanthine) is added to the cells to a final concentration of 1 mM, followed by the addition of 500 nl of the compound to be tested. The cells are incubated at 37° C. for 30 minutes. Equal volume (20 μl) of the HTRF reagents, anti-cAMP-Cryptate and cAMP-XL665, are added to the cells. The plates are incubated at room temperature for 1 hour and read on a HTRF reader according to the manufacturer's instruction.

Compounds of Formula I, in free form or in pharmaceutically acceptable salt form, produced a concentration-dependent increase in intracellular cAMP level. Compound of the invention show an EC₅₀ of between 1×10⁻⁵ and 1×10⁻¹⁰M, preferably less than 500 nM, more preferably less than 100 nM. For example, the following tables show some EC₅₀ data for a representative sample of compounds of the invention:

Table of Biological Activity

| Example | CHO-GPR119-HTRF (3158) μM |
|---|---|
| A1 | 0.056 |
| A2 | 0.203 |
| A6 | 0.020 |
| A8 | 0.005 |
| A13 | 0.867 |
| A14 | 0.313 |
| B2 | 0.127 |
| B3 | 0.022 |
| B6 | 0.220 |
| B11 | 0.013 |
| B13 | 0.465 |
| B17 | 0.009 |
| C2 | 0.187 |
| C5 | 0.044 |
| C9 | 0.136 |
| D1 | 0.008 |
| E2 | 0.003 |
| E4 | 0.812 |
| E12 | 2.68 |
| E15 | 0.114 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 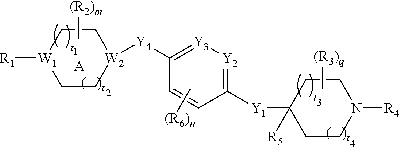
in which:
A can have up to 2 ring —CH₂— group substituted with —C(O)— and can be partially unsaturated with up to 2 double bonds;
m and n are independently selected from 0, 1, 2, 3 and 4;
q is selected from 0, 1, 2, 3 and 4;
t₁ t₂, t₃ and t₄ are each independently selected from 0, 1 and 2;
R₁ is selected from hydrogen, cyano, —X₁S(O)₀₋₂X₂R₆ₐ, —X₁N(S(O)₀₋₂X₂R₆ₐ)R₆ₐ, —X₁S(O)₀₋₂X₂OR₆ₐ, —X₁S(O)₀₋₂X₂C(O)R₆ₐ, —X₁C(O)OR₆ₐ, —X₁R₆ₐ, —X₁S(O)₀₋₂X₂C(O)OR₆ₐ and —X₁S(O)₀₋₂NR₆ₐR_{6b}; wherein X₁ is selected from a bond, O, —NR₇ₐR_{7b} and C₁₋₄ alkylene; X₂ is selected from a bond and C₁₋₄alkylene; R₆ₐ is selected from hydrogen, C₁₋₆alkyl, C₆₋₁₀ aryl, C₁₋₁₀heteroaryl, C₃₋₈heterocycloalkyl and C₁₋₈cycloalkyl; wherein said aryl, heteroaryl, cycloalkyl and heterocycloalkyl of R₆ₐ is optionally substituted with 1 to 3 radicals independently selected from hydroxy, halo, C₁₋₆alkyl, halo-substituted-C₁₋₆alkyl, hydroxy-substituted-C₁₋₆alkyl, C₁₋₆ alkoxy, halo-substituted-C₁₋₆ alkoxy and C₆₋₁₀ aryl-C₁₋₄alkoxy; R_{6b} is selected from hydrogen and C₁₋₆alkyl; and R₇ₐ and R_{7b} are independently selected from hydrogen and C₁₋₆alkyl;
R₂ and R₃ are independently selected from halo, hydroxy, C₁₋₆alkyl, halo-substituted-C₁₋₆ alkyl, hydroxy-substituted-C₁₋₆alkyl, C₁₋₆ alkoxy, halo-substituted-C₁₋₆ alkoxy, —C(O)R₈, and —C(O)OR₈; wherein R₈ is selected from hydrogen and C₁₋₆alkyl;
R₄ is selected from R₉ and —C(O)OR₉; wherein R₉ is selected from C₁₋₆alkyl, C₆₋₁₀aryl, C₁₋₁₀heteroaryl, C₃₋₈cycloalkyl and C₃₋₈heterocycloalkyl; wherein said aryl, heteroaryl, cycloalkyl or heterocycloalkyl of R₉ is optionally substituted with 1 to 3 radicals independently selected from halo, cyano, C₁₋₆alkyl, C₃₋₁₂cycloalkyl, C₃₋₈heterocycloalkyl, halo-substituted-C₁₋₆alkyl, hydroxy-substituted-C₁₋₆alkyl, C₁₋₆alkoxy, halo-substituted-C₁₋₆alkoxy and —C(O)OR₁₇, —C(O)R₁₉ and —C(O)NR₁₇R₁₈; wherein R₁₇ and R₁₈ are independently selected from hydrogen and C₁₋₆alkyl; or R₁₇ and R₁₈ together with the nitrogen atom to which R₁₇ and R₁₈ are attached form C₃₋₈heterocycloalkyl; R₁₉ is selected from C₁₋₆alkyl and C₃₋₈heterocycloalkyl; wherein said cycloalkyl or heterocycloalkyl substituents of R₉ are optionally further substituted with 1 to 3 C₁₋₆alkyl radicals;
R₅ is selected from hydrogen, C₁₋₆alkyl, halo-substituted-C₁₋₆alkyl, hydroxy-substituted-C₁₋₆alkyl, C₁₋₆alkoxy and halo-substituted-C₁₋₆alkoxy;
R₆ is selected from hydroxy, nitro, cyano, halo, C₁₋₆alkyl, C₂₋₆alkenyl, halo-substituted-C₁₋₆alkyl, halo-substituted-C₂₋₆alkenyl, hydroxy-substituted-C₁₋₆alkyl, C₁₋₆alkoxy, halo-substituted-C₁₋₆alkoxy, C₆₋₁₀aryl, C₁₋₁₀heteroaryl, C₃₋₈heterocycloalkyl, C₃₋₈cycloalkyl and —X₃OR₂₀, —NR₂₀X₃OR₂₁, —C(O)OR₂₀; wherein X₃ is selected from a bond, C₁₋₄alkylene and C₂₋₄alkenylene; R₂₀ and R₂₁ are independently selected from hydrogen and C₁₋₆alkyl;
W₁ and W₂ are independently selected from CR₁₀ and N; wherein R₁₀ is selected from hydrogen and C₁₋₆alkyl;
Y₁ is selected from NR₁₁, O and S; wherein R₁₁ is selected from hydrogen and C₁₋₆alkyl;
Y₂ and Y₃ are independently selected from CH and N;
Y₄ is selected from CH₂, OCH₂ and NR₁₅; wherein R₁₅ is selected from hydrogen and C₁₋₆alkyl; or the pharmaceutically acceptable salts thereof.

2. The compound of claim 1 of Formula Ia: 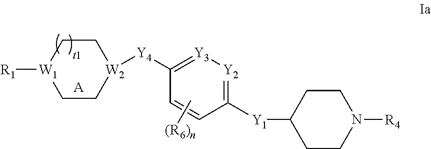
in which:
A can have a ring —CH₂— group substituted with —C(O)—;
t1 is selected from 0 and 1;
R₁ is selected from hydrogen, cyano, —X₁S(O)₀₋₂X₂R₆ₐ, —X₁S(O)₀₋₂X₂OR₆ₐ, —X₁C(O)OR₆ₐ, —X₁S(O)₀₋₂X₂C(O)R₆ₐ, —X₁N(S(O)₀₋₂X₂R₆ₐ)R₆ₐ, —X₁S(O)₀₋₂X₂C(O)OR₆ₐ and —X₁S(O)₀₋₂NR₆ₐR_{6b}; wherein X₁ is selected from a bond, O, —NR₇ₐR_{7b} and C₁₋₄alkylene; X₂ is selected from a bond and C₁₋₄alkylene; R₆ₐ is selected from hydrogen, C₁₋₆alkyl, C₆₋₁₀ aryl, C₁₋₁₀heteroaryl, C₃₋₈heterocycloalkyl and C₁₋₈ cycloalkyl; wherein said aryl, heteroaryl, cycloalkyl and heterocycloalkyl of R₆ₐ is optionally substituted with 1 to 3 radicals independently selected from hydroxy, halo, C₁₋₆alkyl, halo-substituted-C₁₋₆alkyl, hydroxy-substituted-C₁₋₆alkyl, C₁₋₆ alkoxy, halo-substituted-C₁₋₆ alkoxy and C₆₋₁₀ aryl-C₁₋₄alkoxy; R_{6b} is selected from hydrogen and C₁₋₆alkyl; and R₇ₐ and R_{7b} are independently selected from hydrogen and C₁₋₆alkyl;
R₄ is selected from R₉ and —C(O)OR₉; wherein R₉ is selected from C₁₋₆alkyl, C₆₋₁₀aryl, C₁₋₁₀heteroaryl, C₃₋₈cycloalkyl and C₃₋₈heterocycloalkyl; wherein said aryl, heteroaryl, cycloalkyl or heterocycloalkyl of R₉ is optionally substituted with 1 to 3 radicals independently selected from halo, cyano, C₁₋₆alkyl, C₃₋₁₂cycloalkyl, C₃₋₈heterocycloalkyl, halo-substituted-C₁₋₆alkyl, hydroxy-substituted-C₁₋₆alkyl, C₁₋₆alkoxy, halo-substituted-C₁₋₆alkoxy and —C(O)OR₁₇, —C(O)R₁₉ and —C(O)NR₁₇R₁₈; wherein R₁₇ and R₁₈ are independently selected from hydrogen and C₁₋₆alkyl; or R₁₇ and R₁₈ together with the nitrogen atom to which R₁₇ and R₁₈ are attached form C₃₋₈heterocycloalkyl; R₁₉ is selected from C₁₋₆alkyl and C₃₋₈heterocycloalkyl; wherein said cycloalkyl or heterocycloalkyl substituents of R₉ are optionally further substituted with 1 to 3 C₁₋₆alkyl radicals;
R₆ is selected from hydroxy, nitro, cyano, halo, C₁₋₆alkyl, C₂₋₆alkenyl, halo-substituted-C₁₋₆alkyl, halo-substituted-C₂₋₆alkenyl, hydroxy-substituted-C₁₋₆alkyl, C₁₋₆alkoxy, halo-substituted-C₁₋₆alkoxy, C₆₋₁₀ aryl, C₁₋₁₀heteroaryl, C₃₋₈heterocycloalkyl, C₃₋₈cycloalkyl and —X₃OR₂₀, —NR₂₀X₃OR₂₁, —C(O)OR₂₀; wherein X₃ is selected from a bond, C₁₋₄alkylene and C₂₋₄alkenylene; R₂₀ and R₂₁ are independently selected from hydrogen and C₁₋₆alkyl;
W₂ is selected from CR₁₀ and N; wherein R₁₀ is selected from hydrogen and C₁₋₆alkyl;
Y₁ is selected from NH, O and S; and
Y₂ and Y₃ are independently selected from CH and N;
Y₄ is selected from CH₂, OCH₂ and NR₁₅; wherein R₁₅ is selected from hydrogen and C₁₋₆alkyl.

3. The compound of claim 2 in which: A can have a ring —CH₂— group substituted with —C(O)—; t1 is selected from 0 and 1; and R₁ is selected from hydrogen, cyano, —S(O)₀₋₂X₂R₆ₐ, —X₁N(S(O)₀₋₂X₂R₆ₐ)R₆ₐ, —X₁R₆ₐ, —X₁C(O)OR₆ₐ and —S(O)₀₋₂X₂OR₆ₐ; wherein X₁ is selected from a bond and C₁₋₄alkylene; X₂ is selected from a bond and C₁₋₄alkylene; R₆ₐ is selected from hydrogen, C₁₋₆alkyl and C₁₋₁₀heteroaryl optionally substituted with C₁₋₆alkyl.

4. The compound of claim 3 in which: R₄ is selected from R₉ and —C(O)OR₉; wherein R₉ is selected from tert-butyl, pyridinyl, pyrimidinyl, 1,2,4-oxadiazol-5-yl, tetrazolyl and cyclopropyl; wherein said pyridinyl, pyrimidinyl, 1,2,4-oxadiazol-5-yl, tetrazolyl or cyclopropyl of R₉ is optionally substituted with a radical selected from halo, cyano, trifluoromethyl, isopropyl, methyl, ethyl, methoxy-carbonyl, dimethyl-amino-carbonyl, amino-carbonyl and morpholino-carbonyl.

5. The compound of claim 4 in which R₆ is selected from fluoro, chloro, bromo, trifluoromethoxy, methyl, methoxy, methoxy-carbonyl, 3-methoxyprop-1-enyl, methoxy-propyl, vinyl, phenyl, pyrazolyl, 5-chloropent-1-enyl, hydroxy-propyl, methoxy-ethyl-amino and morpholino; W₂ is selected from CH and N; Y₁ is selected from NH, O and S; and Y₂ and Y₃ are independently selected from CH and N; Y₄ is selected from CH₂, OCH₂ and NCH₃.

6. The compound of claim 5 selected from: 5-Ethyl-2-(4-(4-((4-(methylsulfonyl)piperazin-1-yl)methyl)phenoxy)piperidin-1-yl)pyrimidine; 5-ethyl-2-(4-(3-methyl-4-((4-(methylsulfonyl)piperazin-1-yl)methyl)phenoxy)piperidin-1-yl)pyrimidine; 5-ethyl-2-(4-(3-methoxy-4-((4-(methylsulfonyl)piperazin-1-yl)methyl)phenoxy)piperidin-1-yl)pyrimidine; 5-ethyl-2-(4-(3-fluoro-4-((4-(methylsulfonyl)piperazin-1-yl)methyl)phenoxy)piperidin-1-yl)pyrimidine; 2-(4-(3-chloro-4-((4-(methylsulfonyl)piperazin-1-yl)methyl)phenoxy)piperidin-1-yl)-5-ethylpyrimidine; 5-ethyl-2-(4-(2-methyl-4-((4-(methylsulfonyl)piperazin-1-yl)methyl)phenoxy)piperidin-1-yl)pyrimidine; 5-ethyl-2-(4-(2-fluoro-4-((4-(methylsulfonyl)piperazin-1-yl)methyl)phenoxy)piperidin-1-yl)pyrimidine; 2-(4-(2-chloro-4-((4-(methylsulfonyl)piperazin-1-yl)methyl)phenoxy)piperidin-1-yl)-5-ethylpyrimidine; 2-(4-(2-bromo-4-((4-(methylsulfonyl)piperazin-1-yl)methyl)phenoxy)piperidin-1-yl)-5-ethylpyrimidine; 5-ethyl-2-(4-(2-methoxy-4-((4-(methylsulfonyl)piperazin-1-yl)methyl)phenoxy)piperidin-1-yl)pyrimidine; 5-ethyl-2-(4-(4-((4-(methylsulfonyl)piperazin-1-yl)methyl)-2-(trifluoromethoxy)phenoxy)piperidin-1-yl)pyrimidine; 2-(4-(2,3-difluoro-4-((4-(methylsulfonyl)piperazin-1-yl)methyl)phenoxy)piperidin-1-yl)-5-ethylpyrimidine; 2-(4-(2-chloro-6-fluoro-4-((4-(methylsulfonyl)piperazin-1-yl)methyl)phenoxy)piperidin-1-yl)-5-ethylpyrimidine; 5-ethyl-2-(4-(6-((4-(methylsulfonyl)piperazin-1-yl)methyl)pyridin-3-yloxy)piperidin-1-yl)pyrimidine; 5-ethyl-2-(4-(5-((4-(methylsulfonyl)piperazin-1-yl)methyl)pyridin-2-yloxy)piperidin-1-yl)pyrimidine; tert-Butyl 4-(4-((4-(methylsulfonyl)piperazin-1-yl)methyl)phenoxy)piperidine-1-carboxylate; 1-methylcyclopropyl 4-(4-((4-(methylsulfonyl)piperazin-1-yl)methyl)phenoxy)piperidine-1-carboxylate; 5-Fluoro-2-(4-(4-((4-(methylsulfonyl)piperazin-1-yl)methyl)phenoxy)piperidin-1-yl)pyrimidine; 1-(4-(1-(5-fluoropyridin-2-yl)piperidin-4-yloxy)benzyl)-4-(methylsulfonyl)piperazine; 1-(methylsulfonyl)-4-(4-(1-(5-(trifluoromethyl)pyridin-2-yl)piperidin-4-yloxy)benzyl)piperazine; 5-(4-(2-chloro-4-((4-(methylsulfonyl)piperazin-1-yl)methyl)phenoxy)piperidin-1-yl)-3-isopropyl-1,2,4-oxadiazole; 3-(4-(3-Chloro-4-(1-(5-ethylpyrimidin-2-yl)piperidin-4-yloxy)benzyl)piperazin-1-ylsulfonyl)propan-1-ol; 5-Ethyl-2-(4-(4-((1-(methylsulfonyl)piperidin-4-yl)methyl)phenoxy)piperidin-1-yl)pyrimidine; tert-Butyl 4-(2-fluoro-4-((4-(methylsulfonyl)piperazin-1-yl)methyl)phenylamino) piperidine-1-carboxylate; 1-(3-Chloro-4-(1-(5-ethylpyrimidin-2-yl)piperidin-4-yloxy)benzyl)-4-(methylsulfonyl)piperazin-2-one, methyl 2-(1-(5-ethylpyrimidin-2-yl)piperidin-4-yloxy)-5-((4-(methylsulfonyl)piperazin-1-yl)methyl)benzoate; 1-methylcyclopropyl 4-(2-chloro-4-((4-(methylsulfonyl)piperazin-1-yl)methyl)phenoxy)piperidine-1-carboxylate; methyl 2-(4-(2-chloro-4-((4-(methylsulfonyl)piperazin-1-yl)methyl)phenoxy)piperidin-1-yl)pyrimidine-5-carboxylate; 2-(4-(2-chloro-4-((4-(methylsulfonyl)piperazin-1-yl)methyl)phenoxy)piperidin-1-yl)pyrimidine; 5-bromo-2-(4-(2-chloro-4-((4-(methylsulfonyl)piperazin-1-yl)methyl)-phenoxy)piperidin-1-yl)pyrimidine; 5-chloro-2-(4-(2-chloro-4-((4-(methylsulfonyl)piperazin-1-yl)methyl)phenoxy)piperidin-1-yl)pyrimidine; 2-(4-(2-Chloro-4-((4-(methylsulfonyl)-piperazin-1-yl)methyl)phenoxy)piperidin-1-yl)pyrimidine-5-carboxamide; 2-(4-(2-chloro-4-((4-(methylsulfonyl)piperazin-1-yl)methyl)phenoxy)piperidin-1-yl)-N,N-dimethylpyrimidine-5-carboxamide; (2-(4-(2-chloro-4-((4-(methylsulfonyl)piperazin-1-yl)methyl)phenoxy)piperidin-1-yl)pyrimidin-5-yl)(morpholino)methanone; 2-(4-(2-Chloro-4-((4-(methylsulfonyl)piperazin-1-yl)methyl)phenoxy)piperidin-1-yl)pyrimidine-5-carbonitrile; 2-(4-(2-Chloro-4-((4-(methylsulfonyl)piperazin-1-yl)methyl)phenoxy)piperidin-1-yl)-5-(2H-tetrazol-5-yl)pyrimidine; 2-(4-(2-Chloro-4-((4-(methylsulfonyl)piperazin-1-yl)methyl)phenoxy)piperidin-1-yl)-5-(2-methyl-2H-tetrazol-5-yl)pyrimidine; (E)-5-Ethyl-2-(4-(2-(3-methoxyprop-1-enyl)-4-((4-(methylsulfonyl)piperazin-1-yl)methyl)phenoxy)piperidin-1-yl)pyrimidine; 3-(2-(1-(5-Ethylpyrimidin-2-yl)piperidin-4-yloxy)-5-((4-(methylsulfonyl)piperazin-1-yl)methyl)phenyl)propan-1-ol; 5-ethyl-2-(4-(4-((4-(methylsulfonyl)piperazin-1-yl)methyl)-2-vinylphenoxy)piperidin-1-yl)pyrimidine; 5-ethyl-2-(4-(5-((4-(methylsulfonyl)piperazin-1-yl)methyl)biphenyl-2-yloxy)piperidin-1-yl)pyrimidine; 5-ethyl-2-(4-(4-((4-(methylsulfonyl)piperazin-1-yl)methyl)-2-(1H-pyrazol-5-yl)phenoxy)piperidin-1-yl)pyrimidine; (E)-2-(4-(2-(5-chloropent-1-enyl)-4-((4-(methylsulfonyl)piperazin-1-yl)methyl)phenoxy)piperidin-1-yl)-5-ethylpyrimidine; (E)-4-(2-(1-(5-Ethylpyrimidin-2-yl)piperidin-4-yloxy)-5-((4-(methylsulfonyl)piperazin-1-yl)methyl)phenyl)but-3-en-1-ol; 3-(2-(1-(5-Ethylpyrimidin-2-yl)piperidin-4-yloxy)-5-((4-(methylsulfonyl)piperazin-1-yl)methyl)phenyl)propan-1-ol; 2-(1-(5-Ethylpyrimidin-2-yl)piperidin-4-yloxy)-N-(2-methoxyethyl)-5-((4-(methylsulfonyl)piperazin-1-yl)methyl)aniline; 4-(2-(1-(5-Ethylpyrimidin-2-yl)piperidin-4-yloxy)-5-((4-(methylsulfonyl)piperazin-1-yl)methyl)phenyl)morpholino; 2-(4-(2-Chloro-4-((1-(methylsulfonyl)piperidin-4-yl)methyl)phenoxy)piperidin-1-yl)-5-ethylpyrimidine; 3-(4-(3-Chloro-4-(1-(5-ethylpyrimidin-2-yl)piperidin-4-yloxy)benzyl)piperidin-1-ylsulfonyl)propan-1-ol; 2-(4-(2-chloro-4-((4-(methylsulfonyl)piperidin-1-yl)methyl)phenoxy)piperidin-1-yl)-5-ethylpyrimidine; 2-(4-(2-Chloro-4-((2-methyl-4-(methylsulfonyl)piperazin-1-yl)methyl)phenoxy)piperidin-1-yl)-5-ethylpyrimidine; 2-(4-(2-Chloro-4-((2-methyl-4-(methylsulfonyl)piperazin-1-yl)methyl)phenoxy)piperidin-1-yl)-5-ethylpyrimidine; N-(3-Chloro-4-(1-(5-ethylpyrimidin-2-yl)piperidin-4-yloxy)phenyl)-N-methyl-1-(methylsulfonyl)piperidin-4-amine; 4-(3-Chloro-4-(1-(5-ethylpyrimidin-2-yl)piperidin-4-yloxy)benzyl)piperazine-1-carbonitrile; 2-(4-(2-Chloro-4-((4-(2-methyl-2H-tetrazol-5-yl)piperazin-1-yl)methyl)phenoxy)piperidin-1-yl)-5-ethylpyrimidine; 1-(3-Chloro-4-(1-(5-ethylpyrimidin-2-yl)piperidin-4-yloxy)benzyl)piperidine-4-carbonitrile; 2-(4-(2-chloro-4-((4-(2-methyl-2H-tetrazol-5-yl)piperidin-1-yl)methyl)phenoxy)piperidin-1-yl)-5-ethylpyrimidine; 2-(4-(2-Chloro-4-((1-(methylsulfonypazetidin-3-yloxy)methyl)phenoxy)piperidin-1-yl)-5-ethylpyrimidine; 2-(4-(2-Chloro-4-((1-(methylsulfonyl)azetidin-3-yloxy)methyl)phenoxy)piperidin-1-yl)-5-ethylpyrimidine; and N-(1-(3-Chloro-4-(1-(5-ethylpyrimidin-2-yl)piperidin-4-yloxy)benzyl)azetidin-3-yl)-N-methylmethanesulfonamide.

7. A pharmaceutical composition comprising a therapeutically effective amount of a compound of claim 1 in combination with a pharmaceutically acceptable excipient.

8. A method for modulating GPR119 activity, comprising administering to a system or a subject in need thereof, a therapeutically effective amount of the compound of claim 1 or pharmaceutically acceptable salts or pharmaceutical compositions thereof, thereby modulating said GPR119 activity.

9. The method of claim 8, wherein the compound of claim 1 directly contacts GPR119.

10. The method of claim 11, wherein the contacting occurs in vitro or in vivo.

11. A method for treating a disease or condition wherein modulation of GPR119 activity can prevent, inhibit or ameliorate the pathology and/or symptomology of the disease or condition, comprising administering to a subject a therapeutically effective amount of the compound of claim 1 or pharmaceutically acceptable salts or pharmaceutical compositions thereof.

12. The method of claim 11, wherein said disease or condition is selected from obesity, type 1 diabetes, type 2 diabetes mellitus, hyperlipidemia, idiopathic type 1 diabetes, latent autoimmune diabetes in adults, early-onset type 2 diabetes, youth-onset atypical diabetes, maturity onset diabetes of the young, malnutrition-related diabetes and gestational diabetes.

13. The method of claim 11, wherein said disease or condition is selected from coronary heart disease, ischemic stroke, restenosis after angioplasty, peripheral vascular disease, intermittent claudication, myocardial infarction, dyslipidemia, post-prandial lipemia, conditions of impaired glucose tolerance, conditions of impaired fasting plasma glucose, metabolic acidosis, ketosis, arthritis, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.
